(19) United States
(12) Patent Application Publication
(10) Pub. No.: US 2025/0084161 A1
(43) Pub. Date: Mar. 13, 2025

(54) ANTI P2X7 RECEPTOR ANTIBODIES AND FRAGMENTS THEREOF

(71) Applicant: BIOSCEPTRE (AUST) PTY LTD, North Ryde (AU)

(72) Inventors: Julian Alexander Barden, North Ryde (AU); Neil Brewis, Stevenage (GB); Philip Jones, Cambridge (GB); Steven Grant, Cambridge (GB)

(21) Appl. No.: 18/895,282

(22) Filed: Sep. 24, 2024

Related U.S. Application Data
(63) Continuation of application No. 17/198,179, filed on Mar. 10, 2021, now Pat. No. 12,129,295.
Application No. 17/198,179 is a continuation of application No. 16/032,973, filed on Jul. 11, 2018, now Pat. No. 10,988,532.
Application No. 16/032,973 is a continuation of application No. 15/599,396, filed on May 18, 2017, now Pat. No. 10,053,508.
Application No. 15/599,396 is a continuation of application No. 14/671,254, filed on Mar. 27, 2015, now Pat. No. 9,688,771.
Application No. 14/671,254 is a continuation of application No. 13/391,619, filed on Feb. 21, 2012, now Pat. No. 9,127,059.

(30) Foreign Application Priority Data
Aug. 20, 2009 (AU) 2009903928

(51) Int. Cl. C07K 16/28; C07K 16/30

(52) U.S. Cl. CPC C07K 16/28; C07K 16/30; C07K 2317/34; C07K 2317/565; C07K 2317/567; C07K 2317/569; C07K 2317/74; C07K 2317/76; C07K 2317/92

(57) ABSTRACT

The invention relates to an antigen binding site for binding to a P2X₇ receptor, the antigen binding site being defined by general formula 1: FR1-CDR1-FR2-CDR2-FR3-CDR3-FR4

### CROSS-REFERENCE TO RELATED APPLICATIONS

The present application is a continuation of U.S. application Ser. No. 16/032,973 filed Jul. 11, 2018, which is a continuation of U.S. application Ser. No. 15/599,396 filed May 18, 2017 now U.S. Pat. No. 10,053,508, which is a continuation of U.S. application Ser. No. 14/671,254 filed Mar. 27, 2015 now U.S. Pat. No. 9,688,771, which is a continuation of U.S. application Ser. No. 13/391,619 filed Feb. 21, 2012 now U.S. Pat. No. 9,688,771, which is the national stage of PCT/AU2010/001070 filed Aug. 20, 2010, each incorporated by reference in its entirety for all purposes, which claims priority to Australian Application No. 2009903928 filed Aug. 20, 2009.

### REFERENCE TO SEQUENCE LISTING

This application includes an electronic sequence listing in a file named 554704SEQLST.TXT, created on Mar. 10, 2021 and containing 104,241 bytes, which is incorporated by reference.

### FIELD OF THE INVENTION

The invention relates to purinergic receptors, to antibodies and related fragments thereof for binding to said receptors, to production of said antibodies and fragments and to use of said antibodies and fragments for cancer detection and therapy.

### BACKGROUND OF THE INVENTION

Reference to any prior art in the specification is not, and should not be taken as, an acknowledgment or any form of suggestion that this prior art forms part of the common general knowledge in Australia or any other jurisdiction or that this prior art could reasonably be expected to be ascertained, understood and regarded as relevant by a person skilled in the art.

Purinergic (P2X) receptors are ATP-gated cation-selective channels. Each receptor is made up of three protein subunits or monomers. To date seven separate genes encoding P2X monomers have been identified: P2X1, P2X2, P2X3, P2X4, P2X5, P2X6, P2X₇.

P2X₇ receptors are of particular interest as the expression of these receptors is understood to be limited to cells having potential to undergo programmed cell death, such as thymocytes, dendritic cells, lymphocytes, macrophages and monocytes. There is some expression of P2X₇ receptors in normal homeostasis, such as on erythrocytes.

Interestingly, a P2X₇ receptor containing one or more monomers having a cis isomerisation at Pro210 (according to SEQ ID NO: 1) and which is devoid of ATP binding function has been found on cells that are understood to be unable to undergo programmed cell death, such as preneoplastic cells and neoplastic cells. This isoform of the receptor has been referred to as a “non functional” receptor.

Antibodies generated from immunisation with a peptide including Pro210 in cis bind to non functional P2X₇ receptors. However, they do not bind to P2X₇ receptors capable of binding ATP. Accordingly, these antibodies are useful for selectively detecting many forms of carcinoma and haemopoietic cancers and to treatment of some of these conditions.

WO02/057306A1 and WO03/020762A1 both discuss a probe for distinguishing between functional P2X₇ receptors and non functional P2X₇ receptors in the form of a monoclonal antibody.

WO2009./033233 discusses an epitope present on non functional receptors but not functional receptors and antibodies for binding thereto.

To date it has been very difficult to obtain serological reagents that bind to non functional P2X₇ receptors on live cells with desirable affinity. Higher affinity reagents are generally desirable in applications for the detection and treatment of cancer.

There is a need for improved reagents for binding to P2X₇ receptors, particularly for new antibodies and fragments thereof that are capable of discriminating between ATP and non-ATP binding P2X₇ receptors on live cells.

### SUMMARY OF THE INVENTION

In one embodiment there is provided an antigen binding site for binding to a P2X₇ receptor, the antigen binding site being defined by general formula 1:

FR1-CDR1-FR2-CDR2-FR3-CDR3-FR4

wherein:
FR1, FR2, FR3 and FR4 are each framework regions;
CDR1, CDR2 and CDR3 are each complementarity determining regions;
   wherein:
CDR1 has a sequence selected from the group consisting of: DNEPMG, RNHDMG, SGYAMA, GMYNMS, PASNMS, GSYAMA, GAYAMS, DGYNMS, TYDMAW, QEYGMG, ARYPMA, SSYAMA, AKYPMV, SSYAMS, DNVEMS and PMKDMG.

In one embodiment there is provided an antigen binding site for binding to a P2X₇ receptor, the antigen binding site being defined by general formula 2:

FR1-CDR1-FR2-CDR2-FR3-CDR3-FR4

wherein:
FR1, FR2, FR3 and FR4 are each framework regions;
CDR1, CDR2 and CDR3 are each complementarity determining regions;
   wherein:

CDR2 has a sequence selected from the
group consisting of: SIADSGNHTYYADSVKG,
AISGSGGSTYYADSVKG, TILSDGSRTYYADSVKG,
SINATGGRTYYADSVKG, SITASGYRTYYADSVKG,
TISTSGSSTYYADSVKG, TINGSGLATYYADSVKG,
SITANGNSTYYADSVKG, SIAAAGSRTYYADSVKG,
SITPSGDKTYYADSVKG, SIDGGGLQTYYADSVKG,
TIDGNGLITYYADSVKG, SIGPGGARTYYADSVKG,
TITSDGLRTYYADSVKG, SIGSKGEDTYYADSVKG,
AISGSGGSTYYANSVKG, AISGSGGGTYYADSVKG,
SIGTKGEYTYYADSVKG, SIGSKGEYTYYADSVKG and
AISGSGGGTYYANSVKG.

In one embodiment there is provided an antigen binding site for binding to a P2X₇ receptor, the antigen binding site being defined by general formula 3:

FR1-CDR1-FR2-CDR2-FR3-CDR3-FR4

wherein:
FR1, FR2, FR3 and FR4 are each framework regions;
CDR1, CDR2 and CDR3 are each complementarity determining regions;
   wherein:

CDR3 has a sequence selected from the
group consisting of: KQRGLNRYRAQFDY, EPKPMDTEFDY,
KIKTFRNHSVQFDY, KFNGFSHRQYNFDY, KQGQISNFPRFDY,
KVRFATSKSINFDY, KCSSCTSLNANFDY, KASYSRPYNFQFDY,
KQRSISIRPMFDY, KVRSMSYAHFDFDY, KASAPKYFRFDY,
KLQRYDRYTLNFDY, KPWRVYSYDRFDY, KVHTFANRSLNFDY,
QTVNVPEPAFAY and EPSHFDRPFDY.

In one embodiment there is provided an antigen binding site for binding to a P2X₇ receptor, the antigen binding site being defined by general formula 4:

FR1-CDR1-FR2-CDR2-FR3-CDR3-FR4

wherein:
FR1, FR2, FR3 and FR4 are each framework regions;
CDR1, CDR2 and CDR3 are each complementarity determining regions;
   wherein:
CDR1 has a sequence selected from the group consisting of: (P/R)(N/M)(H/K)DMG.

In one embodiment there is provided an antigen binding site for binding to a P2X₇ receptor, the antigen binding site being defined by general formula 5:

FR1-CDR1-FR2-CDR2-FR3-CDR3-FR4

wherein:
FR1, FR2, FR3 and FR4 are each framework regions;
CDR1, CDR2 and CDR3 are each complementarity determining regions;
   wherein:
CDR2 has a sequence selected from the group consisting of:

AISGSGG(S/G)TYYA(D/N)SVKG.

In one embodiment there is provided an antigen binding site for binding to a P2X₇ receptor, the antigen binding site being defined by general formula 6:

FR1-CDR1-FR2-CDR2-FR3-CDR3-FR4

wherein:
FR1, FR2, FR3 and FR4 are each framework regions;
CDR1, CDR2 and CDR3 are each complementarity determining regions;
   wherein:
CDR3 has a sequence selected from the group consisting of:

EP(K/S)(P/H)(M/F)D(T/R)(E/P)FDY.

In one embodiment there is provided an antigen binding site for binding to a P2X₇ receptor, the antigen binding site being defined by general formula 7:

FR1-CDR1-FR2-CDR2-FR3-CDR3-FR4

wherein:
FR1, FR2, FR3 and FR4 are each framework regions;
CDR1, CDR2 and CDR3 are each complementarity determining regions;
   wherein:

CDR3 has a sequence: EP(K/S)(P/H)(M/F)D(T/R)
(E/P)FDY;
and
FR4 has a sequence: (W/R/P/G/C)(G/S/F)(Q/P/C)GT
(L/Q)VTV(S/L)(S/E).

In one embodiment there is provided an antigen binding site for binding to a P2X₇ receptor, the antigen binding site being defined by general formula 8:

FR1-CDR1-FR2-CDR2-FR3-CDR3-FR4

wherein:
FR1, FR2, FR3 and FR4 are each framework regions;
CDR1, CDR2 and CDR3 are each complementarity determining regions;
   wherein:

CDR1 has a sequence: (P/R)(N/M)(H/K)DMG;
CDR2 has a sequence: AISGSGG(S/G)TYYA(D/N)SVKG;
CDR3 has a sequence:
EP(K/S)(P/H)(M/F)D(T/R)(E/P)FDY;
and
FR4 has a sequence:
(W/R/P/G/C)(G/S/F)(Q/P/C)GT(L/Q)VTV(S/L)(S/E).

In one embodiment there is provided an antigen binding site for binding to a P2X₇ receptor, the antigen binding site being defined by general formula 9:

FR1-CDR1-FR2-CDR2-FR3-CDR3-FR4

wherein:
FR1, FR2, FR3 and FR4 are each framework regions;
CDR1, CDR2 and CDR3 are each complementarity determining regions;
   wherein:

CDR1 has a sequence: (P/R)(N/M)(H/K)DMG;
CDR2 has a sequence: AISGSGG(S/G)TYYA(D/N)SVKG;
CDR3 has a sequence:
EP(K/S)(P/H)(M/F)D(T/R)(E/P)FDY;
FR1 has a sequence: EVQLLE
(S/P)GGGLVQPGGSLRLSCAASG(Y/F/V)(R/T/N)(I/F/V);
FR2 has a sequence: W(V/A)RQAPGKGLEW(V/A)S;
FR3 has a sequence:
RFTISRDNS(R/K)NTLYLQMNS(L/M)RAEDTAVYYCA;
FR4 has a sequence:
(W/R/P/G/C)(G/S/F)(Q/P/C)GT(L/Q)VTV(S/L)(S/E).

In one embodiment there is provided an antigen binding site for binding to a P2X₇ receptor, the antigen binding site being defined by general formula 10:

FR1-CDR1-FR2-CDR2-FR3-CDR3-FR4

wherein:
FR1, FR2, FR3 and FR4 are each framework regions;
CDR1, CDR2 and CDR3 are each complementarity determining regions;
   wherein:

CDR1 has a sequence: PMKDMG;
CDR2 has a sequence: AISGSGGGTYYADSVKG;
CDR3 has a sequence: EPKPMDTEFDY;
FR1 has a sequence: EVQLLESGGGLVQPGGSLRLSCAASGYTF;
FR2 has a sequence: WVRQAPGKGLEWVS;
FR3 has a sequence:
RFTISRDNSKNTLYLQMNSLRAEDTAVYYCA;
FR4 has a sequence: PSPGTLVTVLE, WGQGTLVTVSS,
WGQGTLVTVLS, RSPGTLVTVSS, PSPGTQVTVSS,
PSPGTLVTVSS, RSQGTLVTVSS, WSQGTLVTVSS,
RGQGTLVTVSS, RFQGTLVTVSS, WSPGTLVTVSS,
GSPGTLVTVSS, WGPGTLVTVSS, RGPGTLVTVSS,
CGPGTLVTVSS, RSCGTLVTVSS, or RSPGTLVTVLE.

In other embodiments there is provided an antigen binding site having a sequence as described herein, or including a CDR and/or FR sequence as described herein and including one or more mutations for increasing the affinity of said site for binding to a P2X₇ receptor.

In another embodiment there is provided an antigen binding site as described herein wherein an amino acid sequence forming one or more of FR1, CDR1, FR2, CDR2, FR3, CDR3 and FR4 is a human sequence.

In another embodiment there is provided an anti P2X₇ receptor immunoglobulin variable domain, antibody, Fab, dab, scFv including an antigen binding site having a sequence as described herein, or including a CDR and/or FR sequence as described herein.

In another embodiment there is provided a diabody or triabody including an antigen binding site having a sequence as described herein, or including a CDR and/or FR sequence as described herein.

In another embodiment there is provided a fusion protein including an antigen binding site, immunoglobulin variable domain, antibody, Fab, dab, scFv, diabody or triabody as described herein.

In another embodiment there is provided a conjugate in the form of an antigen binding site, immunoglobulin variable domain, antibody, Fab, dab, scFv, diabody, triabody or fusion protein as described herein conjugated to a label or a cytotoxic agent.

In another embodiment there is provided an antibody for binding to an antigen binding site of an immunoglobulin variable domain, antibody, Fab, dab, scFv, diabody, triabody, fusion protein, or conjugate as described herein.

In another embodiment there is provided a nucleic acid encoding an antigen binding site, or a CDR and/or FR sequence as described herein, or an immunoglobulin variable domain, antibody, Fab, dab, scFv, diabody, triabody, fusion protein or conjugate as described herein.

In another embodiment there is provided a vector including a nucleic acid described herein.

In another embodiment there is provided a cell including a vector or nucleic acid described herein.

In another embodiment there is provided an animal or tissue derived therefrom including a cell described herein.

In another embodiment there is provided a pharmaceutical composition including an antigen binding site, or including a CDR and/or FR sequence as described herein, or an immunoglobulin variable domain, antibody, Fab, dab, scFv, diabody, triabody, fusion protein, or conjugate as described herein and a pharmaceutically acceptable carrier, diluent or excipient.

In another embodiment there is provided a diagnostic composition including an antigen binding site, or including a CDR and/or FR sequence as described herein, or an immunoglobulin variable domain, antibody, Fab, dab, scFv, diabody, triabody, fusion protein or conjugate as described herein, a diluent and optionally a label.

In another embodiment there is provided a kit or article of manufacture including an antigen binding site, or including a CDR and/or FR sequence as described herein or an immunoglobulin variable domain, antibody, Fab, dab, scFv, diabody, triabody, fusion protein or conjugate as described herein.

In another embodiment there is provided a use of a sequence according to one or more of CDR1, CDR2, FR1, FR2, FR3 and FR4 as described herein to produce an antigen binding site for binding to a P2X₇ receptor.

In another embodiment there is provided a use of an antigen binding site or a CDR and/or FR sequence as described herein to produce an anti P2X₇ receptor antigen binding site having increased affinity for P2X₇ receptor.

In another embodiment there is provided a library of nucleic acid molecules produced from the mutation of an antigen binding site or a CDR and/or FR sequence as described herein, wherein at least one nucleic acid molecule in said library encodes an antigen binding site for binding to an a P2X₇ receptor.

In another embodiment there is provided a method for producing an anti P2X₇ antigen binding site as described herein including expressing a nucleic acid as described herein in a cell or animal as described herein.

In another embodiment there is provided a method for the treatment of cancer or a condition or disease associated with expression of non functional P2X₇ receptor in an individual including the step of providing an antigen binding site, immunoglobulin variable domain, antibody, Fab, dab, scFv, diabody, triabody, fusion protein, conjugate or pharmaceutical composition as described herein to an individual requiring treatment for cancer or said condition or disease.

In another embodiment there is provided a use of an antigen binding site, immunoglobulin variable domain, antibody, Fab, dab, scFv, diabody, triabody, fusion protein, conjugate or pharmaceutical composition as described herein in the manufacture of a medicament for the treatment of cancer or a condition or disease associated with expression of non functional P2X₇ receptor.

In another embodiment there is provided a method for the diagnosis of cancer or disease or condition associated with expression of non functional P2X₇ receptor, including the step of contacting tissues or cells for which the presence or absence of cancer is to be determined with a reagent in the form of an antigen binding site, immunoglobulin variable domain, antibody, Fab, dab, scFv, diabody, triabody, fusion protein, conjugate or diagnostic composition as described herein and detecting for the binding of the reagent with the tissues or cells. The method may be operated in vivo or in vitro.

Typically the antigen binding sites according to the invention bind to non functional P2X₇ receptors, especially receptors wherein Pro210 of P2X₇ is in cis conformation. In certain embodiments the antigen binding sites according to the invention do not bind to functional P2X₇ receptors, especially receptors wherein Pro210 of P2X₇ is in trans conformation.

Typically the antigen binding sites according to the invention bind to non functional P2X₇ receptors on live cells. In other embodiments, the antigen binding site does not bind to receptors on dead or fixed cells tissues, such as those as studied in histology or cytology.

In one embodiment, the antigen binding sites according to the invention bind to P2X₇ receptors on live cells with affinities in the range of 0.1 to 5 nM.

In one embodiment, there is provided a single domain antibody including an antigen binding site for binding to a P2X7 receptor, preferably to a non functional P2X7 receptor.

### BRIEF DESCRIPTION OF THE DRAWINGS

FIG. 1. Round 2 dAb ELISA positives screened on Biacore from the Round 2 phage

FIG. 2. 20 nM PEP2-4, no peptide, cervical cancer, objective x10

FIG. 3. 20 nM PEP2-4, 0.1 mM peptide, cervical cancer, serial section, limited binding

FIG. 4. 20 nM PEP2-4, 1.0 mM peptide, cervical cancer, serial section, no binding

FIG. 5. 20 nM PEP2-4, no peptide, cervical cancer, objective x10

FIG. 6. 20 nM PEP2-4, 0.1 mM peptide, cervical cancer, serial section, limited binding

FIG. 7. 20 nM Pep2-4, 1.0 mM peptide, cervical cancer, serial section, no binding

FIG. 8. 20 nM Pep2-4, no peptide, cervical cancer

FIG. 9. 20 nM Pep2-4, 10 uM peptide, cervical cancer, serial section, binding unaffected

FIG. 10. 20 Nm PEP2-4 Melanoma, objective x20

FIG. 11. Lead dAb-Fc expressing at a molecular weight of 75kDa

FIG. 12. Traces that can be easily resolved from the bottom include the control dAb, HEL4-Fc (green), PEP2-47, PEP2-42, PEP2-42-1, PEP2-2 (blue) with other higher affinity binders above. Flow rate was 50 uL/min. FIG. 13. The P2X₇ extracellular domain 47-332 with C-terminal c-Myc tag and N-terminal HA tag attached to PDGFR transmembrane anchorage for use in screening E200 conformational antigen binders expressed on HEK293 cells.

FIG. 14. SDS-PAGE Western blot of cell lystate and surface expressed proteins expressing the ECD1, wild type(WT) P2X₇ and the two non-functional P2X₇ receptor mutants R307Q and E496A. The ECD1 is expressed at 52 kDa, the three P2X₇ receptors at 75 kDa. Anti-cadherin control in the lower section is at 98 kDa and anti-actin in the cell lysate at 42 kDa.

FIG. 15. SDS-PAGE of ECD2 (47-306) and a mutant construct K193A(47-306) showing protein A fractions 1-5 and the supernatant (NB) with molecular weight standards at left.

FIG. 16. SDS-PAGE both non-reduced and reduced of dAb-Fc and ECD2-Fc along with corresponding Western Blots reacted with anti-P2X₇ antibody.

FIG. 17. A selection of dAbs tested at 5 uM. Staining was detected with anti-human IgG Fab.

FIG. 18. Flow cytometry of the dAb-Fc binding to the pDisplay-ECD2 on HEK cells showing tighter cell binding by higher affinity species.

FIG. 19. Biacore tracings of selected PEP2-42 clones showing improved binding to E200 peptide.

FIG. 20. Sequences of PEP2-42 clones.

FIG. 21. Tree of affinity maturation pathways from lead binder to expressed extracellular domain of target receptor

FIG. 22. Biacore traces of the PEP2-2-3 Fc clone at increasing concentrations run against 10RU of E200 peptide.

FIG. 23. Biacore traces of clones produced by NNS screening of Trp103 in PEP2-2-1.

FIG. 24. Binding by flow cytometry of PEP2-2-1 to cells expressing ECD1 or ECD2 together with controls (mock and pDisplay only).

FIG. 25. Biacore tracings showing competitive binding between PEP2-2-1, E200 peptide and ECD2 (47-306).

FIG. 26. Lead dAbs 2-2-1 Fc, 2-2-3 Fc and 2-2-8 Fc binding to live prostate PC3 cells at 0-20 ug/mL.

FIG. 27. Lead dAbs 2-2-1 Fc, 2-2-3 Fc and 2-2-8 Fc binding to live breast MDA MB 231 cells at 0-20 ug/mL.

FIG. 28. Lead dAbs 2-2-1 Fc, 2-2-3 Fc and 2-2-8 Fc binding to live ovarian SKOV-3 cells at 0-20 ug/mL.

FIG. 29. Lead dAbs 2-2-1 Fc, 2-2-3 Fc and 2-2-8 Fc binding to live renal 786-O cells at 0-20 ug/mL.

FIG. 30. Lead dAbs 2-2-1 Fc, 2-2-3 Fc and 2-2-8 Fc binding to live melanoma G361 cells at 0-20 ug/mL.

FIG. 31. Lead dAbs 2-2-1 Fc, 2-2-3 Fc and 2-2-8 Fc binding to live lung NCI-H596 cells at 0-20 ug/mL.

FIG. 32. Flow cytometry of human lymphocytes and monocytes from PBMC showing no binding by PEP2-2-1 Fc or PEP2-2-3 Fc.

FIG. 33. Flow cytometry of prostate LNCap cells showing binding by PEP2-2-1 Fc, PEP2-2-3 Fc and HLA whereas the HEL4 control shows no binding above the secondary alone.

FIG. 34. CTB assay showing inhibition of PC3 cell growth over 5 days in the presence of increased PEP2-2-1 Fc and PEP2-2-3 Fc compared with control HEL4 Fc.

FIG. 35. CTB assay showing inhibition of COLO205 cell growth over 3 and 5 days in the presence of increased PEP2-2-1 Fc and PEP2-2-3 Fc compared with control HEL4 Fc.

FIG. 36. CTB assay showing inhibition of A375 cell growth over 3 and 5 days in the presence of increased PEP2-2-1 Fc and PEP2-2-3 Fc compared with control HEL4 Fc

FIG. 37. Biacore traces of PEP2-2-12 dAb domain tested at 10, 5, 2.5, 1 and 0.5 nM.

FIG. 38. Biacore traces of PEP2-2-12Alexa488 domain tested at 5 and 2.5 nM.

FIG. 39. Biacore traces of PEP2-472-12Alexa488 domain tested at 10 and 5 nM.

FIG. 40. Alignment of dAb sequences.

FIG. 41. (SEQ ID NO:1) Sequence of P2X₇.

FIG. 42. (SEQ ID NO:2) Sequence of ECD2

FIG. 43 (SEQ ID NO:3) Sequence of ECD1.

FIG. 44. Map of construct pcDNA3.1 PEP2-2-1 dAb-FC.

FIGS. 45A-C (SEQ ID NO: 198) Sequence of pcDNA3.1 PEP2-2-1 dAb-FC.

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described.

It will be understood that the invention disclosed and defined in this specification extends to all alternative combinations of two or more of the individual features mentioned or evident from the text or drawings. All of these different combinations constitute various alternative aspects of the invention.

As used herein, except where the context requires otherwise, the term “comprise” and variations of the term, such as “comprising”, “comprises” and “comprised”, are not intended to exclude further additives, components, integers or steps.

For purposes of interpreting this specification, the following definitions will apply and whenever appropriate, terms used in the singular will also include the plural and vice versa. In the event that any definition set forth conflicts with any document incorporated herein by reference, the definition set forth below shall prevail.

“Purinergic receptor” generally refers to a receptor that uses a purine (such as ATP) as a ligand.

“P2X₇ receptor” generally refers to a purinergic receptor formed from three protein subunits or monomers, with at least one of the monomers having an amino acid sequence substantially as shown in SEQ ID NO:1. “P2X₇ receptor” may be a functional or non functional receptor as described below. “P2X₇ receptor” encompasses naturally occurring variants of P2X₇ receptor, e.g., wherein the P2X₇ monomers are splice variants, allelic variants and isoforms including naturally-occurring truncated or secreted forms of the monomers forming the P2X₇ receptor (e.g., a form consisting of the extracellular domain sequence or truncated form of it), naturally-occurring variant forms (e.g., alternatively spliced forms) and naturally-occurring allelic variants. In certain embodiments of the invention, the native sequence P2X₇ monomeric polypeptides disclosed herein are mature or full-length native sequence polypeptides comprising the full-length amino acids sequence shown in SEQ ID NO:1. In certain embodiments the P2X₇ receptor may have an amino acid sequence that is modified, for example various of the amino acids in the sequence shown in SEQ ID NO:1 may be substituted, deleted, or a residue may be inserted.

“Functional P2X₇ receptor” generally refers to a form of the P2X₇ receptor having a binding site or cleft for binding to ATP. When bound to ATP, the receptor forms a pore-like structure that enables the ingress of calcium ions into the cytosol, one consequence of which may be programmed cell death. In normal homeostasis, expression of functional P2X₇ receptors is generally limited to cells that undergo programmed cell death such as thymocytes, dendritic cells, lymphocytes, macrophages and monocytes. There may also be some expression of functional P2X₇ receptors on erythrocytes.

“Non functional P2X₇ receptor” generally refers to a form of a P2X₇ receptor in which one or more of the monomers has a cis isomerisation at Pro210 (according to SEQ ID NO: 1). The isomerisation may arise from any molecular event that leads to misfolding of the monomer, including for example, mutation of monomer primary sequence or abnormal post translational processing. One consequence of the isomerisation is that the receptor is unable to bind to ATP, or otherwise binds ATP with a lower affinity than observed between ATP and receptors which do not contain an isomerisation at Pro210. In the circumstances, the receptor cannot form a pore and this limits the extent to which calcium ions may enter the cytosol. Non functional P2X₇ receptors are expressed on a wide range of epithelial and haematopoietic cancers.

“Extracellular domain” (ECD) used herein are P2X₇ receptor (47-306) (SEQ ID NO: 2) (ECD2) and P2X₇ receptor (47-332) (SEQ ID NO:3) (ECD1). P2X₇ receptor (47-306) (SEQ ID NO: 2) is amino acids 47 to 306 of SEQ ID NO: 1. P2X₇ receptor (47-332) (SEQ ID NO: 3) is amino acids 47 to 332 of SEQ ID NO: 1.

“Antibodies” or “immunoglobulins” or “Igs” are gamma globulin proteins that are found in blood, or other bodily fluids of verterbrates that function in the immune system to bind antigen, hence identifying and neutralizing foreign objects.

Antibodies are generally a heterotetrameric glycoprotein composed of two identical light (L) chains and two identical heavy (H) chains. Each L chain is linked to a H chain by one covalent disulfide bond. The two H chains are linked to each other by one or more disulfide bonds depending on the H chain isotype. Each H and L chain also has regularly spaced intrachain disulfide bridges.

H and L chains define specific Ig domains. More particularly, each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V _{L}) followed by a constant domain (C_{L}) at its other end. The V_{L} is aligned with the V_{H} and the C_{L} is aligned with the first constant domain of the heavy chain (C_{H}1).

Antibodies can be assigned to different classes or isotypes. There are five classes of immunoglobulins: IgA, IgD, IgE, IgG, and IgM, having heavy chains designated α, δ, ε, γ, and μ, respectively. The γ and α classes are further divided into subclasses on the basis of relatively minor differences in CH sequence and function, e.g., humans express the following subclasses: IgG1, IgG2, IgG3, IgG4, IgA1, and IgA2. The L chain from any vertebrate species can be assigned to one of two clearly distinct types, called kappa and lambda, based on the amino acid sequences of their constant domains.

The constant domain includes the Fc portion which comprises the carboxy-terminal portions of both H chains held together by disulfides. The effector functions of antibodies such as ADCC are determined by sequences in the Fc region, which region is also the part recognized by Fc receptors (FcR) found on certain types of cells.

The pairing of a V_{H} and V_{L} together forms a “variable region” or “variable domain” including the amino-terminal domains of the heavy or light chain of the antibody. The variable domain of the heavy chain may be referred to as “VH.” The variable domain of the light chain may be referred to as “VL.” The V domain contains an antigen binding site which affects antigen binding and defines specificity of a particular antibody for its particular antigen. V regions span about 110 amino acid residues and consist of relatively invariant stretches called framework regions (FRs) (generally about 4) of 15-30 amino acids separated by shorter regions of extreme variability called “hypervariable regions” (generally about 3) that are each 9-12 amino acids long. The FRs largely adopt a β-sheet configuration and the hypervariable regions form loops connecting, and in some cases forming part of, the β-sheet structure.

“Hypervariable region”, “HVR”, or “HV” refers to the regions of an antibody variable domain which are hypervariable in sequence and/or form structurally defined loops. Generally, antibodies comprise six hypervariable regions; three in the VH (HI, H2, H3), and three in the VL (LI, L2, L3). A number of hypervariable region delineations are in use and are encompassed herein. The Kabat Complementarity Determining Regions (CDRs) are based on sequence variability and are the most commonly used (Kabat et al, Sequences of Proteins of Immunological Interest, 5th Ed. Public Health Service, National Institutes of Health, Bethesda, MD. (1991)).

“Framework” or “FR” residues are those variable domain residues other than the hypervariable region residues herein defined.

“A peptide for forming an antigen binding site” generally refers to a peptide that may form a conformation that confers the specificity of an antibody for antigen. Examples include whole antibody or whole antibody related structures, whole antibody fragments including a variable domain, variable domains and fragments thereof, including light and heavy chains, or fragments of light and heavy chains that include some but not all of hypervariable regions or constant regions.

An “intact” or “whole” antibody is one which comprises an antigen-binding site as well as a C_{L} and at least heavy chain constant domains, C_{H}I, C_{H}2 and C_{H}3. The constant domains may be native sequence constant domains (e.g. human native sequence constant domains) or amino acid sequence variant thereof.

“Whole antibody related structures” include multimerized forms of whole antibody.

“Whole antibody fragments including a variable domain” include Fab, Fab′, F(ab′)₂, and Fv fragments; diabodies; linear antibodies, single-chain antibody molecules; and multispecific antibodies formed from antibody fragments.

The Fab fragment consists of an entire L chain along with the variable region domain of the H chain (V_{H}), and the first constant domain of one heavy chain (C_{H}I). Each Fab fragment is monovalent with respect to antigen binding, i.e., it has a single antigen-binding site.

A Fab′ fragment differs from Fab fragments by having additional few residues at the carboxy terminus of the C_{H}I domain including one or more cysteines from the antibody hinge region. Fa′-SH is the designation herein for Fab′ in which the cysteine residue(s) of the constant domains bear a free thiol group.

A F(ab′)₂ fragment roughly corresponds to two disulfide linked Fab fragments having divalent antigen-binding activity and is still capable of cross-linking antigen.

An “Fv” is the minimum antibody fragment which contains a complete antigen-recognition and -binding site. This fragment consists of a dimer of one heavy- and one light-chain variable region domain in tight, non-covalent association.

In a single-chain Fv (scFv) species, one heavy-and one light-chain variable domain can be covalently linked by a flexible peptide linker such that the light and heavy chains can associate in a “dimeric” structure analogous to that in a two-chain Fv species. From the folding of these two domains emanate six hypervariable loops (3 loops each from the H and L chain) that contribute the amino acid residues for antigen binding and confer antigen binding specificity to the antibody.

“Single-chain Fv” also abbreviated as “sFv” or “scFv” are antibody fragments that comprise the V_{H} and V_{L} antibody domains connected to form a single polypeptide chain. Preferably, the scFv polypeptide further comprises a polypeptide linker between the V_{H} and V_{L} domains which enables the scFv to form the desired structure for antigen binding.

A “single variable domain” is half of an Fv (comprising only three CDRs specific for an antigen) that has the ability to recognize and bind antigen, although at a lower affinity than the entire binding site

“Diabodies” refers to antibody fragments with two antigen-binding sites, which fragments comprise a heavy-chain variable domain (VH) connected to a light-chain variable domain (VL) in the same polypeptide chain (VH-VL). The small antibody fragments are prepared by constructing sFv fragments (see preceding paragraph) with short linkers (about 5-10 residues) between the V_{H} and V_{L} domains such that interchain but not intra-chain pairing of the V domains is achieved, resulting in a bivalent fragment, i.e., fragment having two antigen-binding sites.

Diabodies may be bivalent or bispecific. Bispecific diabodies are heterodimers of two “crossover” sFv fragments in which the V_{H} and V_{L} domains of the two antibodies are present on different polypeptide chains. Triabodies and tetrabodies are also generally know in the art.

An “isolated antibody” is one which has been identified and separated and/or recovered from a component of its pre-existing environment. Contaminant components are materials that would interfere with therapeutic uses for the antibody, and may include enzymes, hormones, and other proteinaceous or nonproteinaceous solutes.

A “human antibody” refers to an antibody which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Human antibodies can be prepared by administering the antigen to a transgenic animal that has been modified to produce such antibodies in response to antigenic challenge, but whose endogenous loci have been disabled.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin.

“Monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site or determinant on the antigen. In addition to their specificity, the monoclonal antibodies are advantageous in that they may be synthesized uncontaminated by other antibodies. Monoclonal antibodies may be prepared by the hybridoma methodology, or may be made using recombinant DNA methods in bacterial, eukaryotic animal or plant cells. The “monoclonal antibodies” may also be isolated from phage antibody libraries.

The monoclonal antibodies herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. Chimeric antibodies of interest herein include “primatized” antibodies comprising variable domain antigen-binding sequences derived from a non-human primate (e.g. Old World Monkey, Ape etc), and human constant region sequences.

The term “anti-P2X₇ receptor antibody” or “an antibody that binds to P2X₇ receptor” refers to an antibody that is capable of binding P2X₇ receptor with sufficient affinity such that the antibody is useful as a diagnostic and/or therapeutic agent in targeting P2X₇ receptor, typically non functional P2X₇ receptor. Preferably, the extent of binding of an P2X₇ receptor antibody to an unrelated receptor protein is less than about 10% of the binding of the antibody to P2X₇ receptor as measured, e.g., by a radioimmunoassay (RIA). In certain embodiments, an antibody that binds to P2X₇ receptor has a dissociation constant (Kd) of <1 μM, <100 nM, <10 nM, <1 nM, or <0.1 nM. An anti non functional P2X₇ receptor antibody is generally one having some or all of these serological characteristics and that binds to non functional P2X₇ receptors but not to functional P2X₇ receptors.

An “affinity matured” antibody is one with one or more alterations in one or more HVRs thereof which result in an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art.

A “blocking” antibody or an “antagonist” antibody is one which inhibits or reduces biological activity of the antigen it binds. Preferred blocking antibodies or antagonist antibodies substantially or completely inhibit the biological activity of the antigen.

An “agonist antibody”, as used herein, is an antibody which mimics at least one of the functional activities of a polypeptide of interest.

“Binding affinity” generally refers to the strength of the sum total of noncovalent interactions between a single binding site of a molecule (e.g., an antibody) and its binding partner (e.g., an antigen). Generally, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

The inventors have determined the CDR sequences of a number of variable domain clones that they have found to bind to the ECD target. These CDR sequences are shown in Table 1 below.

In one embodiment there is provided a peptide having a sequence as shown in Table 1. These peptides are particularly useful for constructing antigen binding sites, variable domains, antibodies and related fragments.

TABLE 1
CDR sequences

| Clone | CDR1 | CDR2 | CDR3 |
|---|---|---|---|
| PEP2-1 | SEQ ID NO: 4 | SEQ ID NO: 5 | SEQ ID NO: 6 |
| | DNEPMG | SIADSGNHTYYADSVKG | KQRGLNRYRAQFDY |
| PEP2-2 | SEQ ID NO: 7 | SEQ ID NO: 8 | SEQ ID NO: 9 |
| | RNHDMG | AISGSGGSTYYADSVKG | EPKPMDTEFDY |
| PEP2-3 | SEQ ID NO: 10 | SEQ ID NO: 11 | SEQ ID NO: 12 |
| | SGYAMA | TILSDGSRTYYADSVKG | KIKTFRNHSVQFDY |
| PEP2-4 | SEQ ID NO: 13 | SEQ ID NO: 14 | SEQ ID NO: 15 |
| | GMYNMS | SINATGGRTYYADSVKG | KFNGFSHRQYNFDY |
| PEP2-5 | SEQ ID NO: 16 | SEQ ID NO: 17 | SEQ ID NO: 18 |
| | PASNMS | SITASGYRTYYADSVKG | KQGQISNFPRFDY |
| PEP2-6 | SEQ ID NO: 19 | SEQ ID NO: 20 | SEQ ID NO: 21 |
| | GSYAMA | TISTSGSSTYYADSVKG | KVRFATSKSINFDY |
| PEP2-7 | SEQ ID NO: 22 | SEQ ID NO: 23 | SEQ ID NO: 24 |
| | GAYAMS | TINGSGLATYYADSVKG | KCSSCTSLNANFDY |
| PEP2-8 | SEQ ID NO: 25 | SEQ ID NO: 26 | SEQ ID NO: 27 |
| | DGYNMS | SITANGNSTYYADSVKG | KASYSRPYNFQFDY |
| PEP2-9 | SEQ ID NO: 28 | SEQ ID NO: 29 | SEQ ID NO: 30 |
| | TYDMAW | SIAAAGSRTYYADSVKG | KQRSISIRPMFDY |
| PEP2-10 | SEQ ID NO: 31 | SEQ ID NO: 32 | SEQ ID NO: 33 |
| | QEYGMG | SITPSGDKTYYADSVKG | KVRSMSYAHFDFDY |
| PEP2-11 | SEQ ID NO: 34 | SEQ ID NO: 35 | SEQ ID NO: 36 |
| | ARYPMA | SIDGGGLQTYYADSVKG | KASAPKYFRFDY |
| PEP2-13 | SEQ ID NO: 37 | SEQ ID NO: 38 | SEQ ID NO: 39 |
| | SSYAMA | TIDGNGLITYYADSVKG | KLQRYDRYTLNFDY |
| PEP2-30 | SEQ ID NO: 40 | SEQ ID NO: 41 | SEQ ID NO: 42 |
| | AKYPMV | SIGPGGARTYYADSVKG | KPWRVYSYDRFDY |
| PEP2-34 | SEQ ID NO: 43 | SEQ ID NO: 44 | SEQ ID NO: 45 |
| | SSYAMS | TITSDGLRTYYADSVKG | KVHTFANRSLNFDY |
| PEP2-42 | SEQ ID NO: 46 | SEQ ID NO: 47 | SEQ ID NO: 48 |
| | DNVEMS | SIGSKGEDTYYADSVKG | QTVNVPEPAFAY |
| PEP2-47 | SEQ ID NO: 49 | SEQ ID NO: 50 | SEQ ID NO: 51 |
| | PMKDMG | AISGSGGSTYYADSVKG | EPSHFDRPFDY |
| PEP2-2-1 | SEQ ID NO: 52 | SEQ ID NO: 53 | SEQ ID NO: 54 |
| | RNHDMG | AISGSGGSTYYANSVKG | EPKPMDTEFDY |
| PEP2-2-1-1 | SEQ ID NO: 55 | SEQ ID NO: 56 | SEQ ID NO: 57 |
| | RNHDMG | AISGSGGSTYYADSVKG | EPKPMDTEFDY |
| PEP2-2-1-2 | SEQ ID NO: 58 | SEQ ID NO: 59 | SEQ ID NO: 60 |
| | RNHDMG | AISGSGGSTYYADSVKG | EPKPMDTEFDY |
| PEP2-2-11 | SEQ ID NO: 61 | SEQ ID NO: 62 | SEQ ID NO: 63 |
| | RNHDMG | AISGSGGSTYYANSVKG | EPKPMDTEFDY |
| PEP2-2-12 | SEQ ID NO: 64 | SEQ ID NO: 65 | SEQ ID NO: 66 |
| | RNHDMG | AISGSGGSTYYANSVKG | EPKPMDTEFDY |
| PEP2-2-2 | SEQ ID NO: 67 | SEQ ID NO: 68 | SEQ ID NO: 69 |
| | RNHDMG | AISGSGGSTYYADSVKG | EPKPMDTEFDY |
| PEP2-2-4 | SEQ ID NO: 70 | SEQ ID NO: 71 | SEQ ID NO: 72 |
| | RNHDMG | AISGSGGSTYYADSVKG | EPKPMDTEFDY |
| PEP2-2-5 | SEQ ID NO: 73 | SEQ ID NO: 74 | SEQ ID NO: 75 |
| | RNHDMG | AISGSGGSTYYADSVKG | EPKPMDTEFDY |
| PEP2-2-8 | SEQ ID NO: 76 | SEQ ID NO: 77 | SEQ ID NO: 78 |
| | RNHDMG | AISGSGGSTYYADSVKG | EPKPMDTEFDY |
| PEP2-2-9 | SEQ ID NO: 79 | SEQ ID NO: 80 | SEQ ID NO: 81 |
| | RNHDMG | AISGSGGSTYYADSVKG | EPKPMDTEFDY |
| PEP2-2-81 | SEQ ID NO: 82 | SEQ ID NO: 83 | SEQ ID NO: 84 |
| | RNHDMG | AISGSGGSTYYADSVKG | EPKPMDTEFDY |
| PEP2-2-91 | SEQ ID NO: 85 | SEQ ID NO: 86 | SEQ ID NO: 87 |
| | RNHDMG | AISGSGGSTYYADSVKG | EPKPMDTEFDY |
| PEP2-2-3 | SEQ ID NO: 88 | SEQ ID NO: 89 | SEQ ID NO: 90 |
| | RNHDMG | AISGSGGSTYYADSVKG | EPKPMDTEFDY |
| PEP2-2-31 | SEQ ID NO: 91 | SEQ ID NO: 92 | SEQ ID NO: 93 |
| | RNHDMG | AISGSGGSTYYADSVKG | EPKPMDTEFDY |
| PEP2-2-32 | SEQ ID NO: 94 | SEQ ID NO: 95 | SEQ ID NO: 96 |
| | RNHDMG | AISGSGGSTYYADSVKG | EPKPMDTEFDY |
| PEP2-2-33 | SEQ ID NO: 97 | SEQ ID NO: 98 | SEQ ID NO: 99 |
| | RNHDMG | AISGSGGSTYYADSVKG | EPKPMDTEFDY |
| PEP2-2-10 | SEQ ID NO: 100 | SEQ ID NO: 101 | SEQ ID NO: 102 |
| | RNHDMG | AISGSGGSTYYADSVKG | EPKPMDTEFDY |
| PEP2-2-101 | SEQ ID NO: 103 | SEQ ID NO: 104 | SEQ ID NO: 105 |
| | RNHDMG | AISGSGGSTYYADSVKG | EPKPMDTEFDY |
| PEP2-2-102 | SEQ ID NO: 106 | SEQ ID NO: 107 | SEQ ID NO: 108 |
| | RNHDMG | AISGSGGSTYYADSVKG | EPKPMDTEFDY |
| PEP2-247-1 | SEQ ID NO: 109 | SEQ ID NO: 110 | SEQ ID NO: 111 |
| | RNHDMG | AISGSGGGTYYADSVKG | EPSHFDRPFDY |
| PEP2-247-2 | SEQ ID NO: 112 | SEQ ID NO: 113 | SEQ ID NO: 114 |
| | RNHDMG | AISGSGGSTYYANSVKG | EPSHFDRPFDY |
| PEP2-472-1 | SEQ ID NO: 115 | SEQ ID NO: 116 | SEQ ID NO: 117 |
| | PMKDMG | AISGSGGGTYYADSVKG | EPKPMDTEFDY |
| PEP2-472-11 | SEQ ID NO: 118 | SEQ ID NO: 119 | SEQ ID NO: 120 |
| | PMKDMG | AISGSGGGTYYADSVKG | EPKPMDTEFDY |
| PEP2-472-12 | SEQ ID NO: 121 | SEQ ID NO: 122 | SEQ ID NO: 123 |
| | PMKDMG | AISGSGGGTYYADSVKG | EPKPMDTEFDY |
| PEP2-472-121 | SEQ ID NO: 124 | SEQ ID NO: 125 | SEQ ID NO: 126 |
| | PMKDMG | AISGSGGGTYYADSVKG | EPKPMDTEFDY |
| PEP2-42-1 | SEQ ID NO: 127 | SEQ ID NO: 128 | SEQ ID NO: 129 |
| | DNVEMS | SIGTKGEYTYYADSVKG | QTVNVPEPAFAY |
| PEP2-42-2 | SEQ ID NO: 130 | SEQ ID NO: 131 | SEQ ID NO: 132 |
| | DNVEMS | SIGSKGEYTYYADSVKG | QTVNVPEPAFAY |
| PEP2-47-1 | SEQ ID NO: 133 | SEQ ID NO: 134 | SEQ ID NO: 135 |
| | PMKDMG | AISGSGGGTYYADSVKG | EPSHFDRPFDY |
| PEP2-47-2 | SEQ ID NO: 136 | SEQ ID NO: 137 | SEQ ID NO: 138 |
| | PMKDMG | AISGSGGGTYYANSVKG | EPSHFDRPFDY |

The inventors have determined the FR sequences of a number of variable domain clones that they have found to bind to the ECD target. These FR sequences are shown in Table 2 below. Other known FR sequences could be used with the above described CDRs to form an antigen binding site for binding to a non functional P2X₇ receptor.

TABLE 2
Framework regions

| Clone | FR1 |
|---|---|
| PEP2-1, PEP2-2, PEP2-3, PEP2-4, PEP2-5, PEP2-6, | SEQ ID NO: 139 |
| PEP2-7, PEP2-8, PEP2-10, PEP2-11, PEP2-13, PEP2- | EVQLLESGGGLVQPGGSLRLSCAASGFTF |
| 30, PEP2-34, PEP2-42, PEP2-47, PEP2-2-1, PEP2-2-1- | |
| 1, PEP2-2-1-2, PEP2-2-11, PEP2-2-12, PEP2-2-2, PEP2- | |
| 2-8, PEP2-2-9, PEP2-2-81, PEP2-2-91, PEP2-2-3, PEP2- | |
| 2-31, PEP2-2-32, PEP2-2-33, PEP2-2-10, PEP2-2-101, | |
| PEP2-2-102, PEP2-247-1, PEP2-247-2, PEP2-42, PEP2- | |
| 42-1; PEP2-2-5 | |
| HEL-4 | SEQ ID NO: 140 |
| | EVQLLESGGGLVQPGGSLRLSCAASGFRI |
| PEP2-9 | SEQ ID NO: 141 |
| | EVQLLESGGGLVQPGGSLRLSCAASGFTL |
| PEP2-47-1, PEP2-47-2, PEP2-472-1, PEP2-472-11, | SEQ ID NO: 142 |
| PEP2-472-12; PEP2-472-121 | EVQLLESGGGLVQPGGSLRLSCAASGYTF |
| PEP2-2-4 | SEQ ID NO: 143 |
| | EVQLLESGGGLVQPGGSLRLTCAASGFSF |
| PEP2-42-2 | SEQ ID NO: 144 |
| | EVQMLESGGGLVQPGESLRLSCAASGFTF |
| Clone | FR2 |
| PEP2-1, PEP2-2, PEP2-4, PEP2-5, PEP2-6, PEP2-7, | SEQ ID NO: 145 |
| PEP2-9, PEP2-10, PEP2-11, PEP2-13, PEP2-30, PEP2- | WVRQAPGKGLEWVS |
| 34, PEP2-42, PEP2-47, PEP2-2-1, PEP2-2-1-1, PEP2-2- | |
| 1-2, PEP2-2-11, PEP2-2-12, PEP2-2-2, PEP2-2-4, PEP2- | |
| 2-5, PEP2-2-8, PEP2-2-9, PEP2-2-81, PEP2-2-91, PEP2- | |
| 2-3, PEP2-2-31, PEP2-2-32, PEP2-2-33, PEP2-2-10, | |
| PEP2-2-101, PEP2-2-102, PEP2-472-1, PEP2-472-11, | |
| PEP2-472-12, PEP2-472-121, PEP2-247-1, PEP2-247-2, | |
| PEP2-42-1, PEP2-42-2, PEP2-47-1, PEP2-47-2 | |
| PEP2-3 | SEQ ID NO: 146 |
| | WVRQAPGKGLEWAS |
| PEP2-8 | SEQ ID NO: 147 |
| | WARQAPGKGLEWVS |
| Clone | FR3 |
| PEP2-1, PEP2-2, PEP2-3, PEP2-4, PEP2-5, PEP2-6, | SEQ ID NO: 148 |
| PEP2-7, PEP2-8, PEP2-9, PEP2-10, PEP2-11, PEP2- | RFTISRDNSKNTLYLQMNSLRAEDTAVYYCA |
| 13, PEP2-30, PEP2-42, PEP2-47, PEP2-2-1, PEP2-2-1- | |
| 1, PEP2-2-1-2, PEP2-2-11, PEP2-2-12, PEP2-2-2, | |
| PEP2-2-4, PEP2-2-8, PEP2-2-9, PEP2-2-81, PEP2-2- | |
| 91, PEP2-2-3, PEP2-2-31, PEP2-2-32, PEP2-2-33, | |
| PEP2-2-10, PEP2-2-101, PEP2-2-102, PEP2-472-1, | |
| PEP2-472-11, PEP2-472-12, PEP2-472-121, PEP2-247- | |
| 1, PEP2-247-2, PEP-2-47-1, PEP-2-47-2 | |
| PEP2-34 | SEQ ID NO: 149 |
| | RFTISRDNSRNTLYLQMNSLRAEDTAVYYCA |
| PEP2-42-1 | SEQ ID NO: 150 |
| | RFTISRDNSKNTLYLQMNSMRAEDTAVYYCA |
| PEP2-42-2 | SEQ ID NO: 151 |
| | RFTISRDNSKNTLYLQMNSPRAEDTAVYYCA |
| PEP2-2-5 | SEQ ID NO: 152 |
| | RFTISRDDSKNTLYLQMNSLRAEDTAVYYCA |
| Clone | FR4 |
| PEP2-1, PEP2-2, PEP2-3, PEP2-4, PEP2-5, PEP2-6, | SEQ ID NO: 153 |
| PEP2-7, PEP2-8, PEP2-9, PEP2-10, PEP2-11, PEP2-13, | WGQGTLVTVSS |
| PEP2-30, PEP2-34, PEP2-42, PEP2-47, PEP2-42-2 | |
| PEP2-42-1 | SEQ ID NO: 154 |
| | WGQGTLVTVLS |
| PEP2-2-1, PEP2-2-1-1, PEP2-2-32, PEP2-2-4, PEP2-2-5 | SEQ ID NO: 155 |
| | RSPGTLVTVSS |
| PEP2-2-11 | SEQ ID NO: 156 |
| | PSPGTQVTVSS |
| PEP2-2-12, PEP2-2-31 | SEQ ID NO: 157 |
| | PSPGTLVTVSS |
| PEP2-2-2, PEP2-47-1, PEP2-47-2, PEP2-472-1, PEP2- | SEQ ID NO: 158 |
| 247-1, PEP2-247-2 | RSQGTLVTVSS |
| PEP2-2-8, PEP2-2-81 | SEQ ID NO: 159 |
| | WSQGTLVTVSS |
| PEP2-2-9, | SEQ ID NO: 160 |
| | RGQGTLVTVSS |
| PEP2-2-91 | SEQ ID NO: 161 |
| | RFQGTLVTVSS |
| PEP2-2-3 | SEQ ID NO: 162 |
| | WSPGTLVTVSS |
| PEP2-2-33 | SEQ ID NO: 163 |
| | GSPGTLVTVSS |
| PEP2-2-10 | SEQ ID NO: 164 |
| | WGPGTLVTVSS |
| PEP2-2-101 | SEQ ID NO: 165 |
| | RGPGTLVTVSS |
| PEP2-2-102 | SEQ ID NO: 166 |
| | CGPGTLVTVSS |
| PEP2-472-11 | SEQ ID NO: 167 |
| | RSCGTLVTVSS |
| PEP2-472-12 | SEQ ID NO: 168 |
| | RSPGTLVTVLE |
| PEP2-472-121 | SEQ ID NO: 169 |
| | PSPGTLVTVLE |
| PEP2-2-1-2 | SEQ ID NO: 170 |
| | RSQGTLVTVSS |

In certain embodiments there is provided an antigen binding site having a sequence shown in Table 3 below:

TABLE 3
Antigen binding sites

| Clone | Antigen binding site sequence |
|---|---|
| PEP2-2 | SEQ ID NO: 171 |
| | PEP2-2 |
| | EVQLLESGGGLVQPGGSLRLSCAASGFRIRNHDMGWVRQAPGKGLEWVSAISGSGGSTYYADSVKGRFTISRDNSKNTLYLQMNSLRAEDTAVYYC |
| | AEPKPMDTE-FDYWGQGTLVTVSS |
| PEP2- | SEQ ID NO: 172 |
| 42 | PEP2-42 |
| | EVQLLESGGGLVQPGGSLRLSCAASGFTFDNVEMSWVRQAPGKGLEWVSSIGSKGEDTYYADSVKGRFTISRDNSKNTLYLQMNSLRAEDTAVY |
| | YCAQTVNVPEPAFAYWGQGTLVTVSS |
| PEP2- | SEQ ID NO: 173 |
| 47 | PEP2-47 |
| | EVQLLESGGGLVQPGGSLRLSCAASGFTFPMKDMGWVRQAPGKGLEWVSAISGSGGSTYYADSVKGRFTISRDNSKNTLYLQMNSLRAEDTAVY |
| | YCAEPSHFDRP-FDYWGQGTLVTVSS |
| PEP2- | SEQ ID NO: 174 |
| 2-1 | PEP2-2-1 |
| | EVQLLESGGGLVQPGGSLRLSCAASGFTFRNHDMGWVRQAPGKGLEWVSAISGSGGSTYYANSVKGRFTISRDNSKNTLYLQMNSLRAEDTAVY |
| | YCAEPKPMDTE-FDYRSPGTLVTVSS |
| PEP2- | SEQ ID NO: 175 |
| 2-1-1 | PEP2-2-1-1 |
| | EVQLLESGGGLVQPGGSLRLSCAASGFTFRNHDMGWVRQAPGKGLEWVSAISGSGGSTYYADSVKGRFTISRDNSKNTLYLQMNSLRAEDTAVY |
| | YCAEPKPMDTE-FDYRSPGTLVTVSS |
| PEP2- | SEQ ID NO: 176 |
| 2-1-2 | PEP2-2-1-2 |
| | EVQLLESGGGLVQPGGSLRLSCAASGFTFRNHDMGWVRQAPGKGLEWVSAISGSGGSTYYADSVKGRFTISRDNSKNTLYLQMNSLRAEDTAV |
| | YYCAEPKPMDTE-FDYRSQGTLVTVSS |
| PEP2- | SEQ ID NO: 177 |
| 2-11 | PEP2-2-11 |
| | EVQLLESGGGLVQPGGSLRLSCAASGFTFRNHDMGWVRQAPGKGLEWVSAISGSGGSTYYANSVKGRFTISRDNSKNTLYLQMNSLRAEDTAV |
| | YYCAEPKPMDTE-FDYPSPGTQVTVSS |
| PEP2- | SEQ ID NO: 178 |
| 2-12 | PEP2-2-12 |
| | EVQLLESGGGLVQPGGSLRLSCAASGFTFRNHDMGWVRQAPGKGLEWVSAISGSGGSTYYANSVKGRFTISRDNSKNTLYLQMNSLRAEDTAV |
| | YYCAEPKPMDTE-FDYPSPGTLVTVSS |
| PEP2- | SEQ ID NO: 179 |
| 2-2 | PEP2-2-2 |
| | EVQLLESGGGLVQPGGSLRLSCAASGFTFRNHDMGWVRQAPGKGLEWVSAISGSGGSTYYADSVKGRFTISRDNSKNTLYLQMNSLRAEDTAV |
| | YYCAEPKPMDTE-FDYRSQGTLVTVSS |
| PEP2- | SEQ ID NO: 180 |
| 2-4 | PEP2-2-4 |
| | EVQLLESGGGLVQPGGSLRLTCAASGFSFRNHDMGWVRQAPGKGLEWVSAISGSGGSTYYADSVKGRFTISRDNSKNTLYLQMNSLRAEDTAV |
| | YYCAEPKPMDTE-FDYRSPGTLVTVSS |
| PEP2- | SEQ ID NO: 181 |
| 2-5 | PEP2-2-5 |
| | EVQLLESGGGLVQPGGSLRLSCAASGFTFRNHDMGWVRQAPGKGLEWVSAISGSGGSTYYADSVKGRFTISRDDSKNTLYLQMNSLRAEDTAV |
| | YYCAEPKPMDTE-FDYRSPGTLVTVSS |
| PEP2- | SEQ ID NO: 182 |
| 2-8 | PEP2-2-8 |
| | EVQLLESGGGLVQPGGSLRLSCAASGFTFRNHDMGWVRQAPGKGLEWVSAISGSGGSTYYADSVKGRFTISRDNSKNTLYLQMNSLRAEDTAV |
| | YYCAEPKPMDTE-FDYFSQGTLVTVSS |
| PEP2- | SEQ ID NO: 183 |
| 2-9 | PEP2-2-9 |
| | EVQLLESGGGLVQPGGSLRLSCAASGFTFRNHDMGWVRQAPGKGLEWVSAISGSGGSTYYADSVKGRFTISRDNSKNTLYLQMNSLRAEDTAV |
| | YYCAEPKPMDTE-FDYRFQGTLVTVSS |
| PEP2- | SEQ ID NO: 184 |
| 2-3 | PEP2-2-3 |
| | EVQLLESGGGLVQPGGSLRLSCAASGFTFRNHDMGWVRQAPGKGLEWVSAISGSGGSTYYADSVKGRFTISRDNSKNTLYLQMNSLRAEDTAV |
| | YYCAEPKPMDTE-FDYWSPGTLVTVSS |
| PEP2- | SEQ ID NO: 185 |
| 2-10 | PEP2-2-10 |
| | EVQLLESGGGLVQPGGSLRLSCAASGFTFRNHDMGWVRQAPGKGLEWVSAISGSGGSTYYADSVKGRFTISRDNSKNTLYLQMNSLRAEDTAV |
| | YYCAEPKPMDTE-FDYRFPGTLVTVSS |
| PEP2- | SEQ ID NO: 186 |
| 2-101 | PEP2-2-101 |
| | EVQLLESGGGLVQPGGSLRLSCAASGFTFRNHDMGWVRQAPGKGLEWVSAISGSGGSTYYADSVKGRFTISRDNSKNTLYLQMNSLRAEDTAV |
| | YYCAEPKPMDTE-FDYRGPGTLVTVSS |
| PEP2- | SEQ ID NO: 187 |
| 2-102 | PEP2-2-102 |
| | EVQLLESGGGLVQPGGSLRLSCAASGFTFRNHDMGWVRQAPGKGLEWVSAISGSGGSTYYADSVKGRFTISRDNSKNTLYLQMNSLRAEDTAV |
| | YYCAEPKPMDTE-FDYCGPGTLVTVSS |
| PEP2- | SEQ ID NO: 188 |
| 472-1 | PEP2-472-1 |
| | EVQLLESGGGLVQPGGSLRLSCAASGYTFPMKDMGWVRQAPGKGLEWVSAISGSGGGTYYADSVKGRFTISRDNSKNTLYLQMNSLRAEDTAV |
| | YYCAEPKPMDTE-FDYRSQGTLVTVSS |
| PEP2- | SEQ ID NO: 189 |
| 472- | P2-472-11 |
| 11 | EVQLLESGGGLVQPGGSLRLSCAASGYTFPMKDMGWVRQAPGKGLEWVSAISGSGGGTYYADSVKGRFTISRDNSKNTLYLQMNSLRAEDTAV |
| | YYCAEPKPMDTE-FDYRSCGTLVTVSS |
| PEP2- | SEQ ID NO: 190 |
| 472- | P2-472-12 |
| 12 | EVQLLESGGGLVQPGGSLRLSCAASGYTFPMKDMGWVRQAPGKGLEWVSAISGSGGGTYYADSVKGRFTISRDNSKNTLYLQMNSLRAEDTAV |
| | YYCAEPKPMDTE-FDYRSPGTLVTVSS |
| PEP2- | SEQ ID NO: 191 |
| 472- | P2-472-121 |
| 121 | EVQLLESGGGLVQPGGSLRLSCAASGYTFPMKDMGWVRQAPGKGLEWVSAISGSGGGTYYADSVKGRFTISRDNSKNTLYLQMNSLRAEDTAV |
| | YYCAEPKPMDTE-FDYPSPGTLVTVSS |
| PEP2- | SEQ ID NO: 192 |
| 247-1 | PEP2-247-1 |
| | EVQLLESGGGLVQPGGSLRLSCAASGFTFRNHDMGWVRQAPGKGLEWVSAISGSGGGTYYADSVKGRFTISRDNSKNTLYLQMNSLRAEDTAV |
| | YYCAEPSHFDRP-FDYRSQGTLVTVSS |
| PEP2- | SEQ ID NO: 193 |
| 247-2 | PEP2-247-2 |
| | EVQLLESGGGLVQPGGSLRLSCAASGFTFRNHDMGWVRQAPGKGLEWVSAISGSGGSTYYANSVKGRFTISRDNSKNTLYLQMNSLRAEDTAV |
| | YYCAEPSHFDRP-FDYRSQGTLVTVSS |
| PEP2- | SEQ ID NO: 194 |
| 42-1 | PEP2-42-1 |
| | EVQMLESGGGLVQPGGSLRLSCAASGFTFDNVEMSWVRQAPGKGLEWVSSIGTKGEYTYYADSVKGRFTISRDNSKNTLYLQMNSMRAEDTAV |
| | YYCAQTVNVPEPAFAYWGQGTLVTVLS |
| PEP2- | SEQ ID NO: 195 |
| 42-2 | PEP2-42-2 |
| | EVQMLESGGGLVQPGESLRLSCAASGFTFDNVEMSWVRQAPGKGLEWVSSIGSKGEYTYYADSVKGRFTISRDNSKNTLYLQMNSPRAEDTAV |
| | YYCAQTVNVPEPAFAYWGQGTLVTVSS |
| PEP2- | SEQ ID NO: 196 |
| 47-1 | PEP2-47-1 |
| | EVQLLESGGGLVQPGGSLRLSCAASGYTFPMKDMGWVRQAPGKGLEWVSAISGSGGGTYYADSVKGRFTISRDNSKNTLYLQMNSLRAEDTAV |
| | YYCAEPSHFDRP-FDYRSQGTLVTVSS |
| PEP2- | SEQ ID NO: 197 |
| 47-2 | PEP2-47-2 |
| | EVQLLESGGGLVQPGGSLRLSCAASGYTFPMKDMGWVRQAPGKGLEWVSAISGSGGGTYYANSVKGRFTISRDNSKNTLYLQMNSLRAEDTAV |
| | YYCAEPSHFDRP-FDYRSQGTLVTVSS |

In one embodiment there is provided an antigen binding site for binding to a P2X₇ receptor, the antigen binding site being defined by general formula 11:

FR1-CDR1-FR2-CDR2-FR3-CDR3-FR4

wherein:
FR1, FR2, FR3 and FR4 are each framework regions;
CDR1, CDR2 and CDR3 are each complementarity determining regions;
   wherein:
CDR1 has a sequence selected from the group consisting of:

(R/P/D)(N/M)(H/K/V)(D/E)M(G/S)

In one embodiment there is provided an antigen binding site for binding to a P2X₇ receptor, the antigen binding site being defined by general formula 12:

FR1-CDR1-FR2-CDR2-FR3-CDR3-FR4

wherein:
FR1, FR2, FR3 and FR4 are each framework regions;
CDR1, CDR2 and CDR3 are each complementarity determining regions;
   wherein:
CDR2 has a sequence selected from the group consisting of:

(A/S)|(S/G)(G/S/T)(S/K)G(G/E)(S/G/D/Y)TYYA(D/
N)SVKG.

In one embodiment there is provided an antigen binding site for binding to a P2X₇ receptor, the antigen binding site being defined by general formula 13:

FR1-CDR1-FR2-CDR2-FR3-CDR3-FR4

wherein:
FR1, FR2, FR3 and FR4 are each framework regions;
CDR1, CDR2 and CDR3 are each complementarity determining regions;
   wherein:
CDR3 has a sequence selected from the group consisting of:

(E/Q)(P/T)(K/S/V)(P/H/N)(M/F/V)(D/P)(T/R/E)(E/
P)(A¹)F(A/D)Y

wherein A1 refers to no amino acid between (E/P) and F or alanine.

In one embodiment there is provided an antigen binding site for binding to a P2X₇ receptor, the antigen binding site being defined by general formula 14:

FR1-CDR1-FR2-CDR2-FR3-CDR3-FR4

wherein:
FR1, FR2, FR3 and FR4 are each framework regions;
CDR1, CDR2 and CDR3 are each complementarity determining regions;
   wherein:

CDR3 has a sequence: (E/Q)(P/T)(K/S/V)(P/H/N)(M/F/
V)(D/P)(T/R/E)(E/P)(A¹)F(A/D)Y
and
FR4 has a sequence: (W/R/P/G/C)(G/S/F)(Q/C/P)GT(L/
Q)VTV(S/L)(S/E).

In one embodiment there is provided an antigen binding site for binding to a P2X₇ receptor, the antigen binding site being defined by general formula 15:

FR1-CDR1-FR2-CDR2-FR3-CDR3-FR4

wherein:
FR1, FR2, FR3 and FR4 are each framework regions;
CDR1, CDR2 and CDR3 are each complementarity determining regions;
   wherein:

CDR1 has a sequence:
(R/P/D)(N/M)(H/K/V)(D/E)M(G/S);
CDR2 has a sequence:
(A/S)|(S/G)(G/S/T)(S/K)G(G/E)(S/G/D/Y)TYYA(D/N)
SVKG;
CDR3 has a sequence:
(E/Q)(P/T)(K/S/V)(P/H/N)(M/F/V)(D/P)(T/R/E)(E/P)
(A¹)F(A/D)Y;

wherein A¹ refers to no amino acid between (E/P) and F or alanine.
   and

FR4 has a sequence:
(W/R/P/G/C)(G/S/F)(Q/C/P)GT(L/Q)VTV(S/L)(S/E).

In one embodiment there is provided an antigen binding site for binding to a P2X₇ receptor, the antigen binding site being defined by general formula 16:

FR1-CDR1-FR2-CDR2-FR3-CDR3-FR4

wherein:
FR1, FR2, FR3 and FR4 are each framework regions;
CDR1, CDR2 and CDR3 are each complementarity determining regions;
   wherein:

CDR1 has a sequence:
(R/P/D)(N/M)(H/K/V)(D/E)M(G/S);
CDR2 has a sequence:
(A/S)|(S/G)(G/S/T)(S/K)G(G/E)(S/G/D/Y)TYYA(D/N)
SVKG;
CDR3 has a sequence:
(E/Q)(P/T)(K/S/V)(P/H/N)(M/F/V)(D/P)(T/R/E)(E/P)
(A¹)F(A/D)Y,

wherein A¹ refers to no amino acid between (E/P) and F or alanine;

FR1 has a sequence:
EVQLLE(S/P)GGGLVQPGGSLRLSCAASG(Y/F/V)(R/T/N)
(I/F/V);
FR2 has a sequence:
WVRQAPGKGLEWVS;
FR3 has a sequence:
RFTISRDNSKNTLYLQNMS(L/M)RAEDTAVYYCA;
FR4 has a sequence:
(W/R/P/G/C)(G/S/F)(Q/C/P)GT(L/Q)VTV(S/L)(S/E).

In certain embodiments the antigen binding site is one having at least 75%, preferably 80%, more preferably 85%, more preferably 90%, more preferably 95%, more preferably 98% or 99% identity to an antigen binding site shown in Table 3.

In certain embodiments the CDR is one having at least 75%, preferably 80%, more preferably 85%, more preferably 90%, more preferably 95%, more preferably 98% or 99% identity to a CDR shown in Table 1.

Percent sequence identity is determined by conventional methods, by means of computer programs known in the art such as GAP provided in the GCG program package (Program Manual for the Wisconsin Package, Version 8, August 1994, Genetics Computer Group, 575 Science Drive, Madison, Wisconsin, USA 53711) as disclosed in Needleman, S. B. and Wunsch, C. D., (1970), Journal of Molecular Biology, 48, 443-453, which is hereby incorporated by reference in its entirety. GAP is used with the following settings for polypeptide sequence comparison: GAP creation penalty of 3.0 and GAP extension penalty of 0.1.

Sequence identity of polynucleotide molecules is determined by similar methods using GAP with the following settings for DNA sequence comparison: GAP creation penalty of 5.0 and GAP extension penalty of 0.3.

In other embodiments there is provided an antigen binding site or CDR and/or FR having 5 a sequence as described above and including one or more mutations for increasing the affinity of said site for binding to an anti-P2X₇ receptor. The mutation may result in a substitution, insertion or deletion of a residue in one or more of CDR1, CDR2 or CDR3, or one or more or FR1, FR2, FR3 or FR4.

Marks et al. (1992) BioTechnology 10:779, which describes affinity maturation by VH and VL domain shuffling; Barbas et al. (1994) Proc Nat. Acad. Sci. USA 9 1:3809; Schier et al. (1995) Gene 169:147-155; Yelton et al. (1995) J. Immunol. 155:1994; Jackson et al (1995), J. Immunol. 154 (7): 3310; and Hawkins et al, (1992) J. Mol. Biol. 226:889, which describe random mutagenesis of hypervariable region and/or framework residues, are examples of procedures known in the art for affinity maturation of antigen binding sites. In certain embodiments, a nucleic acid encoding one or more of the sequences shown in Table 1 or Table 3 is mutagenized to create a diverse library of sequences. The library is then screened against a target including an epitope of a non functional P2X₇ receptor. An exemplary method is shown in the Examples herein.

In another embodiment there is provided an antigen binding site as described above wherein an amino acid sequence forming one or more of FR1, CDR1, FR2, CDR2, FR3, CDR3 and FR4 is derived from a human sequence or in the form of a human sequence.

The antigen binding site may be presented in a humanized form including non-human (e.g., murine) and human immunoglobulin sequences. Typically all but the CDR sequences of the antigen binding site are from a non-human species such as mouse, rat or rabbit. In some instances, framework residues of the antigen binding site may also be non human. Where the antigen binding site is provided in the form of a whole antibody, typically at least a portion of an immunoglobulin constant region (Fc) is human, thereby allowing various human effector functions.

Methods for humanizing non-human antigen binding sites are well known in the art, examples of suitable processes including those in Jones et al., (1986) Nature, 321:522; Riechmann et al., (1988) Nature, 332:323; Verhoeyen et al., (1988) Science, 239:1534.

Phage display methods described herein using antibody libraries derived from human immunoglobulin sequences are useful for generating human antigen binding sites and human antibodies.

Also, transgenic mammals that are incapable of expressing functional endogenous immunoglobulins, but which can express human immunoglobulin genes can be used. These mice may be generated by random or targeted insertion of the human heavy and light chain immunoglobulin genes into embryonic stem cells. The host heavy and light chain immunoglobulin genes may be rendered non-functional by the insertion or by some other recombination event, for example by homozygous deletion of the host JH region. The transfected embryonic stem cells are expanded and microinjected into blastocysts to produce chimeric mice that are then bred to produce homozygous offspring that express human antigen binding sites. After immunization with a P2X₇ epitope, human monoclonal antibodies can be obtained. One benefit of transgenic animal systems is that it is possible to produce therapeutically useful isotypes because the human immunoglobulin transgenes rearrange during B-cell differentiation and subsequently undergo class switching and somatic mutation in the transgenic mice.

Variable domains including CDRs and FRs of the invention may have been made less immunogenic by replacing surface-exposed residues so as to make the antibody appear as self to the immune system. Padlan, E. A., 1991, Mol. Immunol. 28, 489 provides an exemplary method. Generally, affinity is preserved because the internal packing of amino acid residues in the vicinity of the antigen binding site remains unchanged and generally CDR residues or adjacent residues which influence binding characteristics are not to be substituted in these processes.

In another embodiment there is provided an anti P2X₇ receptor immunoglobulin variable domain, antibody, Fab, dab or scFv including an antigen binding site as described above. In certain embodiments the antigen binding site has a sequence as shown in Table 3.

Lower molecular weight antibody fragments, as compared with whole antibodies may have improved access to solid tumors and more rapid clearance which may be particularly useful in therapeutic and in vivo diagnostic applications.

Various techniques have been developed for the production of antibody fragments including proteolytic digestion of intact antibodies and recombinant expression in host cells. With regard to the latter, as described below, Fab, Fv and scFv antibody fragments can all be expressed in and secreted from E. coli, antibody fragments can be isolated from the antibody phage libraries and Fab′-SH fragments can be directly recovered from E. coli and chemically coupled to form F(ab′)2 fragments. In another approach, F(ab′)2 fragments are isolated directly from recombinant host cell culture.

In certain embodiments, the antigen binding site is provided in the form of a single chain Fv fragment (scFv). Fv and scFv are suitable for reduced nonspecific binding during in vivo use as they have intact combining sites that are devoid of constant regions. Fusion proteins including scFv may be constructed to yield fusion of an effector protein at either the amino or the carboxy terminus of an scFv.

In another embodiment there is provided a diabody or triabody or other multispecific antibody including an antigen binding site as described above. Multispecific antibodies may be assembled using polypeptide domains that allow for multimerization. Examples include the CH2 and CH3 regions of the Fc and the CH1 and Ckappa/lambda regions. Other naturally occurring protein multimerization domains may be used including leucine zipper domain (bZIP), helix-loop-helix motif, Src homology domain (SH2, SH3), an EF hand, a phosphotyrosine binding (PTB) domain, or other domains known in the art.

In another embodiment there is provided a fusion domain or heterologous protein including an antigen binding site, immunoglobulin variable domain, antibody, Fab, dab, scFv, diabody or triabody as described above.

A heterologous polypeptide may be recombinantly fused or chemically conjugated to an N-or C-terminus of an antigen binding site or molecule containing same of the invention.

The heterologous polypeptide to which the antibody or antigen binding site is fused may be useful to target to the P2X₇ receptor expressing cells, or useful to some other function such as purification, or increasing the in vivo half life of the polypeptides, or for use in immunoassays using methods known in the art.

In preferred embodiments, a marker amino acid sequence such as a hexa-histidine peptide is useful for convenient purification of the fusion protein. Others include, but are not limited to, the “HA” tag, which corresponds to an epitope derived from the influenza hemagglutinin protein and the “flag” tag.

Further, the antigen binding site, immunoglobulin variable domain, antibody, Fab, dab, scFv, diabody or triabody of the invention may be modified by glycosylation, acetylation, pegylation, phosphorylation, amidation, derivatization by known protecting/blocking groups, proteolytic cleavage, linkage to a cellular ligand or other protein, etc.

Antigen binding sites of the invention can be composed of amino acids joined to each other by peptide bonds or modified peptide bonds, i.e., peptide isosteres, and may contain amino acids other than the 20 gene-encoded amino acids. Antigen binding sites of the invention may be modified by natural processes, such as posttranslational processing, or by chemical modification techniques which are well known in the art. Such modifications are well described in basic texts, as well as in research literature. Modifications can occur anywhere in the antigen binding site, including the peptide backbone, the amino acid side-chains and the amino or carboxyl termini, or on moieties such as carbohydrates. It will be appreciated that the same type of modification may be present in the same or varying degrees at several sites in a given antigen binding site. Also, a given antigen binding site may contain many types of modifications. An antigen binding site may be branched, for example, as a result of ubiquitination, and they may be cyclic, with or without branching. Cyclic, branched, and branched cyclic antigen binding sites may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination.

In another embodiment there is provided a conjugate in the form of an antigen binding site, immunoglobulin variable domain, antibody, Fab, dab, scFsv, diabody, triabody or fusion protein as described above conjugated to a cytotoxic agent such as a chemo therapeutic agent, a drug, a growth inhibitory agent, a toxin (e.g., an enzymatically active toxin of bacterial, fungal, plant, or animal origin, or fragments thereof), or a label such as a radioactive isotope (i.e., a radio conjugate). In another aspect, the invention further provides methods of using the immunoconjugates. In one aspect, an immunoconjugate comprises any of the above variable domains covalently attached to a cytotoxic agent or a detectable agent.

In another embodiment there is provided an antibody for binding to an antigen binding site of an immunoglobulin variable domain, antibody, Fab, dab, scFv, diabody, triabody, fusion protein or conjugate as described above.

In another embodiment there is provided a nucleic acid encoding an antigen binding site, immunoglobulin variable domain, antibody, Fab, dab, scFv, diabody, triabody, fusion protein or conjugate as described above.

A polynucleotide encoding an CDR or FR according to any one of the general formulae described above, or an antigen binding site comprised of same, may be generated from a nucleic acid from any source, for example by chemical synthesis or isolation from a cDNA or genomic library. For example a cDNA library may be generated from an antibody producing cell such as a B cell, plasma cell or hybridoma cell and the relevant nucleic acid isolated by PCR amplification using oligonucleotides directed to the particular clone of interest. Isolated nucleic acids may then be cloned into vectors using any method known in the art. The relevant nucleotide sequence may then be mutagenized using methods known in the art e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N. Y. and Ausubel et al., eds., 1998, Current Protocols in Molecular Biology, John Wiley & Sons, NY), to generate antigen binding sites having a different amino acid sequence, for example to create amino acid substitutions, deletions, and/or insertions.

In another embodiment there is provided a vector including a nucleic acid described above. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The antigen binding site may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the antigen binding site-encoding DNA that is inserted into the vector. The signal sequence may be a prokaryotic signal sequence selected, for example, from the group of the alkaline phosphatase, penicillinase, Ipp, or heat-stable enterotoxin II leaders. For yeast secretion the signal sequence may be, e.g., the yeast invertase leader, alpha factor leader, or acid phosphatase leader or the C. albicans glucoamylase leader. In mammalian cell expression, mammalian signal sequences may be used to direct secretion of the protein, such as signal sequences from secreted polypeptides of the same or related species, as well as viral secretory leaders.

Polynucleotide sequences encoding polypeptide components of the antigen binding site of the invention can be obtained using standard recombinant techniques as described above. Polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides.

In general, plasmid vectors containing replicon and control sequences which are derived from species compatible with the host cell are used in connection with these hosts. Both expression and cloning vectors contain a nucleic acid sequence that enables the vector to replicate in one or more selected host cells, as well as marking sequences which are capable of providing phenotypic selection in transformed cells. Such sequences are well known for a variety of bacteria, yeast, and viruses. The origin of replication from the plasmid pBR322, which contains genes encoding ampicillin (Amp) and tetracycline (Tet) resistance and thus provides easy means for identifying transformed cells, is suitable for most Gram-negative bacteria, the 2 μm plasmid origin is suitable for yeast, and various viral origins (SV40, polyoma, adenovirus, VSV or BPV) are useful for cloning vectors in mammalian cells. pBR322, its derivatives, or other microbial plasmids or bacteriophage may also contain, or be modified to contain, promoters which can be used by the microbial organism for expression of endogenous proteins.

In addition, phage vectors containing replicon and control sequences that are compatible with the host microorganism can be used as transforming vectors in connection with these hosts. For example, bacteriophage such as λGEM™-11 may be utilized in making a recombinant vector which can be used to transform susceptible host cells such as E. coli LE392.

The expression vector of the invention may comprise two or more promoter-cistron (a cistron being segment of DNA that contains all the information for production of single polypeptide) pairs. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the light or heavy chain by removing the promoter from the source DNA via restriction enzyme digestion and inserting the isolated promoter sequence into the vector of the invention. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters recognized by a variety of potential host cells are well known. Promoters suitable for use with prokaryotic hosts include the PhoA promoter, the β-galactamase and lactose promoter systems, alkaline phosphatase, a tryptophan (trp) promoter system and hybrid promoters such as the tac or the trc promoter. Promoters for use in bacterial systems also will contain a Shine-Dalgarno (S.D.) sequence operably linked to the DNA encoding an antigen binding site of the invention. However, other promoters that are functional in bacteria (such as other known bacterial or phage promoters) are suitable as well. Their nucleotide sequences have been published, thereby enabling a skilled person operably to ligate them to cistrons encoding the target light and heavy chains using linkers or adaptors to supply any required restriction sites.

In one aspect of the invention, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP. In one embodiment of the invention, the signal sequences used in both cistrons of the expression system are STII signal sequences or variants thereof.

In another aspect, the production of the immunoglobulins according to the invention can occur in the cytoplasm of the host cell, and therefore does not require the presence of secretion signal sequences within each cistron. In that regard, immunoglobulin light and heavy chains are expressed, folded and assembled to form functional immunoglobulins within the cytoplasm. Certain host strains (e.g., the E. coli trxB⁻ strains) provide cytoplasm conditions that are favourable for disulfide bond formation, thereby permitting proper folding and assembly of expressed protein subunits.

The present invention provides an expression system in which the quantitative ratio of expressed polypeptide components can be modulated in order to maximize the yield of secreted and properly assembled antigen binding sites of the invention. Such modulation is accomplished at least in part by simultaneously modulating translational strengths for the polypeptide components.

In terms of expression in eukaryotic host cells, the vector components generally include, but are not limited to, one or more of the following: a signal sequence, an origin of replication, one or more marker genes, an enhancer element, a promoter, and a transcription termination sequence.

A vector for use in a eukaryotic host cell may also contain a signal sequence or other polypeptide having a specific cleavage site at the N-terminus of the mature protein or polypeptide of interest. The heterologous signal sequence selected preferably is one that is recognized and processed {i.e., cleaved by a signal peptidase) by the host cell. In mammalian cell expression, mammalian signal sequences as well as viral secretory leaders, for example, the herpes simplex gD signal, are available.

The DNA for such precursor region is ligated in reading frame to DNA encoding the antibody.

Generally, an origin of replication component is not needed for mammalian expression vectors. For example, the SV40 origin may typically be used only because it contains the early promoter.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

An example of suitable selectable markers for mammalian cells are those that enable the identification of cells competent to take up the antigen binding site-encoding nucleic acid, such as DHFR or thymidine kinase, metallothionein-I and-II, preferably primate metallothionein genes, adenosine deaminase, ornithine decarboxylase, etc. An appropriate host cell when wild-type DHFR is employed is the CHO cell line deficient in DHFR activity (e.g., ATCC CRL-9096), prepared and propagated. For example, cells transformed with the DHFR selection gene are first identified by culturing all of the transformants in a culture medium that contains methotrexate (Mtx), a competitive antagonist of DHFR. Alternatively, host cells (particularly wild-type hosts that contain endogenous DHFR) transformed or co-transformed with DNA sequences encoding an antibody, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418.

Expression and cloning vectors usually contain a promoter operably linked to the antigen binding site encoding nucleic acid sequence to direct mRNA synthesis. Promoters recognized by a variety of potential host cells are well known.

Eukaryotic genes generally have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase or other glycolytic enzymes including enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization.

Antigen binding site transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the antigen binding site by higher eukaryotes may be increased by inserting an enhancer sequence into the vector. Enhancer sequences include those known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding an antigen binding site.

In another embodiment there is provided a cell including a vector or nucleic acid described above. The nucleic acid molecule or vector may be present in the genetically modified host cell or host either as an independent molecule outside the genome, preferably as a molecule which is capable of replication, or it may be stably integrated into the genome of the host cell or host.

The host cell of the present invention may be any prokaryotic or eukaryotic cell.

Examples of prokaryotic cells are those generally used for cloning like E. coli or Bacillus subtilis. Furthermore, eukaryotic cells comprise, for example, fungal or animal cells.

Examples for suitable fungal cells are yeast cells, preferably those of the genus Saccharomyces and most preferably those of the species Saccharomyces cerevisiae.

Examples of animal cells are, for instance, insect cells, vertebrate cells, preferably mammalian cells, such as e.g. HEK293, NSO, CHO, MDCK, U2-OS, Hela, NIH3T3, MOLT-4, Jurkat, PC-12, PC-3, IMR, NT2N, Sk-n-sh, CaSki, C33A. These host cells, e.g. CHO-cells, may provide post-translational modifications to the antibody molecules of the invention, including leader peptide removal, folding and assembly of H (heavy) and L (light) chains, glycosylation of the molecule at correct sides and secretion of the functional molecule.

Further suitable cell lines known in the art are obtainable from cell line depositories, like the American Type Culture Collection (ATCC).

In another embodiment there is provided an animal including a cell described above. In certain embodiments, animals and tissues thereof containing a transgene are useful in producing the antigen binding sites of the invention. The introduction of the nucleic acid molecules as transgenes into non-human hosts and their subsequent expression may be employed for the production of the antigen binding sites, for example, the expression of such a transgene in the milk of the transgenic animal provide for means of obtaining the antigen binding sites in quantitative amounts. Useful transgenes in this respect comprise the nucleic acid molecules of the invention, for example, coding sequences for the antigen binding sites described herein, operatively linked to promoter and/or enhancer structures from a mammary gland specific gene, like casein or beta-lactoglobulin. The animal may be non-human mammals, most preferably mice, rats, sheep, calves, dogs, monkeys or apes.

In another embodiment there is provided a pharmaceutical composition including an antigen binding site, immunoglobulin variable domain, antibody, Fab, dab, scFv, diabody, triabody, fusion protein or conjugate as described above and a pharmaceutically acceptable carrier, diluent or excipient.

Methods of preparing and administering antigen binding sites thereof to a subject in need thereof are well known to or are readily determined by those skilled in the art. The route of administration of the antigen binding site may be oral, parenteral, by inhalation or topical.

The term parenteral as used herein includes, e.g., intravenous, intraarterial, intraperitoneal, intramuscular, subcutaneous, rectal or vaginal administration.

While all these forms of administration are clearly contemplated as being within the scope of the invention, a form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc.

Preparations for parenteral administration includes sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions, in such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating an active compound (e.g., antigen binding site) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit. Such articles of manufacture will preferably have labels or package inserts indicating that the associated compositions are useful for treating a subject suffering from, or predisposed disorders.

Effective doses of the compositions of the present invention, for treatment of disorders as described herein vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages may be titrated using routine methods known to those of skill in the art to optimize safety and efficacy.

For treatment of certain disorders with an antigen binding site, the dosage can range, e.g., from about 0.0001 to 100 mg/kg, and more usually 0.01 to 5 mg/kg (e.g., 0.02 mg/kg, 0.25 mg/kg, 0.5 mg/kg, 0.75 mg/kg, lmg/kg, 2 mg/kg, etc.), of the host body weight. For example dosages can be 1 mg/kg body weight or 10 mg/kg body weight or within the range of 1-10 mg/kg, preferably at least 1 mg/kg. Doses intermediate in the above ranges are also intended to be within the scope of the invention. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. Exemplary dosage schedules include 1-10 mg/kg or mg/kg on consecutive days, 30 mg/kg on alternate days or 60 mg/kg weekly. In some methods, two or more antigen binding sites with different binding specificities are administered simultaneously, in which case the dosage of each antigen binding sites administered falls within the ranges indicated.

An antigen binding site disclosed herein can be administered on multiple occasions. Intervals between single dosages can be weekly, monthly or yearly. Intervals can also be irregular as indicated by measuring blood levels of target polypeptide or target molecule in the patient. In some methods, dosage is adjusted to achieve a plasma polypeptide concentration of 1-1000 μg/ml and in some methods 25-300 μg/ml. Alternatively, antigen binding sites can be administered as a sustained release formulation, in which case less frequent administration is required. Dosage and frequency vary depending on the half-life of the antigen binding site in the patient. The half-life of an antigen binding site can also be prolonged via fusion to a stable polypeptide or moiety, e.g., albumin or PEG. In general, humanized antibodies show the longest half-life, followed by chimeric antibodies and nonhuman antibodies. In one embodiment, the antigen binding site of the invention can be administered in unconjugated form. In another embodiment the antigen binding sites for use in the methods disclosed herein can be administered multiple times in conjugated form. In still another embodiment, the antigen binding sites of the invention can be administered in unconjugated form, then in conjugated form, or vice versa.

The dosage and frequency of administration can vary depending on whether the treatment is prophylactic or therapeutic. In prophylactic applications, compositions comprising antibodies or a cocktail thereof are administered to a patient not already in the disease state or in a pre-disease state to enhance the patient's resistance. Such an amount is defined to be a “prophylactic effective dose.” In this use, the precise amounts again depend upon the patient's state of health and general immunity, but generally range from 0.1 to 25 mg per dose, especially 0.5 to 2.5 mg per dose. A relatively low dosage is administered at relatively infrequent intervals over a long period of time. Some patients continue to receive treatment for the rest of their lives.

In therapeutic applications, a relatively high dosage (e.g., from about 1 to 400 mg/kg of binding molecule, e.g., antigen binding site per dose, with dosages of from 5 to 25 mg being more commonly used for radioimmunoconjugates and higher doses for cytotoxin-drug conjugated molecules) at relatively short intervals is sometimes required until progression of the disease is reduced or terminated, and preferably until the patient shows partial or complete amelioration of symptoms of disease. Thereafter, the patent can be administered a prophylactic regime.

In one embodiment, a subject can be treated with a nucleic acid molecule encoding an antigen binding site (e.g., in a vector). Doses for nucleic acids encoding polypeptides range from about 10 ng to 1 g, 100 ng to 100 mg, 1 μg to 10 mg, or 30-300 μg DNA per patient. Doses for infectious viral vectors vary from 10-100, or more, virions per dose.

Therapeutic agents can be administered by parenteral, topical, intravenous, oral, subcutaneous, intraarterial, intracranial, intraperitoneal, intranasal or intramuscular means for prophylactic and/or therapeutic treatment, in some methods, agents are injected directly into a particular tissue where non-functional P2X₇ receptor cells have accumulated, for example intracranial injection. Intramuscular injection or intravenous infusion are preferred for administration of antibody, in some methods, particular therapeutic antibodies are injected directly into the cranium, in some methods, antibodies are administered as a sustained release composition or device.

An antigen binding site of the invention can optionally be administered in combination with other agents that are effective in treating the disorder or condition in need of treatment (e.g., prophylactic or therapeutic).

In another embodiment there is provided a pharmaceutical composition including an antigen binding site, immunoglobulin variable domain, antibody, Fab, dab, scFv, diabody, triabody, fusion protein or conjugate as described above, a diluent and optionally a label.

In certain embodiments, the antigen binding sites or molecule including same are detectably labelled. Many different labels can be used including enzymes, radioisotopes, colloidal metals, fluorescent compounds, chemiluminescent compounds, and bioluminescent compounds. Fluorochromes (fluorescein, rhodamine, Texas Red, etc.), enzymes (horse radish peroxidase, β-galactosidase, alkaline phosphatase etc.), radioactive isotopes (³²P or ¹²⁵I), biotin, digoxygenin, colloidal metals, chemi- or bioluminescent compounds (dioxetanes, luminol or acridiniums) are commonly used.

Detection methods depend on the type of label used and include autoradiography, fluorescence microscopy, direct and indirect enzymatic reactions. Examples include Westernblotting, overlay-assays, RIA (Radioimmuno Assay) and IRMA (Immune Radioimmunometric Assay), EIA (Enzyme Immuno Assay), ELISA (Enzyme Linked Immuno Sorbent Assay), FIA (Fluorescent Immuno Assay), and CLIA (Chemioluminescent Immune Assay).

In another embodiment there is provided a kit or article of manufacture including an antigen binding site, immunoglobulin variable domain, antibody, Fab, dab, scFv, diabody, triabody, fusion protein, conjugate or pharmaceutical composition as described above.

In other embodiments there is provided a kit for use in a therapeutic application mentioned above, the kit including:
a container holding a therapeutic composition in the form of one or more of an antigen binding site, immunoglobulin variable domain, antibody, Fab, dab, scFv, diabody, triabody, fusion protein, conjugate or pharmaceutical composition;
a label or package insert with instructions for use.

In certain embodiments the kit may contain one or more further active principles or ingredients for treatment of a cancer or for preventing a cancer-related complication described above, or a condition or disease associated with non functional P2X₇ receptor expression.

The kit or “article of manufacture” may comprise a container and a label or package insert on or associated with the container. Suitable containers include, for example, bottles, vials, syringes, blister pack, etc. The containers may be formed from a variety of materials such as glass or plastic. The container holds a therapeutic composition which is effective for treating the condition and may have a sterile access port (for example the container may be an intravenous solution bag or a vial having a stopper pierceable by a hypodermic injection needle). The label or package insert indicates that the therapeutic composition is used for treating the condition of choice. In one embodiment, the label or package insert includes instructions for use and indicates that the therapeutic composition can be used to treat a cancer or to prevent a complication stemming from cancer.

The kit may comprise (a) a therapeutic composition; and (b) a second container with a second active principle or ingredient contained therein. The kit in this embodiment of the invention may further comprise a package insert indicating that the and other active principle can be used to treat a disorder or prevent a complication stemming from cancer. Alternatively, or additionally, the kit may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In certain embodiments the therapeutic composition may be provided in the form of a device, disposable or reusable, including a receptacle for holding the therapeutic composition. In one embodiment, the device is a syringe. The device may hold 1-2 mL of the therapeutic composition. The therapeutic composition may be provided in the device in a state that is ready for use or in a state requiring mixing or addition of further components.

In another embodiment there is provided a kit or article of manufacture including an antigen binding site, immunoglobulin variable domain, antibody, Fab, dab, scFv, diabody, triabody, fusion protein, conjugate or a diagnostic composition as described above.

In other embodiments there is provided a kit for use in a diagnostic application mentioned above, the kit including:
a container holding a diagnostic composition in the form of one or more of an antigen binding site, immunoglobulin variable domain, antibody, Fab, dab, scFv, diabody, triabody, fusion protein or conjugate;
a label or package insert with instructions for use.

The kit or “article of manufacture” may comprise a container and a label or package insert on or associated with the container. Suitable containers include, for example, bottles, vials, syringes, blister pack, etc. The containers may be formed from a variety of materials such as glass or plastic. The container holds a diagnostic composition which is effective for detection of cancer and may have a sterile access port (for example the container may be an intravenous solution bag or a vial having a stopper pierceable by a hypodermic injection needle). The label or package insert indicates that the diagnostic composition is used for detecting the condition of choice. In one embodiment, the label or package insert includes instructions for use and indicates that the diagnostic composition can be used to detect a cancer or a disease or condition characterised by non functional P2X₇ receptor expression.

The kit may comprise (a) a diagnostic composition; and (b) a second container with a second diagnostic agent or second label contained therein. It may further include other materials desirable from a commercial and user standpoint, including other buffers, diluents, filters etc.

In another embodiment there is provided a method for producing an anti P2X₇ antigen binding site as described above including expressing a nucleic acid as described above in a cell or non human animal as described above.

The production of an antigen binding site of the invention generally requires an expression vector containing a polynucleotide that encodes the antigen binding site of the invention. A polynucleotide encoding an antigen binding site of the invention may be obtained and sub cloned into a vector for the production of an antigen binding site by recombinant DNA technology using techniques well-known in the art, including techniques described herein.

Many different expression systems are contemplated including the use of mammalian cells including human cells for production and secretion of antigen binding sites. Examples of cells include 293F, CHO and the NSO cell line.

Expression vectors containing protein coding sequences and appropriate transcriptional and translational control signals can be constructed using methods known in the art. These include in vitro recombinant DNA techniques, synthetic techniques and in vivo genetic recombination. In certain embodiments there is provided a replicable vector having a nucleic acid encoding an antigen binding site operably linked to a promoter.

Cells transfected with an expression vector may be cultured by conventional techniques to produce an antigen binding site. Thus, in certain embodiments, there is provided host cells or cell transfectants containing a polynucleotide encoding an antigen binding site of the invention operably linked to a promoter. The promoter may be heterologous. A variety of host-expression vector systems may be utilized and in certain systems the transcription machinery of the vector system is particularly matched to the host cell. For example, mammalian cells such as Chinese hamster ovary cells (CHO) may be transfected with a vector including the major intermediate early gene promoter element from human cytomegalovirus. Additionally or alternatively, a host cell may be used that modulates the expression of inserted sequences, or modifies and processes the gene product as required, including various forms of post translational modification. Examples of mammalian host cells having particular post translation modification processes include CHO, VERY, BHK, Hela, COS, MDCK, 293, 3T3, W138, BT483, Hs578T, HTB2, BT2O and T47D, NSO, CRL7O3O and HsS78Bst cells.

Depending upon the use intended for the protein molecule, a number of bacterial expression vectors may be advantageously selected. In one example, vectors that cause the expression of high levels of fusion protein products that are readily purified, such as the E. coli expression vector pUR278 may be used where a large quantity of an antigen binding site is to be produced. The expression product may be produced in the form of a fusion protein with lacZ. Other bacterial vectors include pIN vectors and the like. pGEX vectors may also be used to express foreign polypeptides as fusion proteins with glutathione-S-transferase (GST). These fusion proteins are generally soluble and can easily be purified from lysed cells by adsorption and binding to glutathione-agarose affinity matrix followed by elution in the presence of free glutathione. A thrombin and/or factor Xa protease cleavage site may be provided in the expressed polypeptide so that the cloned target gene product can be released from the GST moiety.

Autographa californica nuclear polyhedrosis virus (AcNPV) may be used as a vector to express foreign genes in an insect system including Spodoptera frugiperda cells. The particular promoter used may depend on where the protein coding is inserted into the sequence. For example, the sequence may be cloned individually into the polyhedrin gene and placed under control of the polyhedrin promoter.

Virus based expression systems may be utilized with mammalian cells such as an adenovirus whereby the coding sequence of interest may be ligated to the adenoviral late promoter and tripartite leader sequence. In vitro or in vivo recombination may then be used to insert this chimeric gene into the adenoviral genome. Insertions into region E1 or E3 will result in a viable recombinant virus that is capable of expressing the antigen binding site in infected host cells. Specific initiation signals including the ATG initiation codon and adjacent sequences may be required for efficient translation of inserted antigen binding site coding sequences. Initiation and translational control signals and codons can be obtained from a variety of origins, both natural and synthetic. Transcription enhancer elements and transcription terminators may be used to enhance the efficiency of expression of a viral based system.

Where long-term, high-yield production of recombinant proteins is required, stable expression is preferred. Generally a selectable marker gene is used whereby following transfection, cells are grown for 1-2 days in an enriched media and then transferred to a medium containing a selective medium in which cells containing the corresponding selectable marker, for example, antibiotic resistance can be screened. The result is that cells that have stably integrated the plasmid into their chromosomes grow and form foci that in turn can be cloned and expanded into cell lines. The herpes simplex virus thymidine kinase, hypoxanthineguanine phosphoribosyltransferase and adenine phosphoribosyltransferase genes are examples of genes that can be employed in tk⁻, hgprt⁻ or aprT⁻ cells, respectively thereby providing appropriate selection systems. The following genes: dhfr, which confers resistance to methotrexate; gpt, which confers resistance to mycophenolic acid; neo, which confers resistance to the aminoglycoside G-418; and hygro, which confers resistance to hygromycin are examples of genes that can be used in anti metabolite selection systems.

An antigen binding site of the invention may be purified by a recombinant expression system by known methods including ion exchange chromatography, affinity chromatography (especially affinity for the specific antigens Protein A or Protein G) and gel filtration column chromatography), centrifugation, differential solubility, or by any other standard technique for the purification of proteins. Purification may be facilitated by providing the antigen binding site in the form of a fusion protein.

Large quantities of the antigen binding sites of the invention may be produced by a scalable process starting with a pilot expression system in a research laboratory that is scaled up to an analytical scale bioreactor (typically from 5 L to about 50 L bioreactors) or production scale bioreactors (for example, but not limited to 75 L, 100 L, 150 L, 300 L, or 500 L). Desirable scalable processes include those wherein there are low to undetectable levels of aggregation as measured by HPSEC or rCGE, typically no more than 5% aggregation by weight of protein down to no more than 0.5% by weight aggregation of protein. Additionally or alternatively, undetectable levels of fragmentation measured in terms of the total peak area representing the intact antigen binding site may be desired in a scalable process so that at least 80% and as much as 99.5% or higher of the total peak area represents intact antigen binding site. In other embodiments, the scalable process of the invention produces antigen binding sites at production efficiency of about from 10 mg/L to about 300 mg/L or higher.

In another embodiment there is provided a method for the treatment of a disease or condition characterised by non functional P2X₇ receptor expression in an individual including the step of providing an antigen binding site, immunoglobulin variable domain, antibody, Fab, dab, scFv, diabody, triabody, fusion protein, conjugate or pharmaceutical composition as described above to an individual requiring treatment for said condition. Typically the condition is cancer, especially an epithelial cancer as described herein.

Pre-neoplastic and neoplastic diseases are particular examples to which the methods of the invention may be applied. Broad examples include breast tumors, colorectal tumors, adenocarcinomas, mesothelioma, bladder tumors, prostate tumors, germ cell tumor, hepatoma/cholongio, carcinoma, neuroendocrine tumors, pituitary neoplasm, small 20 round cell tumor, squamous cell cancer, melanoma, atypical fibroxanthoma, seminomas, nonseminomas, stromal leydig cell tumors, Sertoli cell tumors, skin tumors, kidney tumors, testicular tumors, brain tumors, ovarian tumors, stomach tumors, oral tumors, bladder tumors, bone tumors, cervical tumors, esophageal tumors, laryngeal tumors, liver tumors, lung tumors, vaginal tumors and Wilm's tumor.

Examples of particular cancers include but are not limited to adenocarcinoma, adenoma, adenofibroma, adenolymphoma, adontoma, AIDS related. cancers, acoustic neuroma, acute lymphocytic leukemia, acute myeloid leukemia, adenocystic carcinoma, adrenocortical cancer, agnogenic myeloid metaplasia, alopecia, alveolar soft-part sarcoma, ameloblastoma, angiokeratoma, angiolymphoid hyperplasia with eosinophilia, angioma sclerosing, angiomatosis, apudoma, anal cancer, angiosarcoma, aplastic anaemia, astrocytoma, ataxia-telangiectasia, basal cell carcinoma (skin), bladder cancer, bone cancers, bowel cancer, brain stem glioma, brain and CNS tumors, breast cancer, branchioma, CNS tumors, carcinoid tumors, cervical cancer, childhood brain tumors, childhood cancer, childhood leukemia, childhood soft tissue sarcoma, chondrosarcoma, choriocarcinoma, chronic lymphocytic leukemia, chronic myeloid leukemia, colorectal cancers, cutaneous T-cell lymphoma, carcinoma (e.g. Walker, basal cell, basosquamous, Brown-Pearce, ductal, Ehrlich tumor, Krebs 2, Merkel cell, mucinous, non-small cell lung, oat cell, papillary, scirrhous, bronchiolar, bronchogenic, squamous cell, and transitional cell), carcinosarcoma, cervical dysplasia, cystosarcoma phyllodies, cementoma, chordoma, choristoma, chondrosarcoma, chondroblastoma, craniopharyngioma, cholangioma, cholesteatoma, cylindroma, cystadenocarcinoma, cystadenoma, dermatofibrosarcoma-protuberans, desmoplastic-small-round-cell-tumor, ductal carcinoma, dysgerminoam, endocrine cancers, endometrial cancer, ependymoma, esophageal cancer, Ewing's sarcoma, extra-hepatic bile duct cancer, eye cancer, eye: melanoma, retinoblastoma, fallopian tube cancer, fanconi anaemia, fibroma, fibrosarcoma, gall bladder cancer, gastric cancer, gastrointestinal cancers, gastrointestinal-carcinoid-tumor, genitourinary cancers, germ cell tumors, gestationaltrophoblastic-disease, glioma, gynaecological cancers, giant cell tumors, ganglioneuroma, glioma, glomangioma, granulosa cell tumor, gynandroblastoma, haematological malignancies, hairy cell leukemia, head and neck cancer, hepatocellular cancer, hereditary breast cancer, histiocytosis, Hodgkin's disease, human papillomavirus, hydatidiform mole, hypopharynx hypercalcemia, cancer, hamartoma, hemangioendothelioma, hemangioma, hemangiopericytoma, hemangiosarcoma, hemangiosarcoma, histiocytic disorders, histiocytosis malignant, histiocytoma, hepatoma, hidradenoma, hondrosarcoma, immunoproliferative small, opoma, ontraocular melanoma, islet cell cancer, Kaposi's sarcoma, kidney cancer, langerhan's cell-histiocytosis, laryngeal cancer, leiomyosarcoma, leukemia, li-fraumeni syndrome, lip cancer, liposarcoma, liver cancer, lung cancer, lymphedema, lymphoma, Hodgkin's lymphoma, non-Hodgkin's lymphoma, leigomyosarcoma, leukemia (e.g. b-cell, mixed cell, null-cell, t-cell, t-cell chronic, htlv-ii-associated, lymphangiosarcoma, lymphocytic acute, lymphocytic chronic, mast-cell and myeloid), leukosarcoma, leydig cell tumor, liposarcoma, leiomyoma, leiomyosarcoma, lymphangioma, lymphangiocytoma, lymphagioma, lymphagiomyoma, lymphangiosarcoma, male breast cancer, malignant-rhabdoid-tumor-of-kidney, medulloblastoma, melanoma, Merkel cell cancer, mesothelioma, metastatic cancer, mouth cancer, multiple endocrine neoplasia, mycosis fungoides, myelodysplastic syndromes, myeloma, myeloproliferative disorders, malignant carcinoid syndrome carcinoid heart disease, medulloblastoma, meningioma, melanoma, mesenchymoma, mesonephroma, mesothelioma, myoblastoma, myoma, myosarcoma, myxoma, myxosarcoma, nasal cancer, nasopharyngeal cancer, nephroblastoma, neuroblastoma, neurofibromatosis, Nijmegen breakage syndrome, non-melanoma skin cancer, non-small-cell-lung-cancer-(nsclc), neurilemmoma, neuroblastoma, neuroepithelioma, neurofibromatosis, neurofibroma, neuroma, neoplasms (e.g. bone, breast, digestive system, colorectal, liver), ocular cancers, oesophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal-tumors, pituitary cancer, polycythemia vera, prostate cancer, osteoma, osteosarcoma, ovarian carcinoma, papilloma, paraganglioma, paraganglioma nonchromaffin, pinealoma, plasmacytoma, protooncogene, rare-cancers-and-associated-disorders, renal cell carcinoma, retinoblastoma, rhabdomyosarcoma, Rothmund-Thomson syndrome, reticuloendotheliosis, rhabdomyoma, salivary gland cancer, sarcoma, schwannoma, Sezary syndrome, skin cancer, small cell lung cancer (sclc), small intestine cancer, soft tissue sarcoma, spinal cord tumors, squamous-cell-carcinoma-(skin), stomach cancer, synovial sarcoma, sarcoma (e.g. Ewing's experimental, Kaposi's and mast-cell sarcomas), Sertoli cell tumor, synovioma, testicular cancer, thymus cancer, thyroid cancer, transitional-cell-cancer-(bladder), transitional-cell-cancer-(renal-pelvis-/-ureter), trophoblastic cancer, teratoma, theca cell tumor, thymoma, trophoblastic tumor, urethral cancer, urinary system cancer, uroplakins, uterine sarcoma, uterus cancer, vaginal cancer, vulva cancer, Waldenstrom's-macroglobulinemia and Wilms' tumor.

Other diseases and conditions include various inflammatory conditions. Examples may include a proliferative component. Particular examples include acne, angina, arthritis, aspiration pneumonia, disease, empyema, gastroenteritis, inflammation, intestinal flu, nee, necrotizing enterocolitis, pelvic inflammatory disease, pharyngitis, pid, pleurisy, raw throat, redness, rubor, sore throat, stomach flu and urinary tract infections, chronic inflammatory demyelinating polyneuropathy, chronic inflammatory demyelinating polyradiculoneuropathy, chronic inflammatory demyelinating polyneuropathy or chronic inflammatory demyelinating polyradiculoneuropathy.

In another embodiment there is provided a use of an antigen binding site, immunoglobulin variable domain, antibody, Fab, dab, scFsv, diabody, triabody, fusion protein, conjugate or pharmaceutical composition as described above in the manufacture of a medicament for the treatment of cancer.

Dosage amount, dosage frequency, routes of administration etc are described in detail above.

In another embodiment there is provided a method for the diagnosis of cancer including the step of contacting tissues or cells for which the presence or absence of cancer is to be determined with a reagent in the form of an antigen binding site, immunoglobulin variable domain, antibody, Fab, dab, scFv, diabody, triabody, fusion protein, conjugate or diagnostic composition as described above and detecting for the binding of the reagent with the tissues or cells. The method may be operated in vivo or in vitro.

For in situ diagnosis, the antigen binding site may be administered to the organism to be diagnosed by intravenous, intranasal, intraperitoneal, intracerebral, intraarterial injection or other routes such that a specific binding between an antigen binding site according to the invention with an eptitopic region on the non-functional P2X₇ receptor may occur. The antibody/antigen complex may conveniently be detected through a label attached to the antigen binding site or a functional fragment thereof or any other art-known method of detection.

The immunoassays used in diagnostic applications according to the invention and as described herein typically rely on labelled antigens, antibodies, or secondary reagents for detection. These proteins or reagents can be labelled with compounds generally known to those of ordinary skill in the art including enzymes, radioisotopes, and fluorescent, luminescent and chromogenic substances including, but not limited to coloured particles, such as colloidal gold and latex beads. Of these, radioactive labelling can be used for almost all types of assays and with most variations. Enzyme-conjugated labels are particularly useful when radioactivity must be avoided or when quick results are needed. Fluorochromes, although requiring expensive equipment for their use, provide a very sensitive method of detection. Antibodies useful in these assays include monoclonal antibodies, polyclonal antibodies, and affinity purified polyclonal antibodies.

Alternatively, the antigen binding site may be labelled indirectly by reaction with labelled substances that have an affinity for immunoglobulin, such as protein A or G or second antibodies. The antigen binding site may be conjugated with a second substance and detected with a labelled third substance having an affinity for the second substance conjugated to the antigen binding site. For example, the antigen binding site may be conjugated to biotin and the antigen binding site-biotin conjugate detected using labelled avidin or streptavidin. Similarly, the antigen binding site may be conjugated to a hapten and the antigen binding site-hapten conjugate detected using labelled anti-hapten antibody.

In certain embodiments, immunoassays utilize a double antibody method for detecting the presence of an analyte, wherein, the antigen binding site is labelled indirectly by reactivity with a second antibody that has been labelled with a detectable label. The second antibody is preferably one that binds to antibodies of the animal from which the antigen binding site is derived. In other words, if the antigen binding site is a mouse antibody, then the labelled, second antibody is an anti-mouse antibody. For the antigen binding site to be used in the assay described herein, this label is preferably an antibody-coated bead, particularly a magnetic bead. For the antigen binding site to be employed in the immunoassay described herein, the label is preferably a detectable molecule such as a radioactive, fluorescent or an electrochemiluminescent substance.

An alternative double antibody system, often referred to as fast format systems because they are adapted to rapid determinations of the presence of an analyte, may also be employed within the scope of the present invention. The system requires high affinity between the antigen binding site and the analyte. According to one embodiment of the present invention, the presence of the non-functional P2X₇ receptor is determined using a pair of antigen binding sites, each specific for P2X₇ receptor protein. One of said pairs of antigen binding sites is referred to herein as a “detector antigen binding site” and the other of said pair of antigen binding sites is referred to herein as a “capture antigen binding site”. The antigen binding site of the present invention can be used as either a capture antigen binding site or a detector antigen binding site. The antigen binding site of the present invention can also be used as both capture and detector antigen binding site, together in a single assay. One embodiment of the present invention thus uses the double antigen binding site sandwich method for detecting non-functional P2X₇ receptor in a sample of biological fluid. In this method, the analyte (non-functional P2X₇ receptor protein) is sandwiched between the detector antigen binding site and the capture antigen binding site, the capture antigen binding site being irreversibly immobilized onto a solid support. The detector antigen binding site would contain a detectable label, in order to identify the presence of the antigen binding site-analyte sandwich and thus the presence of the analyte.

Exemplary solid phase substances include, but are not limited to, microtiter plates, test tubes of polystyrene, magnetic, plastic or glass beads and slides which are well known in the field of radioimmunoassay and enzyme immunoassay. Methods for coupling antigen binding sites to solid phases are also well known to those of ordinary skill in the art. More recently, a number of porous material such as nylon, nitrocellulose, cellulose acetate, glass fibers and other porous polymers have been employed as solid supports.

The examples that follow are intended to illustrate but in no way limit the present invention.

### EXAMPLES

### Example 1

### Identifying dAB Leads for Binding to Non Functional Receptors on Live Cells

Objective: The experiments described here have been to find antigen binding sites that bind the E200 peptide.

Background: Antisera that bind P2X₇ have low affinity for P2X₇ as expressed on live cancer cells since the conformation of the epitope target on live cancer cells differs. To identify dAb leads for high affinity binders, we first needed to identify a suitable target, knowing that good sequence diversity of binders is required in order to widen the screening of conformational space to encompass suitable lead compounds. We selected the E200 peptide as a suitable target to identify dAb leads.

Materials and methods: The E200 peptide was made by solid phase synthesis at Chiron Mimotopes. A range of conjugates were synthesized to identify those most likely to be useful for screening purposes. These included protein conjugates BSA, DT and ovalbumin linked to the C-terminal Cys reside on E200 peptide via MCS. A fourth variant involved biotinylating the E200 peptide at the C-terminus.

Suitable lead clones were initially identified as ELISA positives in both solid phase and solution phase screens. These were made against both the unconjugated and the conjugated peptides. Additional peptides were synthesized (200-208 and 207-215) in order to differentiate more completely the binding regions of the various lead clones. Solution properties using SEC-MALLS of the lead clones were tested to ensure they were suitable for further development.

### Results

A large number of first generation leads were identified and isolated that initially bound to the E200 peptide with binding affinity in the uM K_{D} range as measured by Biacore and then bound detectably by flow cytometry to live cancer cells expressing the non-functional P2X₇ receptor target on their surface. Single domain antibodies produced from Domantis phage display library screened against the peptide antigen E200 exhibited a K_{D} of the order of 1 uM using Biacore binding analyses. Lead clones taken forward showed diversity in their binding characteristics. Three lead dAbs, PEP2-2, PEP2-4 and PEP2-5 exhibited the highest affinity when tested on live PC3 human prostate cancer cells by flow cytometry. Additional screening involved the use of standard immunohistochemistry in which normal human and cancer tissue was incubated with the chosen dAb labelled with Myc tag to which a labelled anti-Myc antibody with HRP was added. Diaminobenzoate (DAB) was added to react with any HRP remaining after due washing steps were completed. PEP2-4 and PEP2-5 bound moderately to the tumour tissue but not to normal tissue such as human prostate and skin while PEP2-2 was an example of a dAb lead that showed little effective binding to tissue in the initial screening.

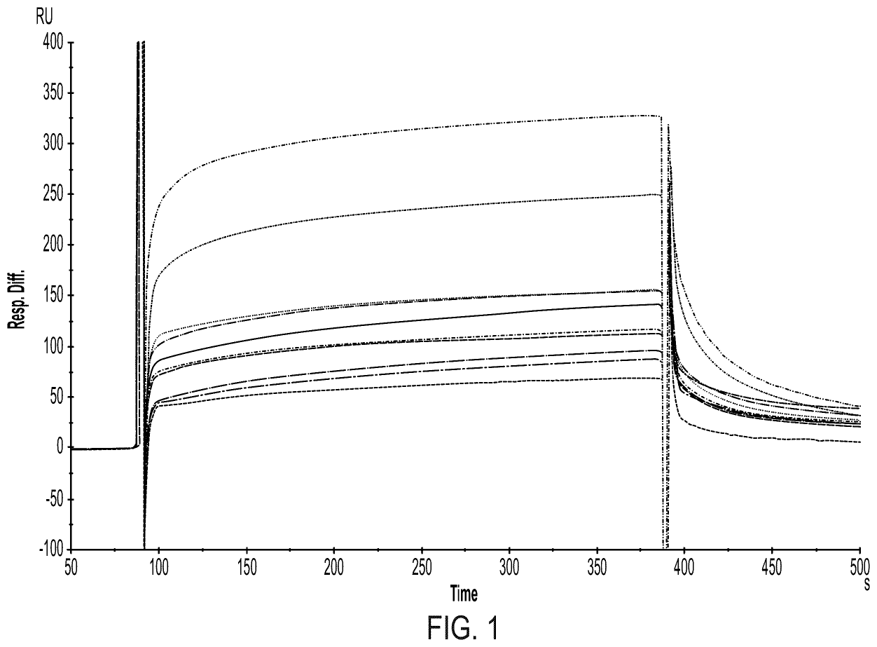

Passive selection was performed using the E200, the E200-BSA conjugate, the E200, ovalbumin conjugate and the E200-DT conjugate peptides while solution screening used the biotinylated peptide then assayed using strepatavidin. Both passive and solution selections of the numerous lead dabs worked well with specific binders demonstrating good sequence diversity in the form of the single V_{H} domains. Screening against the E200 peptide and smaller parts (200-208 and 207-215) revealed the lead dabs bound to different regions. Those with the best solution properties, being the highest monomer solubility were carried forward. Those demonstrating biphasic Biacore binding characteristics were not carried forward. All showed uM binding to the E200 peptide. Ultimately a total of five screening rounds were undertaken as shown in Example 5. An example of the results in Round 2 are shown in FIG. 1.

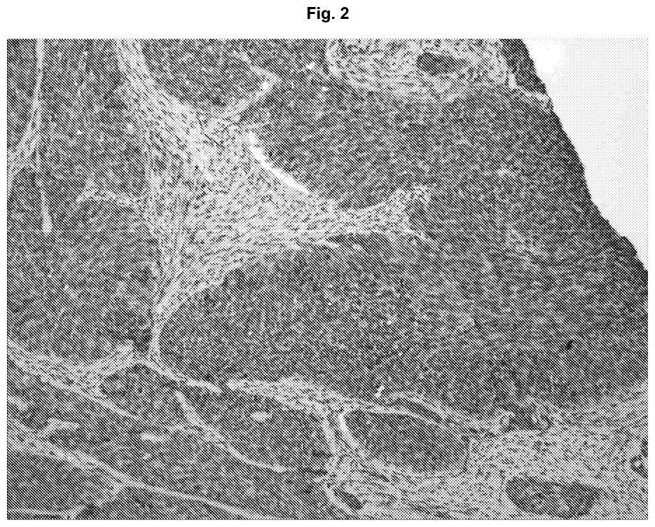

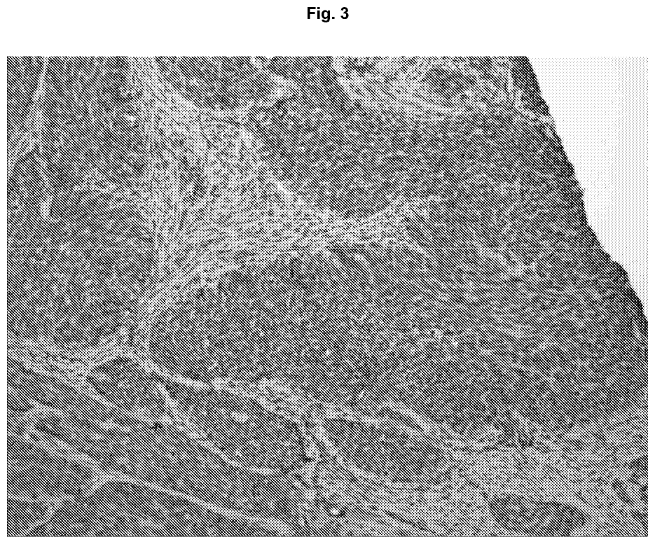

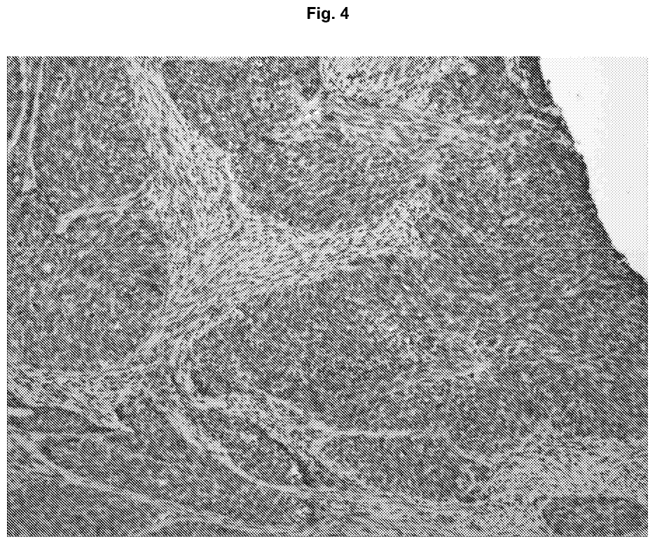

An example of dAb binding to cancer tissue follows in which human cervical cancer tissue was stained with c-Myc-labelled dAb PEP2-4 and then developed using mouse anti-Myc antibody (1:600) followed by the Biocare Medical Mach4 secondary polymer detection system and DAB. To inhibit binding, the peptide substrate was added to the primary at concentrations of 0 (FIG. 2), 25 nM (no loss of binding), 0.25 uM (no loss of binding), 10 uM (no loss of binding), 0.1 mM (FIG. 3) and 1 mM (FIG. 4).

No inhibition of binding was observed at a concentration less than 0.01 mM indicating the ideal for 50% inhibition is about 40-50 uM.

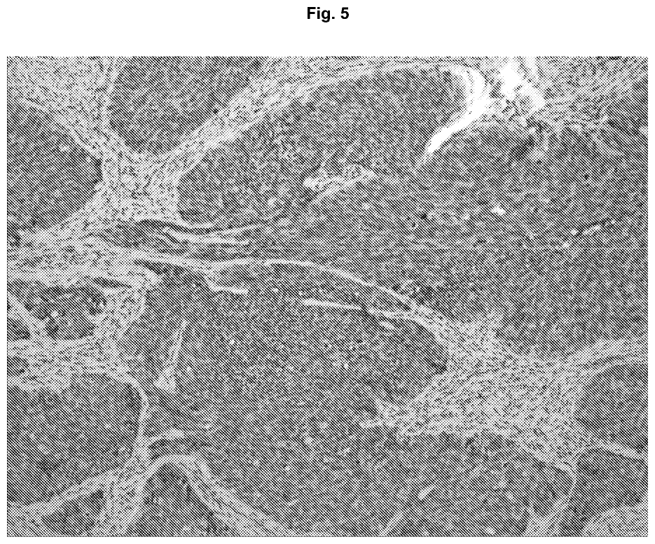

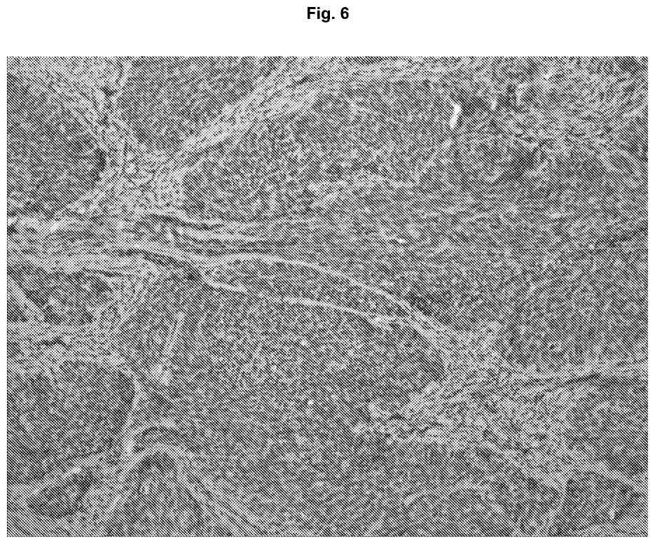

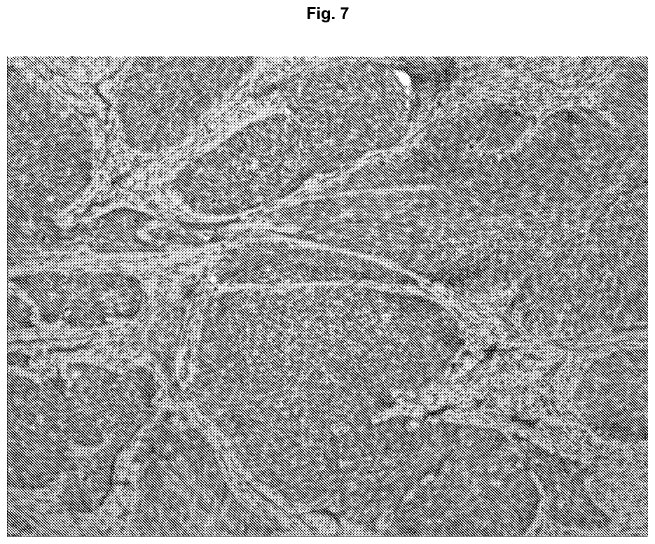

A second set of serial sections is shown in FIGS. 5-7 from different tissue sections, magnification also 10×.

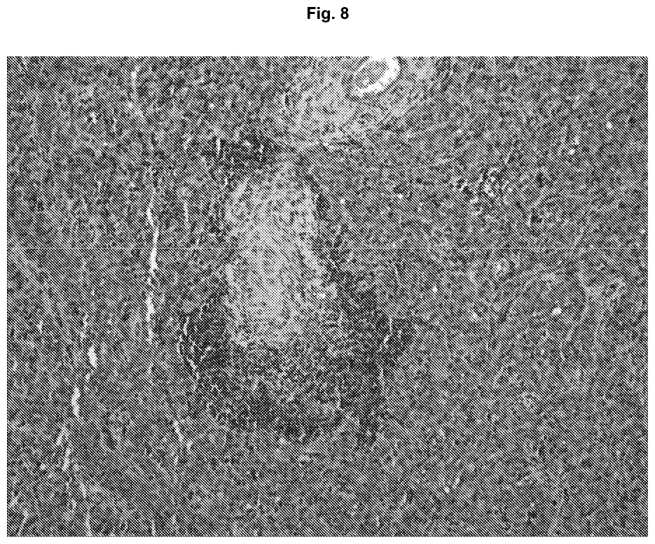

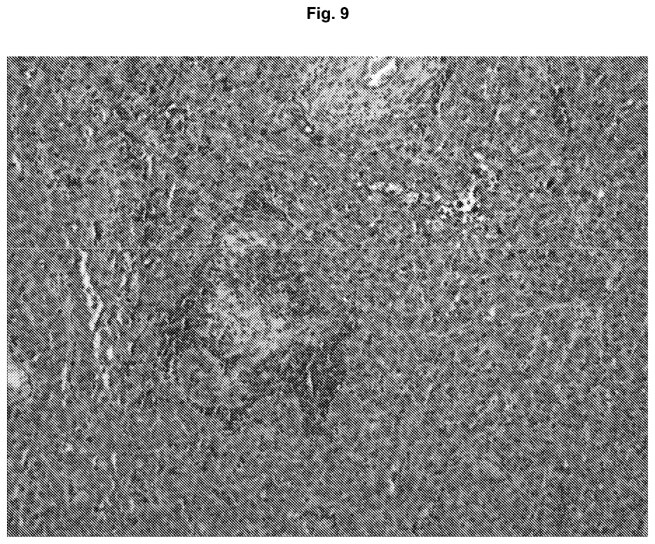

The difference between 0 and 10 uM added competing peptide in contrast was minimal as shown in FIG. 8 (no peptide) and 9 (10 uM peptide).

There is clear inhibition at 100 uM with no inhibition at 10 uM indicating that the binding at 50% inhibition appears to be about 40-50 uM in this system.

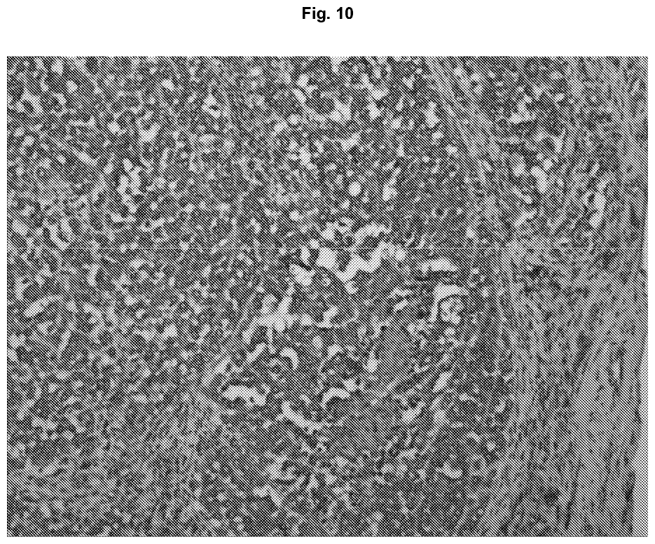

A section of human melanoma tissue similarly stained with 20 nM PEP2-4 dAb is shown in FIG. 10 below:

Conclusion: Antigen binding sites in the form of dAb leads for high affinity P2X₇ binding to PC3 cells were identified. Whether these antigen binding sites interact with a linear or conformational epitope was unknown and subsequently investigated. Refinement of the leads required added screening against a conformational epitope representing the shape of the E200 target antigen binding site as expressed on cancer cells

### Example 2

### Determining Activity of dAb Leads in dAb-Fc Format

Objective: The experiments described here have been to improve affinity of antigen binding sites that bind the E200 peptide through formatting lead dabs as dAb-Fc.

Background: Co-operative binding of the lead dabs was achieved by producing standard format dAb-Fc with human type IgG1 Fc subtype. These formats enabled more considered screening of the lead dAb clones by enabling the elimination of high affinity lead dabs for which formatting as dAb-Fc provided little benefit due to solubility issues. Favourable conformational solutions would then be selected for additional rounds of screening.

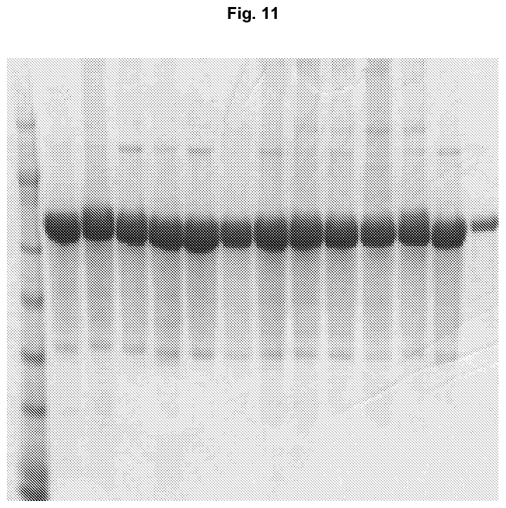

Results: The first formatted dabs PEP2-4 and PEP2-5 that had been chosen as high affinity leads from Example 1 showed little additional binding to the E200 peptide whereas PEP2-2 and others (2-47, 2-42) benefited with a typical improvement in K_{D} of 100-1000 times. Formatting of the various leads resulted in good expression as revealed in the SDS-PAGE gel in FIG. 11.

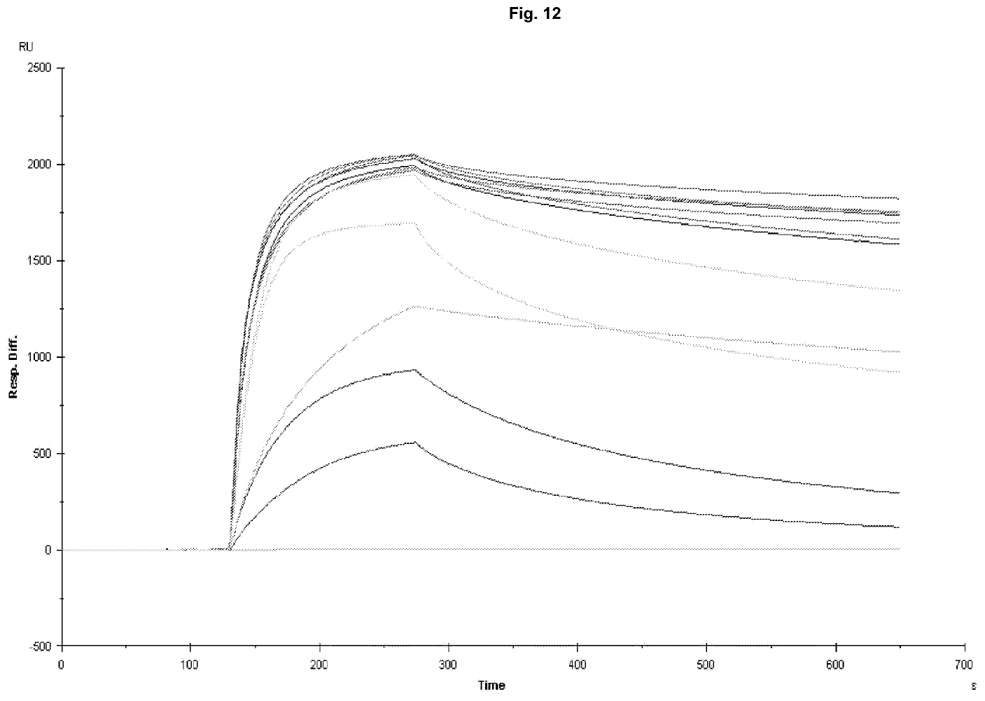

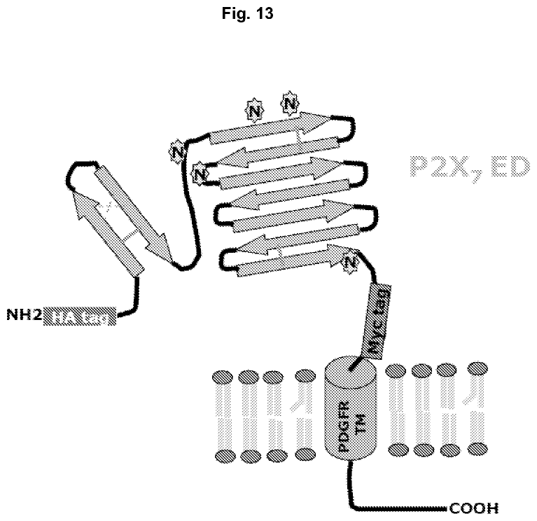

The improvement in binding is evident with the leads including PEP2-2, PEP2-42, PEP2-47 shown in FIG. 12 in which the Biacore chip was coated with 100RU of E200 and each dAb-Fc run at 100 nM.

### Example 3

### Determination of a Conformational Epitope for Screening dAb Leads Against

Objective: The experiments described here have been to determine an appropriate conformational epitope for finding dAbs that bind the E200 peptide and also bind a conformational epitope.

Background: The high affinity binders are to bind to a non functional P2X₇ receptor extra cellular domain. The sequence of P2X₇ is shown in SEQ ID NO: 1. There are a number of possible constructs that could be developed but we had to determine which of these would model the conformational epitopes as observed on a live cancer cell. We particularly needed a target that could be bound to a solid phase for later affinity maturation experiments.

We started with ECD1 that has the structure 47-332 because this constitutes all the amino acids forming the extracellular domain between the transmembrane domains TM1 and TM2 including the putative intramembranous segment at the C-terminus of the segment from 325-332. By including all the residues it was considered likely that the structure around the target E200 would be conserved.

Materials and methods: ECD1 was constructed recombinantly using standard molecular biology procedures and expressed in E. coli cells as soluble protein and formatted as ECD-Fc and in pDisplay for immunofluorescence, Western Blotting and flow cytometry. The pDisplay structure had the form shown in the schematic in FIG. 13.

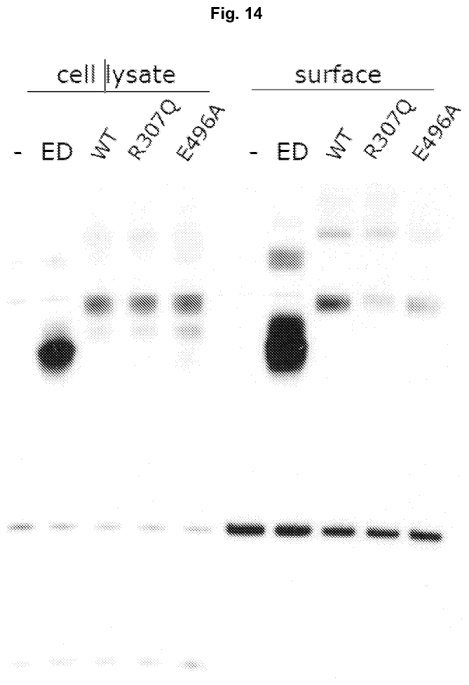

Results: Cell surface expression of P2X₇ in the form of the wild type (WT) and in two non-functional full length mutant forms (R307Q and E496A) were compared along with the ECD1 in HEK293E cells and measured with Western Blot. Cell lysates and cell surface expression was compared in all three forms and the labelling to the ECD1 added. Anti-cadherin was used as a standardisation control. The cells were biotinylated with sulfo-NHS-SS-biotin, the reaction quenched and lysis performed with mild detergent. At this stage an aliquot was retained for indication of total cell protein. Biotinylated protein was captured with neutravidin resin that was washed and eluted with 50 mM DTT. The supernatant was retained for an indication of the intracellular pool of specific protein. The samples were then run on standard reducing SDS PAGE/Westerns (FIG. 14).

Cell surface expression indicates a reduction in the levels of the non-functional mutants compared with WT on the cell surface. The ECD1 expression from expressed pDisplay is efficiently high. This form of the protein is labelled by antibodies to the non-functional form of the receptor, the tumour specific form and can therefore be considered a possible tumour representative form. Monocytes, in contrast, expressing the WT form, were unable to bind the dAbs. The efficiency of binding of the dabs to the pDisplayECD1 was lower than the levels of expression indicated should have been the case. This indicates that the target epitope is sterically hindered from binding on live cells and that the structure of ECD1 is sub-optimal.

Conclusion: While ECD1 construct was bound by dAb leads indicating binding to a conformational epitope, binding was suboptimal which raised the questions concerning whether this construct would be useful for affinity maturation studies.

### Example 4

### Determining a Further Construct for Affinity Maturation of Lead dAbs

Objective: To produce a construct that could be used in affinity maturation studies.

Background: Example 3 revealed that certain ECD isoforms might not reproduce conformational epitopes of P2X₇ as observed on live tumours. We decided to pursue a further construct in the form of the structure 47-306 (ECD2).

Materials and methods: ECD2 was constructed recombinantly as in Example 3, in soluble form, Fc format and as pDisplay for immunofluorescence, Western Blotting and flow cytometry.

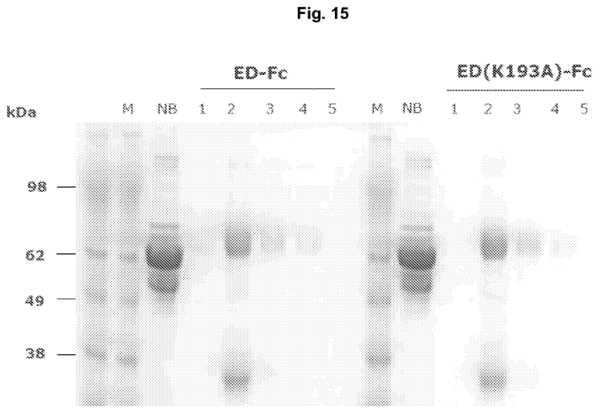

Results: ECD2 expression as an Fc construct is shown in FIG. 15. A reducing SDS-PAGE with Protein A fractions shown in two forms: WT (functional) and K193A (non functional) mutant forms. dAbs were identified that bind the ECD2 construct. NB is an aliquot of the supernatant representing protein not bound by Protein A.

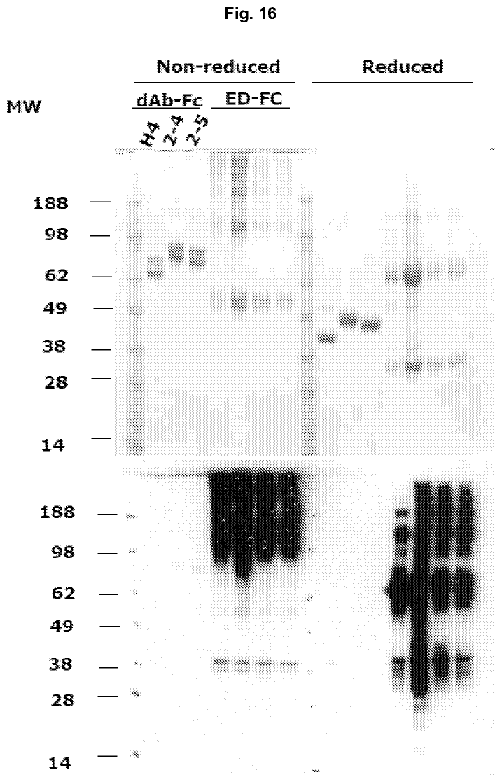

The dAb-Fc species PEP2-4 and PEP2-5 along with control dAb HEL4 were run on non-reduced and reduced gels and corresponding Westerns run on the fractions revealed with anti-P2X₇ antibody (FIG. 16). Both dAb-Fc expression and ECD2-Fc expression is clear. The reduced gels show specific label on the ECD2Fc of the anti-P2X₇ antibody at 62 kDa with a lower molecular weight proteolytic fragment (single chain) at 31 kDa. The corresponding Western shows reactivity with both ECD2 bands but none with HEL4Fc, PEP2-4Fc or PEP2-5Fc.

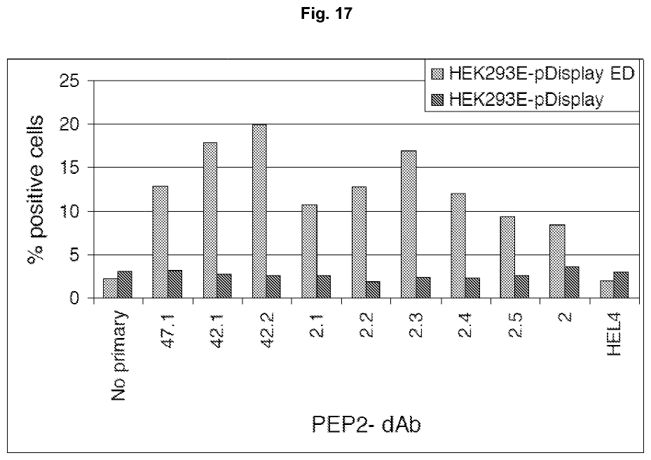

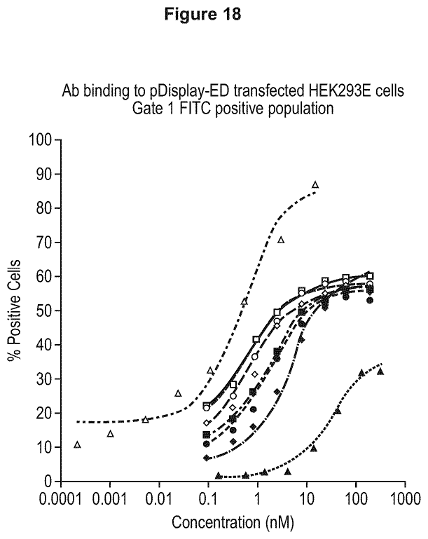

Binding by flow cytometry to live HEK293E cells expressing pDisplay-ECD2 was clearly improved (FIG. 17). Gating live cell binding with HEL4 as the control negative binder showed clear improvements with a higher percentage of positive cells detected with lead dAbs indicating the target epitope was less sterically hindered and available for binding (FIG. 18).

Conclusion: Antigen binding sites have been identified that bind the non-functional P2X₇ receptor on live cells and ECD2. The removal of residues 307-332, commencing an estimated 3 nm from the E200 epitope site, has improved binding with the removal of partial steric hindrance. No loss of E200 conformation occurs even though the segment 307-332 would be expected to stabilise the protein fold as it interacts closely with the N-terminal segment.

### Example 5

### Generating Various High Affinity Binders

Objective: To generate antigen binding sites with high affinity for the non-functional P2X₇ receptor.

Background: The antigen binding sites from Example 1 having the following sequences:

| | CDR1 CDR2 CDR3 |
|---|---|
| WT | SSYAMS---AISGSGGSTYYADSVKG---CAKSYGA--------FDY |
| PEP2-2 | RNHD.G---AISGSGGS.........---..EPKPMDTE-----..Y |
| PEP2-47 | PMKD.G---AISGSGGS.........---..EPSHFDRP-----..Y |
| PEP2-42 | DNVE.S---SIGSKGED.........---..QTVNVPEPA----.AY |
| PEP2-1 | DNEP.G---S.AD..NH.........---...QR.LNRYRAQ--..Y |
| PEP2-5 | PASN..---S.TA..YR.........---...QGQISNFPR---..Y |
| PEP2-4 | GM.N..---S.NAT..R.........---...FNRFSHRQYN--..Y |
| PEP2-34 | ......---T.TSD.LR.........---...VHTFANRSLN--..Y |
| PEP2-7 | GA.S..---T.N...LA.........---...CSSCTSLNAN--..Y |
| PEP2-11 | AR.P.A---S.D.G.LQ.........---...ASAPKYFR----..Y |
| PEP2-30 | AK.P.V---S.GPG.AR.........---...PWRVYSYDR---..Y |
| PEP2-13 | ...A.A---T.D.N.LI.........---...LQRYDRYTLN..Y |

were used as starting points for iterative rounds of randomization and screening subject to issues of binding in the Fc format, solubility and possession of a uniphasic dissociation trace on Biacore. PEP2-2 and PEP2-47 possessed the requisite characteristics and were selected for affinity maturation even though they surprisingly had lower single domain affinity for the ECD2 conformational and E200 peptide targets than other lead dabs such as PEP2-4 and PEP2-5

Materials and methods: The selected V_{H} domains including 2-2, 2-47 and daughters ere affinity matured through 6 rounds of sequence diversification that included all CDRs as well as all framework regions through NNS diversification that sampled all 20 amino acids at each position. The scaffold of the V_{H} library originated from the human V_{H} that gave rise to the HEL4 control non-binder and the diverse positive binders has the sequence:

VHD EVQLLEPGGGLVQPGGSLRLSCAASGVNVSHDSMTWVRQAPGKGLE
WVSAIRGPNGSTYYADSVKGRFTISRDNSKNTLYLQMNSLRAEDTAVYYC
ASGARHADTERPPSQQTMPFWGQGTLVTVSS

Error-prone libraries were generated with a 2.7 amino acid error rate. Pools of clones were screened against the E200 initially and then the ECD2 by phage ELISA for increased binding affinity. Eight error-prone libraries were subcloned into the soluble dAb expression vector pDOM38 without tag. Passive selection was carried out until Round 3. A total of 1000 clones were screened by Biacore from Round 5 libraries PEP2-42, PEP2-pooled and the Round 4 library PEP2-pooled. The pool of clones represents PEP2 clones 2-1, 2-2, 2-11, 2-13, 2-30, 2-34, 2-42 and 2-47. Improvement in off-rates by Biacore were observed. ELISA screening against 1 nM biotinylated E200 showed EC₅₀ improvement from the range 10⁷ to 10⁶ ug/mL in Round 3 to 10⁴ ug/mL in Round 5, well above control dAbs.

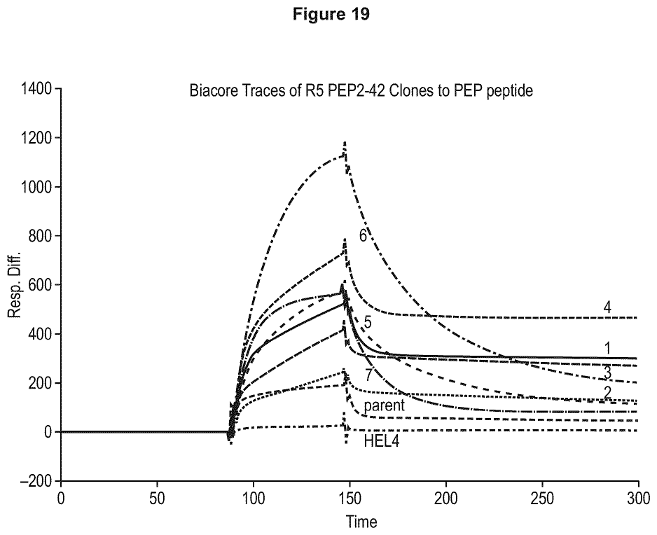

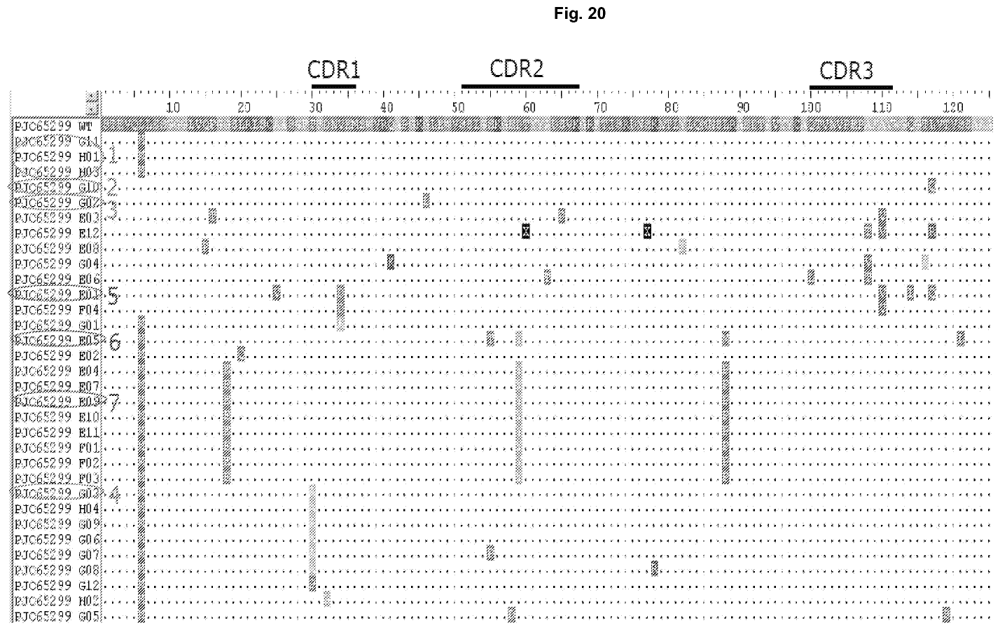

Biacore tracing of selected PEP2-42 clones to E200 peptide are shown in FIG. 19. The parent clone and HEL4 control dabs are at the bottom of the figure. Sequence variations of the selected clones are shown in the following figure. The 32 clones shown all have improved off-rates. Off-rate curves fell into two families and clones were chosen accordingly (FIG. 20) with E/F (blue at left) representing a classical off-rate curve and G/H (red at left) an irregular biphasic type. K_{D} values are 76 nM for clone 6 and 200 nM for clone 7.

Determination of biochemical and/or biophysical characteristics of the antigen binding sites were obtained by SEC-MALLS. Those with monomeric solution characteristics were selected over those with a propensity to aggregate. Clones were generally found with a solubility in PBS>10 mg/mL.

NNS screening, particularly of part of the variable CDR3 region, but extending to critical residues in F4 such as the residues 103-105 was used to refine antigen binding.

### Results

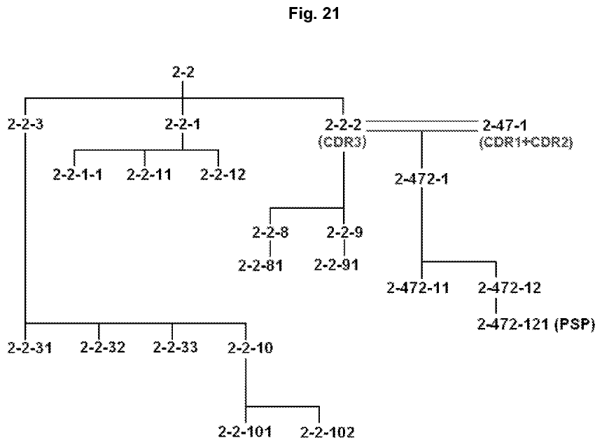

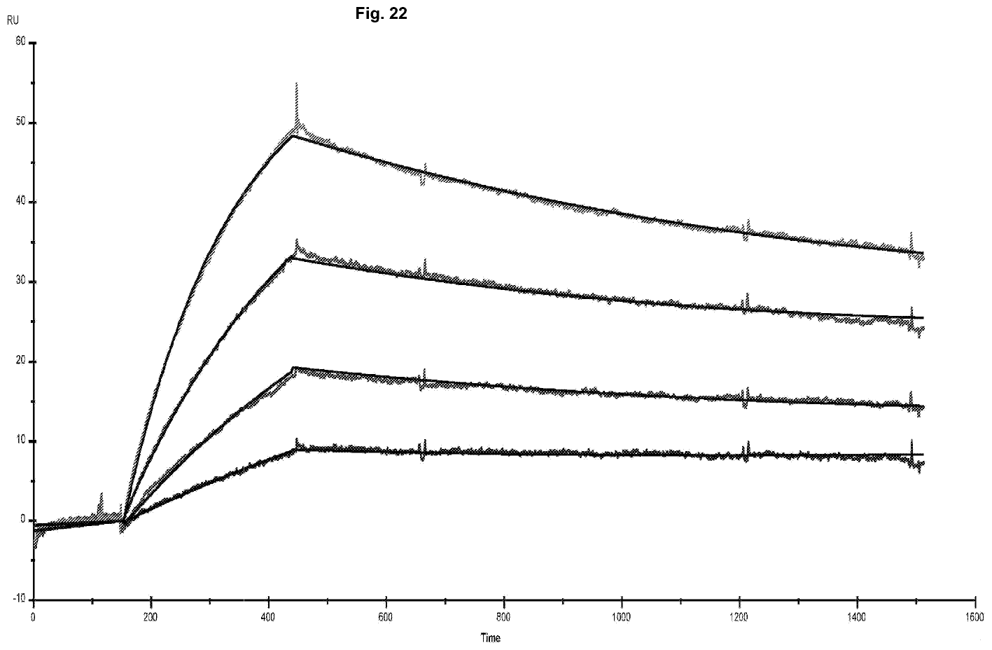

The affinity maturation family tree of antibodies is shown in the FIG. 21. An example of the improved binding by Biacore is shown in the form of the clone PEP2-2-3Fc in FIG. 22. The channel was coated with 10RU E200 peptide and then loaded with 100 pM, 250 pM, 500 pM and 1nM PEP2-2-3 in ascending order on the figure. Curve fitting reveals a K_{D} of 130 pM. The corresponding value for the unformatted dAb PEP2-2-3 against E200 is 7 nM, showing a more moderate increase in binding for the high affinity dabs when formatted as dAb-Fc compared with the increase from the parent dabs such as PEP2-2 that increased from 1 uM to 300 pM.

Corresponding values for the K_{D} when measured against ECD2 in either solution form or as a ECD-Fc construct showed significantly lower binding against the conformational epitope, with PEP2-2-3 Fc producing a value of 1.5 nM, PEP2-2-1 560 pM and PEP2-472-1 584 pM as examples.

Examples of PEP2-Fc KD derived from Biacore using E200 are shown in the following Table.

| PEP-Fc | K_{D} (pM) |
|---|---|
| 2-2 | 300 |
| 2-2-2 | 100 |
| 2-2-3 | 130 |
| 2-2-1-1 | 90 |
| 2-42 | 5,500 |
| 2-42-1 | 120 |
| 2-47 | 7500 |
| 2-47-1 | 110 |
| 2-247-1 | 190 |
| (2-2/2-47-1 CDR crossover) | |
| 2-247-2 | 450 |
| (2-2-1/2-47-1 CDR crossover) | |
| 2-472-1 | 90 |
| (2-47-1/2-2-2 CDR crossover) | |

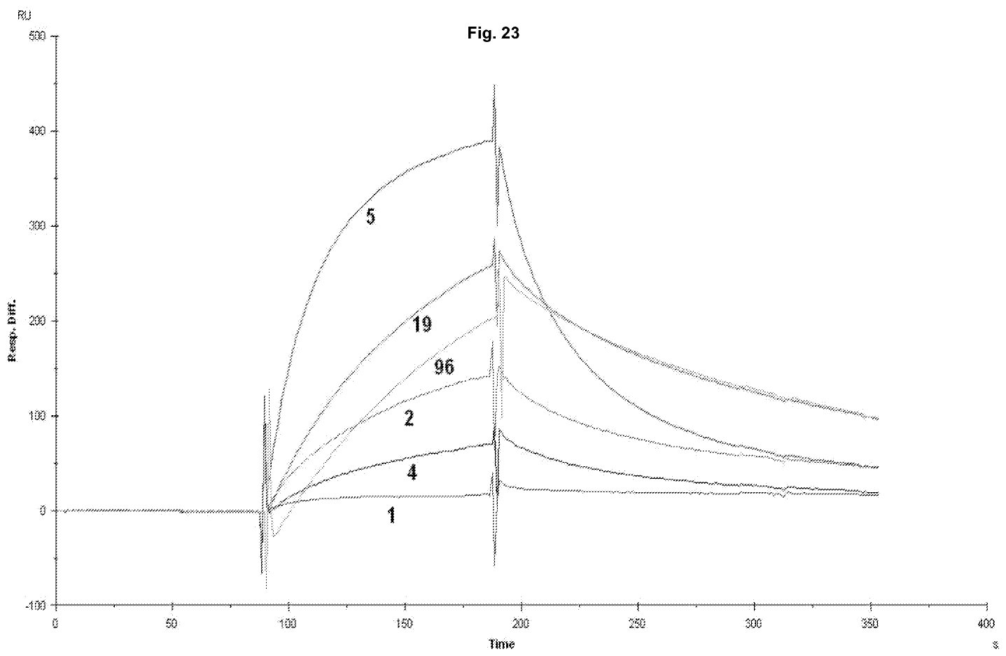

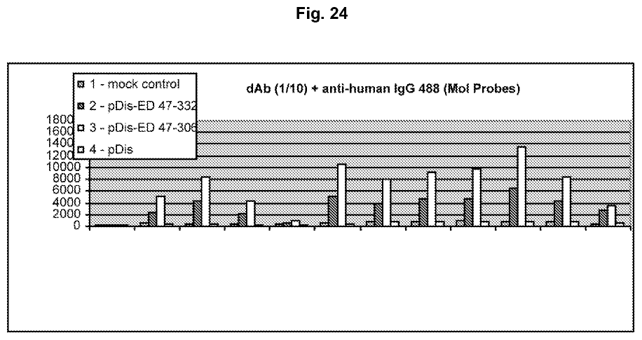

The effect of NNS screening on position 103 in PEP2-2-1 is shown in FIG. 23. Trace 1 is buffer only and Trace 5 is a typical example of improved binding obtained by exchanging the Trp for an Arg residue.

Binding of selected lead clones to HEK293 cells expressing mock control (no binding), pDisplay-ECD1 (moderate binding), pDisplay-ECD2 (higher binding) and pDisplay control (no binding) is seen in FIG. 24.

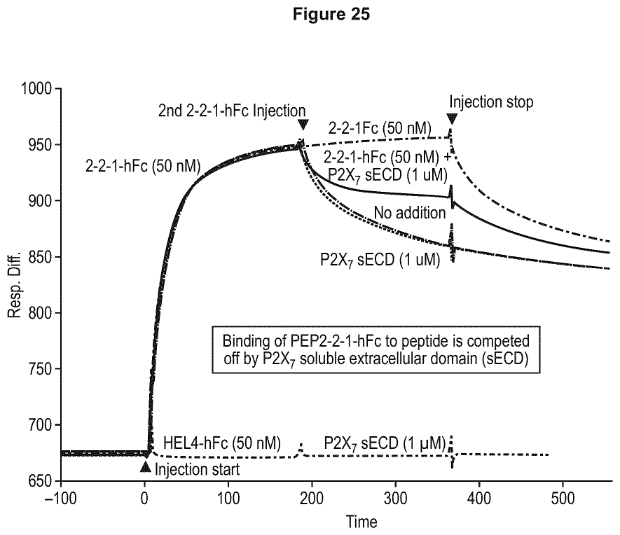

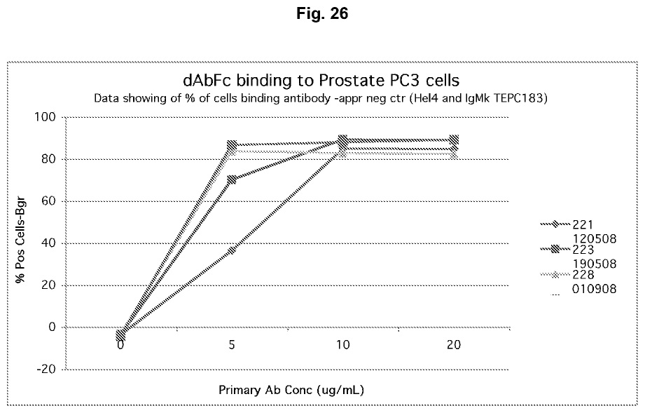

The lead clones bind specifically and competitively to the target antigen and can be competed off with the addition of the soluble ECD2. As an example FIG. 25 shows PEP2-2-1 Fc at 50 nM is competed off with 1 uM of soluble ECD2. An SA Biacore chip is coated with E200-biotin peptide. Data shown is from 20 RU coated channel with a flow rate of 20 uL/min in HBS-EP buffer. The HEL 4 Fc neither binds nor is affected by the addition of the ECD2. Similar results are achieved in competing off the PEP2-2-1 Fc with E200 at 5 uM or the ECD2 Fc construct at 1 uM.

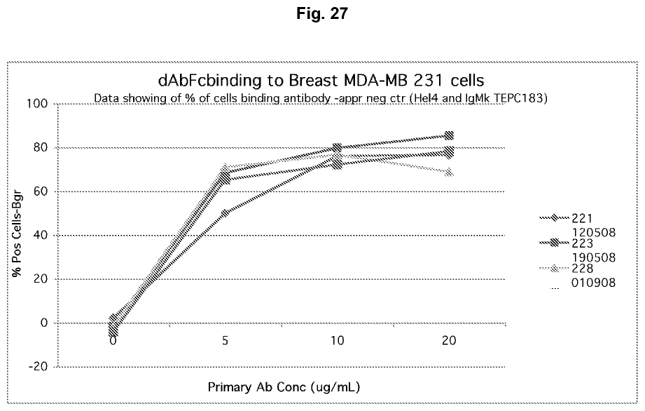

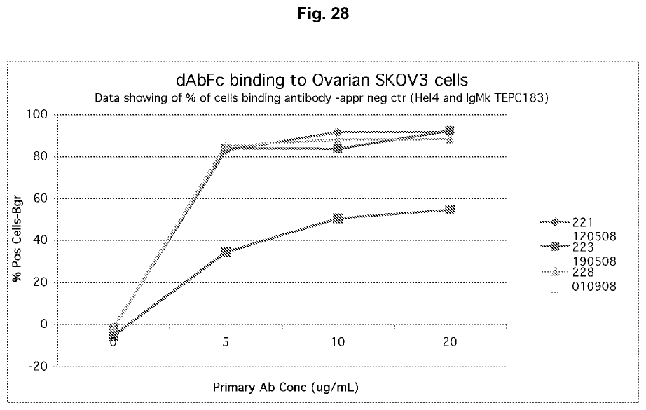

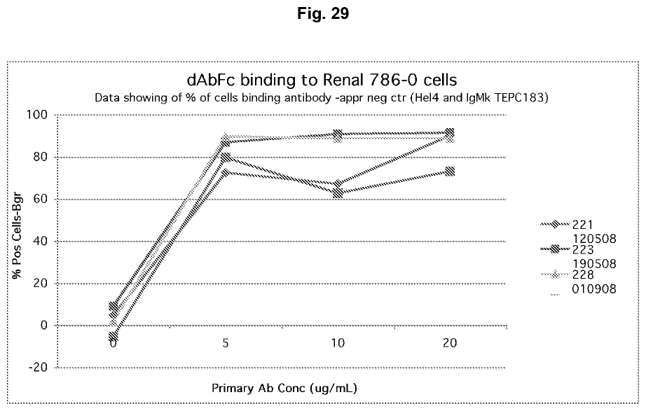

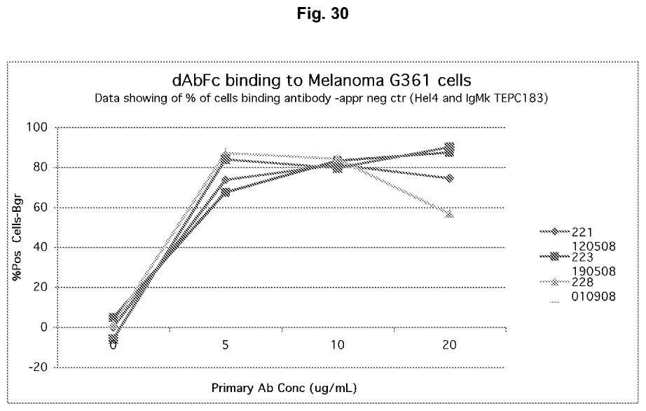

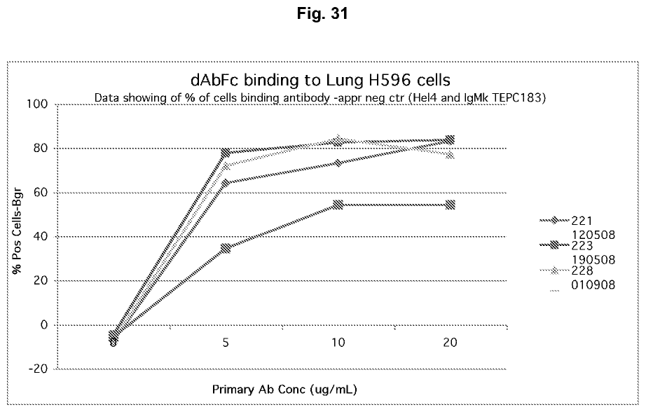

Flow cytometry of binding of several lead dAb-Fc antigen binders to live cancer cells is shown in the following examples. These include: prostate PC3 (FIG. 26), breast MDA-MB 231 (FIG. 27), ovarian SKOV-3 (FIG. 28), Renal 786-O (FIG. 29), Melanoma G361 (FIG. 30) and Lung NCI-H596 (FIG. 31) cell lines.

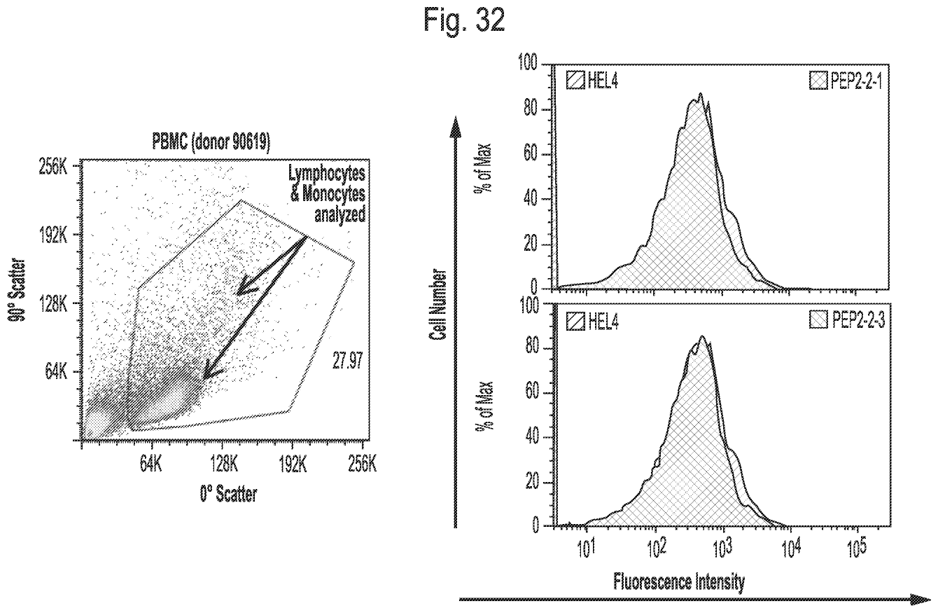

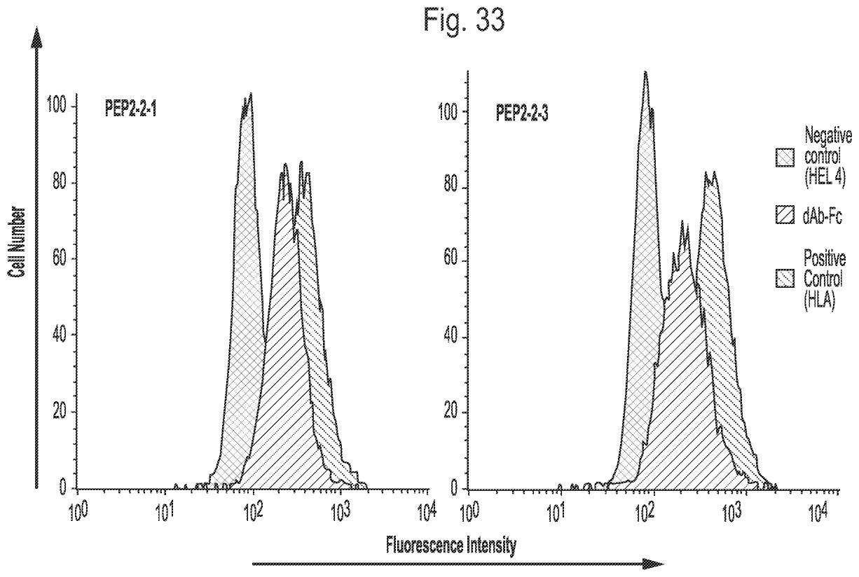

Non-crossreactivity with functional P2X₇ receptors on lymphocytes and monocytes was examined with flow cytometry. An example is shown in FIG. 32 in which the two dAb Fc clones PEP2-2-1 and PEP2-2-3 Fc showed no binding above the HEL4 Fc control background. In contrast, binding to live cancer cells such as prostate LNCap is clear (green in FIG. 33, with the HEL4 control in red showing no binding above the secondary and the HLA positive control shown in blue).

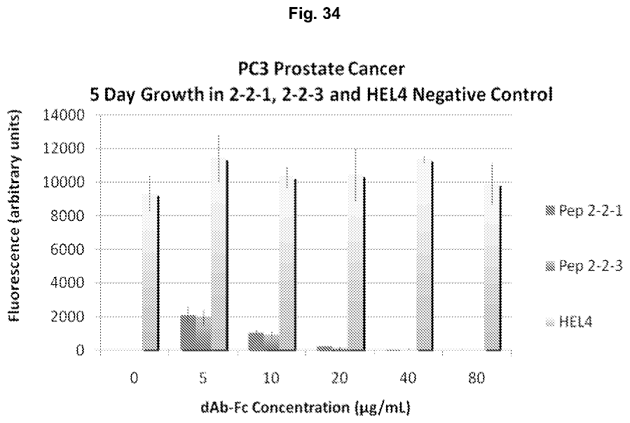

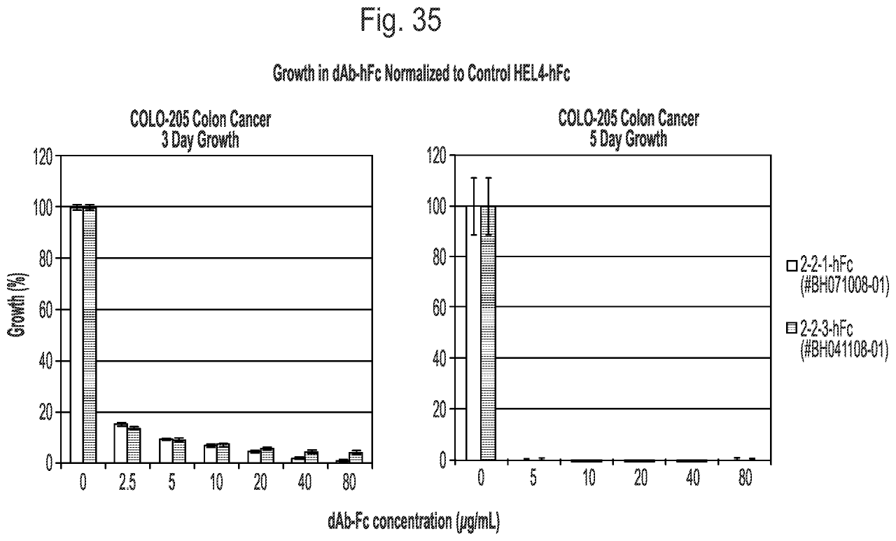

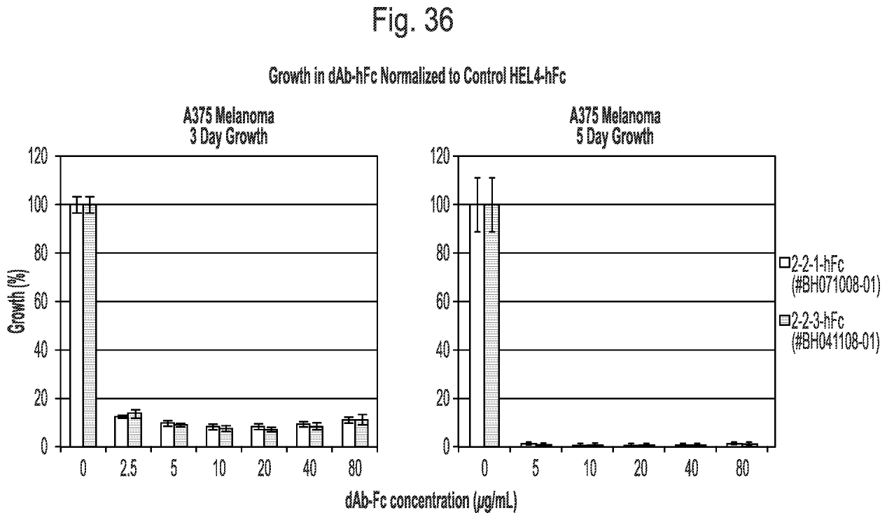

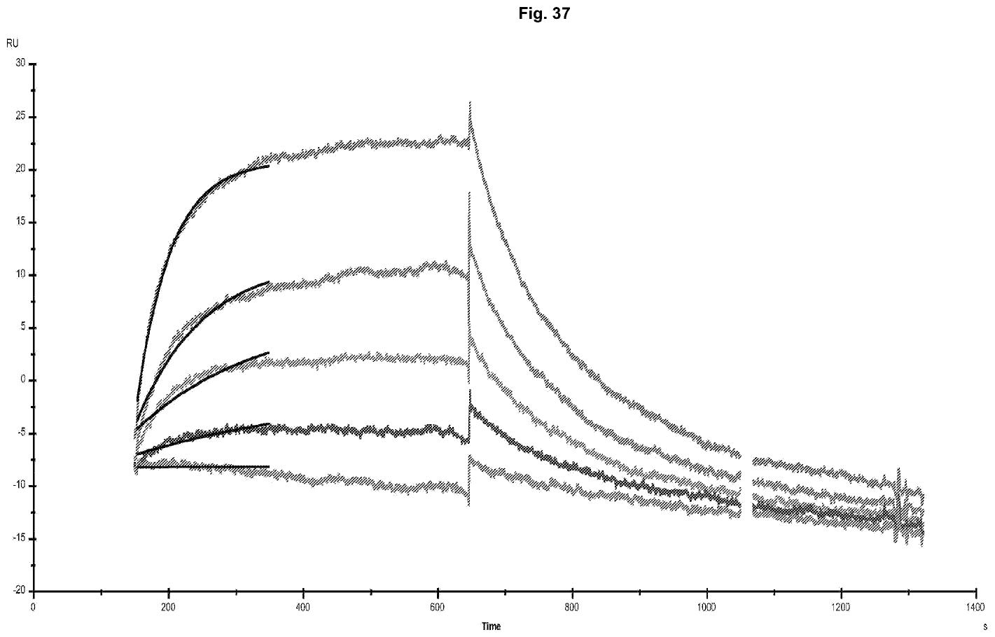

Direct cell killing or growth inhibition, as measured using the Cell Titer Blue Assay, was monitored with the lead clones PEP2-2-1 and PEP2-2-3 using a variety of cell lines. Over a 3 or 5 day growth cycle, the control cells grew while the net growth in the presence of the 2-2-1 Fc or 2-2-3 Fc was measured as a proportion of the growth in the presence of the HEL4 Fc control. FIG. 34 shows PC3 cell growth progressively inhibited as 2-2-1 or 2-2-3 are titrated up to 40 ug/mL over 5 days whereas the control cells are unaffected by HEL4 Fc. The colorectal cancer cell line COLO205 shows more sensitivity with both 2-2-1 and 2-2-3 Fc causing significant growth inhibition at 3 days while at 5 days, no cells remain even at 2.5 ug/mL (FIG. 35). Similarly the melanoma cell line A375 shows significant cell killing at 3 days while at 5 days no cells remain (FIG. 36).

Conclusion: Antigen binding sites that have high affinity for the non-functional P2X₇ receptor on live cells were identified, sequenced and biophysically characterised. Their effects on cell function were examined.

### Example 6

### Future Experiments

Objective: To further enhance affinity of the lead dabs through additional targeted NNS screening of residues involved in direct binding to the antigen and in residues enabling the CDRs to pack more efficiently. To improve stability and solubility of antigen binding sites by modifying the Fc. To improve the efficiency of cell killing.

Materials and methods: Standard techniques to enhance binding affinity such as additional rounds of NNS screening will be performed. The clones produced will be screened by Biacore to find those with improved off rates and phage ELISA against ECD2 (47-306). Additional screening using the CTB Assay will be performed to identify clones with the most efficient combination of binding affinity and killing capacity.

Expected results: Clones with at least one log lower binding constants are expected to be isolated that also kill cancer cells more efficiently than existing leads. As an example, new high affinity lead dAb domains (no Fc format) such as PEP2-2-12 in FIG. 36 show a KD against the ECD2 domain of 945 pM whereas the parent PEP2-2-1 exhibits a KD of 560 pM as an Fc construct with associated co-operative binding. The construction of leads with different Fc domains will enable the influence of the Fc on solubility properties and cell killing to be examined. Examples are the addition of mouse type IgG2a Fc in place of human IgG type 1 Fc.

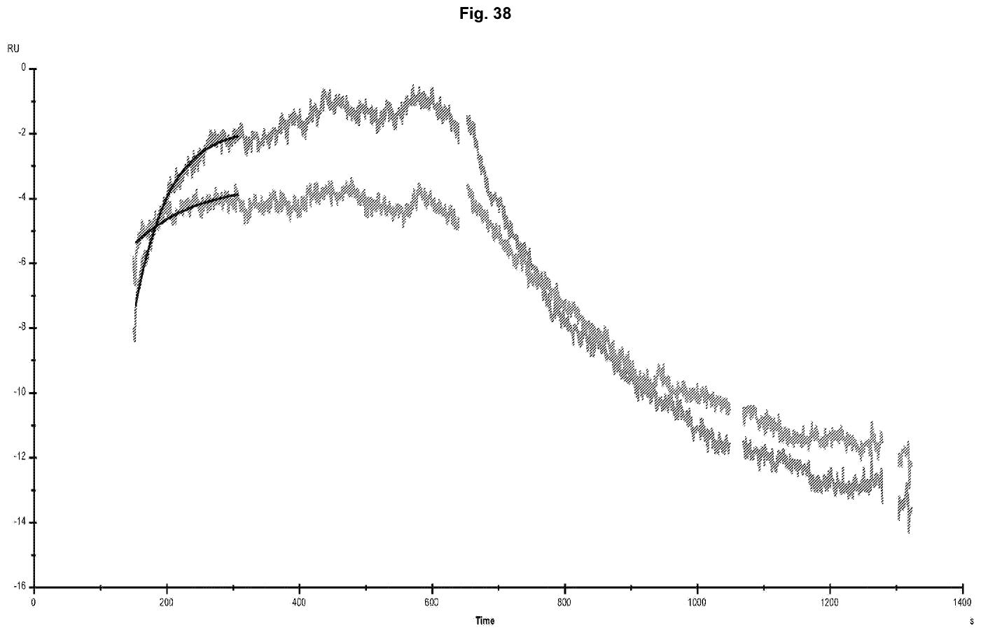

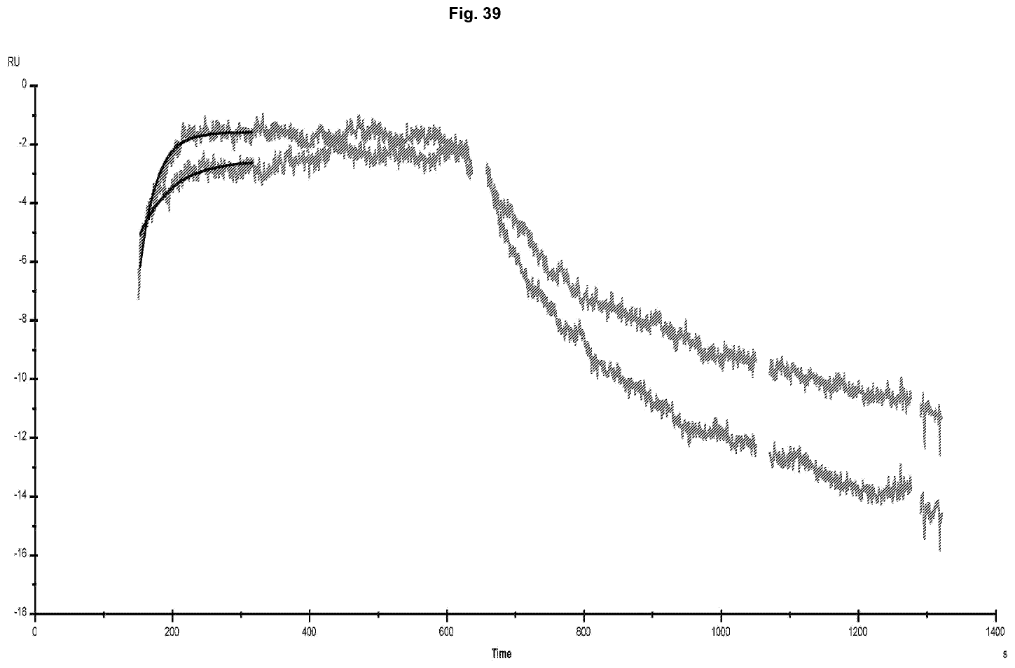

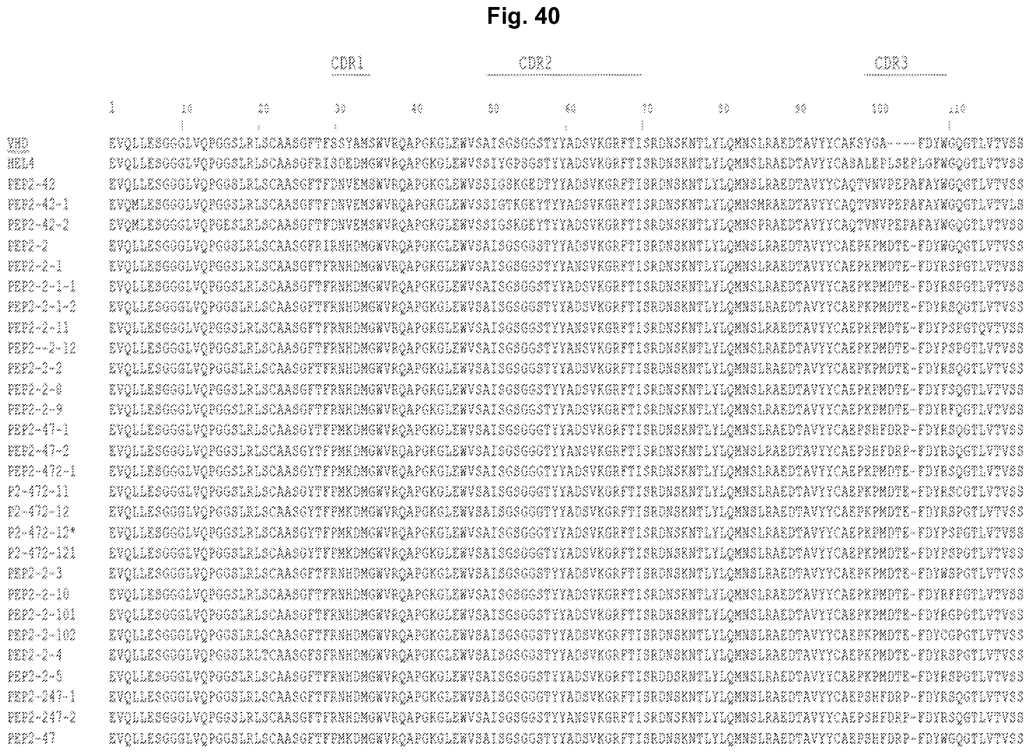

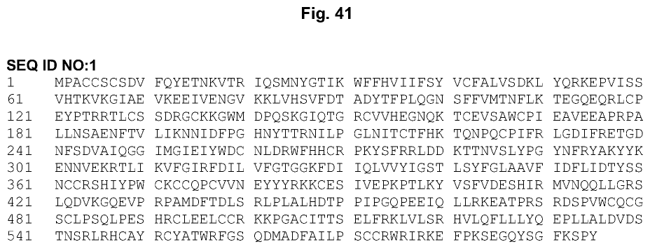

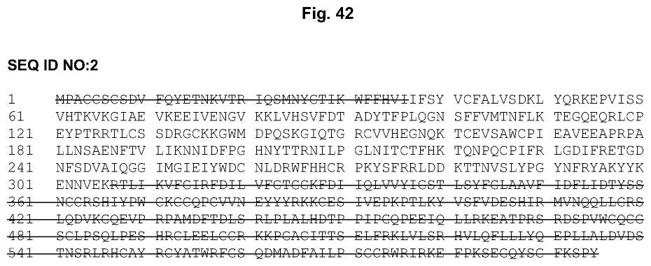

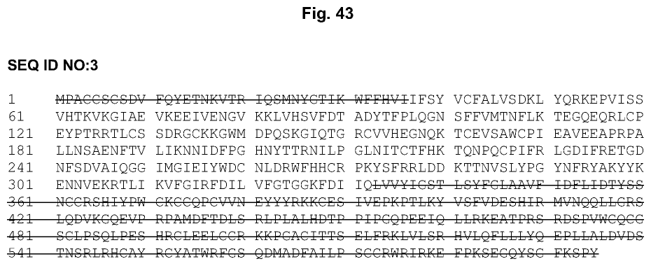

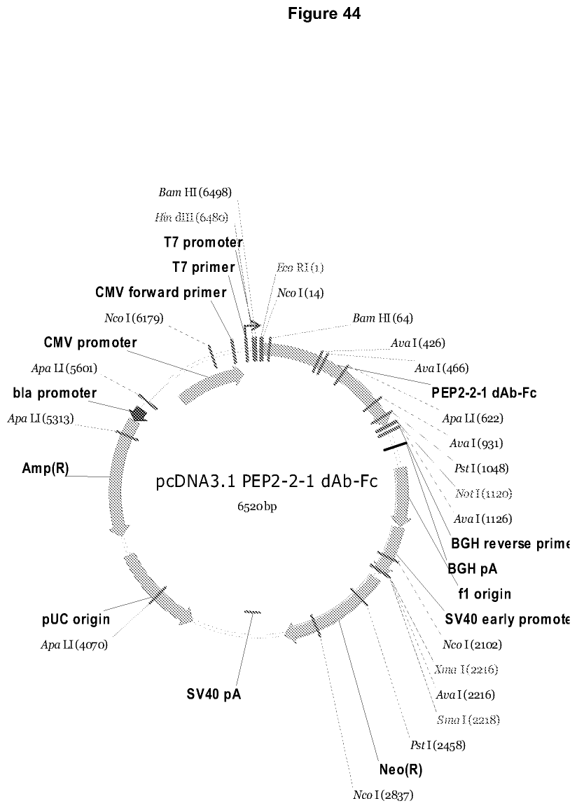

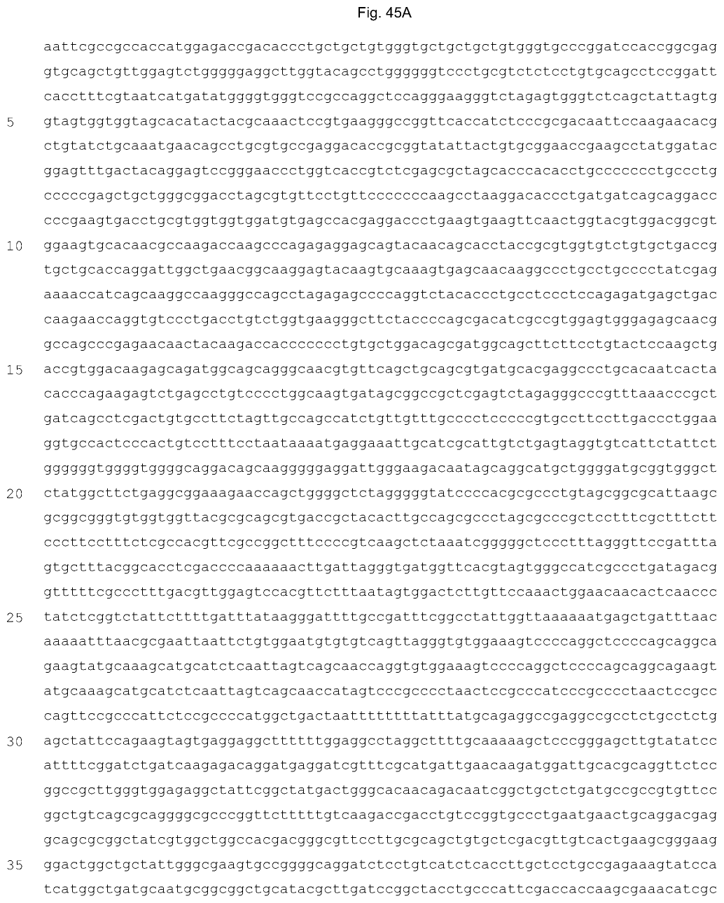

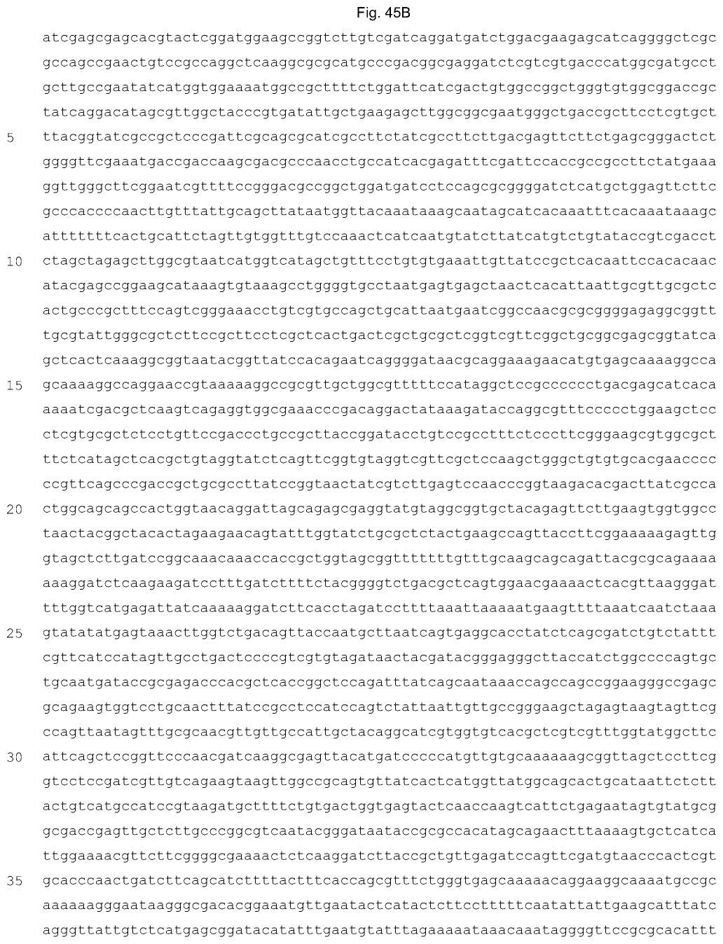

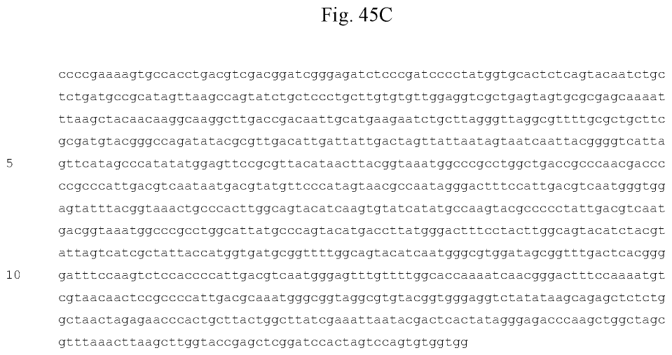

The labelling of high affinity single domain species would enable them to be used for systemic screening purposes. An example is shown in FIG. 38 in which an Alexa488 label has been attached to the dAb domain PEP2-2-12 and similar Biacore affinity determination suggests a K_{D} of 174 pM. A high affinity lead with different parent is shown in FIG. 39 where PEP2-472-12Alexa488 domain is measured with a KD of 156 pM.

1. An antigen binding site for binding to a P2X₇ receptor, the antigen binding site being defined by general formula 1:
FR1-CDR1-FR2-CDR2-FR3-CDR3-FR4
wherein:
FR1, FR2, FR3 and FR4 are each framework regions;
CDR1, CDR2 and CDR3 are each complementarity determining regions;
wherein:
CDR1 has a sequence selected from the group
consisting of: DNEPMG, RNHDMG, SGYAMA, GMYNMS,
PASNMS, GSYAMA, GAYAMS, DGYNMS, TYDMAW,
QEYGMG, ARYPMA, SSYAMA, AKYPMV, SSYAMS,
DNVEMS and PMKDMG.

2. An antigen binding site for binding to a P2X₇ receptor, the antigen binding site being defined by general formula 2:
FR1-CDR1-FR2-CDR2-FR3-CDR3-FR4
wherein:
FR1, FR2, FR3 and FR4 are each framework regions;
CDR1, CDR2 and CDR3 are each complementarity determining regions;
wherein:
CDR2 has a sequence selected from the
group consisting of: SIADSGNHTYYADSVKG,
AISGSGGSTYYADSVKG, TILSDGSRTYYADSVKG,
SINATGGRTYYADSVKG, SITASGYRTYYADSVKG,
TISTSGSSTYYADSVKG, TINGSGLATYYADSVKG,
SITANGNSTYYADSVKG, SIAAAGSRTYYADSVKG,
SITPSGDKTYYADSVKG, SIDGGGLQTYYADSVKG,
TIDGNGLITYYADSVKG, SIGPGGARTYYADSVKG,
TITSDGLRTYYADSVKG, SIGSKGEDTYYADSVKG,
AISGSGGSTYYANSVKG, AISGSGGGTYYADSVKG,
SIGTKGEYTYYADSVKG, SIGSKGEYTYYADSVKG and
AISGSGGGTYYANSVKG.

3. An antigen binding site for binding to a P2X₇ receptor, the antigen binding site being defined by general formula 3:
FR1-CDR1-FR2-CDR2-FR3-CDR3-FR4
wherein:
FR1, FR2, FR3 and FR4 are each framework regions;
CDR1, CDR2 and CDR3 are each complementarity determining regions;
wherein:
CDR3 has a sequence selected from the
group consisting of: KQRGLNRYRAQFDY, EPKPMDTEFDY,
KIKTFRNHSVQFDY, KFNGFSHRQYNFDY, KQGQISNFPRFDY,
KVRFATSKSINFDY, KCSSCTSLNANFDY, KASYSRPYNFQFDY,
KQRSISIRPMFDY, KVRSMSYAHFDFDY, KASAPKYFRFDY,
KLQRYDRYTLNFDY, KPWRVYSYDRFDY, KVHTFANRSLNFDY,
QTVNVPEPAFAY and EPSHFDRPFDY.

4-11. (canceled)

12. An antigen binding site or a CDR and/or FR sequence according claim 1 including one or more mutations for increasing the affinity of said site for binding to a P2X₇ receptor.

13. An antigen binding site or a CDR and/or FR sequence according to claim 1, wherein an amino acid sequence forming one or more of FR1, CDR1, FR2, CDR2, FR3, CDR3 and FR4 is a human sequence.

14. An anti P2X₇ receptor immunoglobulin variable domain, antibody, Fab, dab, scFv including an antigen binding site or including a CDR and/or FR sequence according to claim 1.

15. A diabody or triabody including an antigen binding site or a CDR and/or FR sequence according to claim 1.

16. A fusion protein including an antigen binding site, immunoglobulin variable domain, antibody, Fab, dab, scFv, diabody or triabody according to claim 1.

17. A conjugate in the form of an antigen binding site, immunoglobulin variable domain, antibody, Fab, dab, scFv, diabody, triabody or fusion protein according to claim 1 conjugated to a label or a cytotoxic agent.

18. An antibody for binding to an antigen binding site of an immunoglobulin variable domain, antibody, Fab, dab, scFv, diabody, triabody, fusion protein, or conjugate according to claim 1.

19. A nucleic acid encoding an antigen binding site, or a CDR and/or FR sequence, or an immunoglobulin variable domain, antibody, Fab, dab, scFv, diabody, triabody, fusion protein or conjugate according to claim 1.

20. A vector including a nucleic acid according to claim 19.

21. A cell including a vector or nucleic acid according to claim 1.

22. An animal or tissue derived therefrom including a cell according to claim 20.

23. A pharmaceutical composition including an antigen binding site, or including a CDR and/or FR sequence, or an immunoglobulin variable domain, antibody, Fab, dab, scFv, diabody, triabody, fusion protein, or conjugate according to claim 1 and a pharmaceutically acceptable carrier, diluent or excipient.

24. A diagnostic composition including an antigen binding site, or including a CDR and/or FR sequence or an immunoglobulin variable domain, antibody, Fab, dab, scFv, diabody, triabody, fusion protein or conjugate according to claim 1, a diluent and optionally a label.

25. A kit or article of manufacture including an antigen binding site, or including a CDR and/or FR sequence or an immunoglobulin variable domain, antibody, Fab, dab, scFv, diabody, triabody, fusion protein or conjugate according to claim 1.

26-27. (canceled)

28. A library of nucleic acid molecules produced from the mutation of an antigen binding site or a CDR and/or FR sequence according to claim 1, wherein at least one nucleic acid molecule in said library encodes an antigen binding site for binding to an a P2X₇ receptor.

29. A method for producing an anti P2X₇ antigen binding site according to claim 1 including expressing a nucleic acid in a cell or animal.

30. A method for the treatment of cancer or a condition or disease associated with expression of non functional P2X₇ receptor in an individual including the step of providing an antigen binding site, immunoglobulin variable domain, antibody, Fab, dab, scFv, diabody, triabody, fusion protein, conjugate or pharmaceutical composition according to claim 1 to an individual requiring treatment for cancer or said condition or disease.

31-32. (canceled)


SEQUENCE LISTING (224 sequences)

SEQ ID NO: 1 - protein, 595 aa, Homo sapiens [sequence omitted]

SEQ ID NO: 2 - protein, 270 aa, Homo sapiens
IFSYVCFALVSDKLYQRKEPVISSVHTKVKGIAEVKEEIVENGVKKLVHSVFDTADYTFPLQGNSFFVMTNFLKTEGQEQRLCPEYPTRRTLCSSDRGCKKGWMDPQSKGIQTGRCVVHEGNQKTCEVSAWCPIEAVEEAPRPALLNSAENFTVLIKNNIDFPGHNYTTRNILPGLNITCTFHKTQNPQCPIFRLGDIFRETGDNFSDVAIQGGIMGIEIYWDCNLDRWFHHCRPKYSFRRLDDKTTNVSLYPGYNFRYAKYYKENNVEK

SEQ ID NO: 3 - protein, 296 aa, Homo sapiens
IFSYVCFALVSDKLYQRKEPVISSVHTKVKGIAEVKEEIVENGVKKLVHSVFDTADYTFPLQGNSFFVMTNFLKTEGQEQRLCPEYPTRRTLCSSDRGCKKGWMDPQSKGIQTGRCVVHEGNQKTCEVSAWCPIEAVEEAPRPALLNSAENFTVLIKNNIDFPGHNYTTRNILPGLNITCTFHKTQNPQCPIFRLGDIFRETGDNFSDVAIQGGIMGIEIYWDCNLDRWFHHCRPKYSFRRLDDKTTNVSLYPGYNFRYAKYYKENNVEKRTLIKVFGIRFDILVFGTGGKFDIIQ

SEQ ID NO: 4 - protein, 6 aa, synthetic construct
  REGION 1..6: note = Description of Artificial Sequence: Synthetic peptide
DNEPMG

SEQ ID NO: 5 - protein, 17 aa, synthetic construct
  REGION 1..17: note = Description of Artificial Sequence: Synthetic peptide
SIADSGNHTYYADSVKG

SEQ ID NO: 6 - protein, 14 aa, synthetic construct
  REGION 1..14: note = Description of Artificial Sequence: Synthetic peptide
KQRGLNRYRAQFDY

SEQ ID NO: 7 - protein, 6 aa, synthetic construct
  REGION 1..6: note = Description of Artificial Sequence: Synthetic peptide
RNHDMG

SEQ ID NO: 8 - protein, 17 aa, synthetic construct
  REGION 1..17: note = Description of Artificial Sequence: Synthetic peptide
AISGSGGSTYYADSVKG

SEQ ID NO: 9 - protein, 11 aa, synthetic construct
  REGION 1..11: note = Description of Artificial Sequence: Synthetic peptide
EPKPMDTEFDY

SEQ ID NO: 10 - protein, 6 aa, synthetic construct
  REGION 1..6: note = Description of Artificial Sequence: Synthetic peptide
SGYAMA

SEQ ID NO: 11 - protein, 17 aa, synthetic construct
  REGION 1..17: note = Description of Artificial Sequence: Synthetic peptide
TILSDGSRTYYADSVKG

SEQ ID NO: 12 - protein, 14 aa, synthetic construct
  REGION 1..14: note = Description of Artificial Sequence: Synthetic peptide
KIKTFRNHSVQFDY

SEQ ID NO: 13 - protein, 6 aa, synthetic construct
  REGION 1..6: note = Description of Artificial Sequence: Synthetic peptide
GMYNMS

SEQ ID NO: 14 - protein, 17 aa, synthetic construct
  REGION 1..17: note = Description of Artificial Sequence: Synthetic peptide
SINATGGRTYYADSVKG

SEQ ID NO: 15 - protein, 14 aa, synthetic construct
  REGION 1..14: note = Description of Artificial Sequence: Synthetic peptide
KFNGFSHRQYNFDY

SEQ ID NO: 16 - protein, 6 aa, synthetic construct
  REGION 1..6: note = Description of Artificial Sequence: Synthetic peptide
PASNMS

SEQ ID NO: 17 - protein, 17 aa, synthetic construct
  REGION 1..17: note = Description of Artificial Sequence: Synthetic peptide
SITASGYRTYYADSVKG

SEQ ID NO: 18 - protein, 13 aa, synthetic construct
  REGION 1..13: note = Description of Artificial Sequence: Synthetic peptide
KQGQISNFPRFDY

SEQ ID NO: 19 - protein, 6 aa, synthetic construct
  REGION 1..6: note = Description of Artificial Sequence: Synthetic peptide
GSYAMA

SEQ ID NO: 20 - protein, 17 aa, synthetic construct
  REGION 1..17: note = Description of Artificial Sequence: Synthetic peptide
TISTSGSSTYYADSVKG

SEQ ID NO: 21 - protein, 14 aa, synthetic construct
  REGION 1..14: note = Description of Artificial Sequence: Synthetic peptide
KVRFATSKSINFDY

SEQ ID NO: 22 - protein, 6 aa, synthetic construct
  REGION 1..6: note = Description of Artificial Sequence: Synthetic peptide
GAYAMS

SEQ ID NO: 23 - protein, 17 aa, synthetic construct
  REGION 1..17: note = Description of Artificial Sequence: Synthetic peptide
TINGSGLATYYADSVKG

SEQ ID NO: 24 - protein, 14 aa, synthetic construct
  REGION 1..14: note = Description of Artificial Sequence: Synthetic peptide
KCSSCTSLNANFDY

SEQ ID NO: 25 - protein, 6 aa, synthetic construct
  REGION 1..6: note = Description of Artificial Sequence: Synthetic peptide
DGYNMS

SEQ ID NO: 26 - protein, 17 aa, synthetic construct
  REGION 1..17: note = Description of Artificial Sequence: Synthetic peptide
SITANGNSTYYADSVKG

SEQ ID NO: 27 - protein, 14 aa, synthetic construct
  REGION 1..14: note = Description of Artificial Sequence: Synthetic peptide
KASYSRPYNFQFDY

SEQ ID NO: 28 - protein, 6 aa, synthetic construct
  REGION 1..6: note = Description of Artificial Sequence: Synthetic peptide
TYDMAW

SEQ ID NO: 29 - protein, 17 aa, synthetic construct
  REGION 1..17: note = Description of Artificial Sequence: Synthetic peptide
SIAAAGSRTYYADSVKG

SEQ ID NO: 30 - protein, 13 aa, synthetic construct
  REGION 1..13: note = Description of Artificial Sequence: Synthetic peptide
KQRSISIRPMFDY

SEQ ID NO: 31 - protein, 6 aa, synthetic construct
  REGION 1..6: note = Description of Artificial Sequence: Synthetic peptide
QEYGMG

SEQ ID NO: 32 - protein, 17 aa, synthetic construct
  REGION 1..17: note = Description of Artificial Sequence: Synthetic peptide
SITPSGDKTYYADSVKG

SEQ ID NO: 33 - protein, 14 aa, synthetic construct
  REGION 1..14: note = Description of Artificial Sequence: Synthetic peptide
KVRSMSYAHFDFDY

SEQ ID NO: 34 - protein, 6 aa, synthetic construct
  REGION 1..6: note = Description of Artificial Sequence: Synthetic peptide
ARYPMA

SEQ ID NO: 35 - protein, 17 aa, synthetic construct
  REGION 1..17: note = Description of Artificial Sequence: Synthetic peptide
SIDGGGLQTYYADSVKG

SEQ ID NO: 36 - protein, 12 aa, synthetic construct
  REGION 1..12: note = Description of Artificial Sequence: Synthetic peptide
KASAPKYFRFDY

SEQ ID NO: 37 - protein, 6 aa, synthetic construct
  REGION 1..6: note = Description of Artificial Sequence: Synthetic peptide
SSYAMA

SEQ ID NO: 38 - protein, 17 aa, synthetic construct
  REGION 1..17: note = Description of Artificial Sequence: Synthetic peptide
TIDGNGLITYYADSVKG

SEQ ID NO: 39 - protein, 14 aa, synthetic construct
  REGION 1..14: note = Description of Artificial Sequence: Synthetic peptide
KLQRYDRYTLNFDY

SEQ ID NO: 40 - protein, 6 aa, synthetic construct
  REGION 1..6: note = Description of Artificial Sequence: Synthetic peptide
AKYPMV

SEQ ID NO: 41 - protein, 17 aa, synthetic construct
  REGION 1..17: note = Description of Artificial Sequence: Synthetic peptide
SIGPGGARTYYADSVKG

SEQ ID NO: 42 - protein, 13 aa, synthetic construct
  REGION 1..13: note = Description of Artificial Sequence: Synthetic peptide
KPWRVYSYDRFDY

SEQ ID NO: 43 - protein, 6 aa, synthetic construct
  REGION 1..6: note = Description of Artificial Sequence: Synthetic peptide
SSYAMS

SEQ ID NO: 44 - protein, 17 aa, synthetic construct
  REGION 1..17: note = Description of Artificial Sequence: Synthetic peptide
TITSDGLRTYYADSVKG

SEQ ID NO: 45 - protein, 14 aa, synthetic construct
  REGION 1..14: note = Description of Artificial Sequence: Synthetic peptide
KVHTFANRSLNFDY

SEQ ID NO: 46 - protein, 6 aa, synthetic construct
  REGION 1..6: note = Description of Artificial Sequence: Synthetic peptide
DNVEMS

SEQ ID NO: 47 - protein, 17 aa, synthetic construct
  REGION 1..17: note = Description of Artificial Sequence: Synthetic peptide
SIGSKGEDTYYADSVKG

SEQ ID NO: 48 - protein, 12 aa, synthetic construct
  REGION 1..12: note = Description of Artificial Sequence: Synthetic peptide
QTVNVPEPAFAY

SEQ ID NO: 49 - protein, 6 aa, synthetic construct
  REGION 1..6: note = Description of Artificial Sequence: Synthetic peptide
PMKDMG

SEQ ID NO: 50 - protein, 17 aa, synthetic construct
  REGION 1..17: note = Description of Artificial Sequence: Synthetic peptide
AISGSGGSTYYADSVKG

SEQ ID NO: 51 - protein, 11 aa, synthetic construct
  REGION 1..11: note = Description of Artificial Sequence: Synthetic peptide
EPSHFDRPFDY

SEQ ID NO: 52 - protein, 6 aa, synthetic construct
  REGION 1..6: note = Description of Artificial Sequence: Synthetic peptide
RNHDMG

SEQ ID NO: 53 - protein, 17 aa, synthetic construct
  REGION 1..17: note = Description of Artificial Sequence: Synthetic peptide
AISGSGGSTYYANSVKG

SEQ ID NO: 54 - protein, 11 aa, synthetic construct
  REGION 1..11: note = Description of Artificial Sequence: Synthetic peptide
EPKPMDTEFDY

SEQ ID NO: 55 - protein, 6 aa, synthetic construct
  REGION 1..6: note = Description of Artificial Sequence: Synthetic peptide
RNHDMG

SEQ ID NO: 56 - protein, 17 aa, synthetic construct
  REGION 1..17: note = Description of Artificial Sequence: Synthetic peptide
AISGSGGSTYYADSVKG

SEQ ID NO: 57 - protein, 11 aa, synthetic construct
  REGION 1..11: note = Description of Artificial Sequence: Synthetic peptide
EPKPMDTEFDY

SEQ ID NO: 58 - protein, 6 aa, synthetic construct
  REGION 1..6: note = Description of Artificial Sequence: Synthetic peptide
RNHDMG

SEQ ID NO: 59 - protein, 17 aa, synthetic construct
  REGION 1..17: note = Description of Artificial Sequence: Synthetic peptide
AISGSGGSTYYADSVKG

SEQ ID NO: 60 - protein, 11 aa, synthetic construct
  REGION 1..11: note = Description of Artificial Sequence: Synthetic peptide
EPKPMDTEFDY

SEQ ID NO: 61 - protein, 6 aa, synthetic construct
  REGION 1..6: note = Description of Artificial Sequence: Synthetic peptide
RNHDMG

SEQ ID NO: 62 - protein, 17 aa, synthetic construct
  REGION 1..17: note = Description of Artificial Sequence: Synthetic peptide
AISGSGGSTYYANSVKG

SEQ ID NO: 63 - protein, 11 aa, synthetic construct
  REGION 1..11: note = Description of Artificial Sequence: Synthetic peptide
EPKPMDTEFDY

SEQ ID NO: 64 - protein, 6 aa, synthetic construct
  REGION 1..6: note = Description of Artificial Sequence: Synthetic peptide
RNHDMG

SEQ ID NO: 65 - protein, 17 aa, synthetic construct
  REGION 1..17: note = Description of Artificial Sequence: Synthetic peptide
AISGSGGSTYYANSVKG

SEQ ID NO: 66 - protein, 11 aa, synthetic construct
  REGION 1..11: note = Description of Artificial Sequence: Synthetic peptide
EPKPMDTEFDY

SEQ ID NO: 67 - protein, 6 aa, synthetic construct
  REGION 1..6: note = Description of Artificial Sequence: Synthetic peptide
RNHDMG

SEQ ID NO: 68 - protein, 17 aa, synthetic construct
  REGION 1..17: note = Description of Artificial Sequence: Synthetic peptide
AISGSGGSTYYADSVKG

SEQ ID NO: 69 - protein, 11 aa, synthetic construct
  REGION 1..11: note = Description of Artificial Sequence: Synthetic peptide
EPKPMDTEFDY

SEQ ID NO: 70 - protein, 6 aa, synthetic construct
  REGION 1..6: note = Description of Artificial Sequence: Synthetic peptide
RNHDMG

SEQ ID NO: 71 - protein, 17 aa, synthetic construct
  REGION 1..17: note = Description of Artificial Sequence: Synthetic peptide
AISGSGGSTYYADSVKG

SEQ ID NO: 72 - protein, 11 aa, synthetic construct
  REGION 1..11: note = Description of Artificial Sequence: Synthetic peptide
EPKPMDTEFDY

SEQ ID NO: 73 - protein, 6 aa, synthetic construct
  REGION 1..6: note = Description of Artificial Sequence: Synthetic peptide
RNHDMG

SEQ ID NO: 74 - protein, 17 aa, synthetic construct
  REGION 1..17: note = Description of Artificial Sequence: Synthetic peptide
AISGSGGSTYYADSVKG

SEQ ID NO: 75 - protein, 11 aa, synthetic construct
  REGION 1..11: note = Description of Artificial Sequence: Synthetic peptide
EPKPMDTEFDY

SEQ ID NO: 76 - protein, 6 aa, synthetic construct
  REGION 1..6: note = Description of Artificial Sequence: Synthetic peptide
RNHDMG

SEQ ID NO: 77 - protein, 17 aa, synthetic construct
  REGION 1..17: note = Description of Artificial Sequence: Synthetic peptide
AISGSGGSTYYADSVKG

SEQ ID NO: 78 - protein, 11 aa, synthetic construct
  REGION 1..11: note = Description of Artificial Sequence: Synthetic peptide
EPKPMDTEFDY

SEQ ID NO: 79 - protein, 6 aa, synthetic construct
  REGION 1..6: note = Description of Artificial Sequence: Synthetic peptide
RNHDMG

SEQ ID NO: 80 - protein, 17 aa, synthetic construct
  REGION 1..17: note = Description of Artificial Sequence: Synthetic peptide
AISGSGGSTYYADSVKG

SEQ ID NO: 81 - protein, 11 aa, synthetic construct
  REGION 1..11: note = Description of Artificial Sequence: Synthetic peptide
EPKPMDTEFDY

SEQ ID NO: 82 - protein, 6 aa, synthetic construct
  REGION 1..6: note = Description of Artificial Sequence: Synthetic peptide
RNHDMG

SEQ ID NO: 83 - protein, 17 aa, synthetic construct
  REGION 1..17: note = Description of Artificial Sequence: Synthetic peptide
AISGSGGSTYYADSVKG

SEQ ID NO: 84 - protein, 11 aa, synthetic construct
  REGION 1..11: note = Description of Artificial Sequence: Synthetic peptide
EPKPMDTEFDY

SEQ ID NO: 85 - protein, 6 aa, synthetic construct
  REGION 1..6: note = Description of Artificial Sequence: Synthetic peptide
RNHDMG

SEQ ID NO: 86 - protein, 17 aa, synthetic construct
  REGION 1..17: note = Description of Artificial Sequence: Synthetic peptide
AISGSGGSTYYADSVKG

SEQ ID NO: 87 - protein, 11 aa, synthetic construct
  REGION 1..11: note = Description of Artificial Sequence: Synthetic peptide
EPKPMDTEFDY

SEQ ID NO: 88 - protein, 6 aa, synthetic construct
  REGION 1..6: note = Description of Artificial Sequence: Synthetic peptide
RNHDMG

SEQ ID NO: 89 - protein, 17 aa, synthetic construct
  REGION 1..17: note = Description of Artificial Sequence: Synthetic peptide
AISGSGGSTYYADSVKG

SEQ ID NO: 90 - protein, 11 aa, synthetic construct
  REGION 1..11: note = Description of Artificial Sequence: Synthetic peptide
EPKPMDTEFDY

SEQ ID NO: 91 - protein, 6 aa, synthetic construct
  REGION 1..6: note = Description of Artificial Sequence: Synthetic peptide
RNHDMG

SEQ ID NO: 92 - protein, 17 aa, synthetic construct
  REGION 1..17: note = Description of Artificial Sequence: Synthetic peptide
AISGSGGSTYYADSVKG

SEQ ID NO: 93 - protein, 11 aa, synthetic construct
  REGION 1..11: note = Description of Artificial Sequence: Synthetic peptide
EPKPMDTEFDY

SEQ ID NO: 94 - protein, 6 aa, synthetic construct
  REGION 1..6: note = Description of Artificial Sequence: Synthetic peptide
RNHDMG

SEQ ID NO: 95 - protein, 17 aa, synthetic construct
  REGION 1..17: note = Description of Artificial Sequence: Synthetic peptide
AISGSGGSTYYADSVKG

SEQ ID NO: 96 - protein, 11 aa, synthetic construct
  REGION 1..11: note = Description of Artificial Sequence: Synthetic peptide
EPKPMDTEFDY

SEQ ID NO: 97 - protein, 6 aa, synthetic construct
  REGION 1..6: note = Description of Artificial Sequence: Synthetic peptide
RNHDMG

SEQ ID NO: 98 - protein, 17 aa, synthetic construct
  REGION 1..17: note = Description of Artificial Sequence: Synthetic peptide
AISGSGGSTYYADSVKG

SEQ ID NO: 99 - protein, 11 aa, synthetic construct
  REGION 1..11: note = Description of Artificial Sequence: Synthetic peptide
EPKPMDTEFDY

SEQ ID NO: 100 - protein, 6 aa, synthetic construct
  REGION 1..6: note = Description of Artificial Sequence: Synthetic peptide
RNHDMG

SEQ ID NO: 101 - protein, 17 aa, synthetic construct
  REGION 1..17: note = Description of Artificial Sequence: Synthetic peptide
AISGSGGSTYYADSVKG

SEQ ID NO: 102 - protein, 11 aa, synthetic construct
  REGION 1..11: note = Description of Artificial Sequence: Synthetic peptide
EPKPMDTEFDY

SEQ ID NO: 103 - protein, 6 aa, synthetic construct
  REGION 1..6: note = Description of Artificial Sequence: Synthetic peptide
RNHDMG

SEQ ID NO: 104 - protein, 17 aa, synthetic construct
  REGION 1..17: note = Description of Artificial Sequence: Synthetic peptide
AISGSGGSTYYADSVKG

SEQ ID NO: 105 - protein, 11 aa, synthetic construct
  REGION 1..11: note = Description of Artificial Sequence: Synthetic peptide
EPKPMDTEFDY

SEQ ID NO: 106 - protein, 6 aa, synthetic construct
  REGION 1..6: note = Description of Artificial Sequence: Synthetic peptide
RNHDMG

SEQ ID NO: 107 - protein, 17 aa, synthetic construct
  REGION 1..17: note = Description of Artificial Sequence: Synthetic peptide
AISGSGGSTYYADSVKG

SEQ ID NO: 108 - protein, 11 aa, synthetic construct
  REGION 1..11: note = Description of Artificial Sequence: Synthetic peptide
EPKPMDTEFDY

SEQ ID NO: 109 - protein, 6 aa, synthetic construct
  REGION 1..6: note = Description of Artificial Sequence: Synthetic peptide
RNHDMG

SEQ ID NO: 110 - protein, 17 aa, synthetic construct
  REGION 1..17: note = Description of Artificial Sequence: Synthetic peptide
AISGSGGGTYYADSVKG

SEQ ID NO: 111 - protein, 11 aa, synthetic construct
  REGION 1..11: note = Description of Artificial Sequence: Synthetic peptide
EPSHFDRPFDY

SEQ ID NO: 112 - protein, 6 aa, synthetic construct
  REGION 1..6: note = Description of Artificial Sequence: Synthetic peptide
RNHDMG

SEQ ID NO: 113 - protein, 17 aa, synthetic construct
  REGION 1..17: note = Description of Artificial Sequence: Synthetic peptide
AISGSGGSTYYANSVKG

SEQ ID NO: 114 - protein, 11 aa, synthetic construct
  REGION 1..11: note = Description of Artificial Sequence: Synthetic peptide
EPSHFDRPFDY

SEQ ID NO: 115 - protein, 6 aa, synthetic construct
  REGION 1..6: note = Description of Artificial Sequence: Synthetic peptide
PMKDMG

SEQ ID NO: 116 - protein, 17 aa, synthetic construct
  REGION 1..17: note = Description of Artificial Sequence: Synthetic peptide
AISGSGGGTYYADSVKG

SEQ ID NO: 117 - protein, 11 aa, synthetic construct
  REGION 1..11: note = Description of Artificial Sequence: Synthetic peptide
EPKPMDTEFDY

SEQ ID NO: 118 - protein, 6 aa, synthetic construct
  REGION 1..6: note = Description of Artificial Sequence: Synthetic peptide
PMKDMG

SEQ ID NO: 119 - protein, 17 aa, synthetic construct
  REGION 1..17: note = Description of Artificial Sequence: Synthetic peptide
AISGSGGGTYYADSVKG

SEQ ID NO: 120 - protein, 11 aa, synthetic construct
  REGION 1..11: note = Description of Artificial Sequence: Synthetic peptide
EPKPMDTEFDY

SEQ ID NO: 121 - protein, 6 aa, synthetic construct
  REGION 1..6: note = Description of Artificial Sequence: Synthetic peptide
PMKDMG

SEQ ID NO: 122 - protein, 17 aa, synthetic construct
  REGION 1..17: note = Description of Artificial Sequence: Synthetic peptide
AISGSGGGTYYADSVKG

SEQ ID NO: 123 - protein, 11 aa, synthetic construct
  REGION 1..11: note = Description of Artificial Sequence: Synthetic peptide
EPKPMDTEFDY

SEQ ID NO: 124 - protein, 6 aa, synthetic construct
  REGION 1..6: note = Description of Artificial Sequence: Synthetic peptide
PMKDMG

SEQ ID NO: 125 - protein, 17 aa, synthetic construct
  REGION 1..17: note = Description of Artificial Sequence: Synthetic peptide
AISGSGGGTYYADSVKG

SEQ ID NO: 126 - protein, 11 aa, synthetic construct
  REGION 1..11: note = Description of Artificial Sequence: Synthetic peptide
EPKPMDTEFDY

SEQ ID NO: 127 - protein, 6 aa, synthetic construct
  REGION 1..6: note = Description of Artificial Sequence: Synthetic peptide
DNVEMS

SEQ ID NO: 128 - protein, 17 aa, synthetic construct
  REGION 1..17: note = Description of Artificial Sequence: Synthetic peptide
SIGTKGEYTYYADSVKG

SEQ ID NO: 129 - protein, 12 aa, synthetic construct
  REGION 1..12: note = Description of Artificial Sequence: Synthetic peptide
QTVNVPEPAFAY

SEQ ID NO: 130 - protein, 6 aa, synthetic construct
  REGION 1..6: note = Description of Artificial Sequence: Synthetic peptide
DNVEMS

SEQ ID NO: 131 - protein, 17 aa, synthetic construct
  REGION 1..17: note = Description of Artificial Sequence: Synthetic peptide
SIGSKGEYTYYADSVKG

SEQ ID NO: 132 - protein, 12 aa, synthetic construct
  REGION 1..12: note = Description of Artificial Sequence: Synthetic peptide
QTVNVPEPAFAY

SEQ ID NO: 133 - protein, 6 aa, synthetic construct
  REGION 1..6: note = Description of Artificial Sequence: Synthetic peptide
PMKDMG

SEQ ID NO: 134 - protein, 17 aa, synthetic construct
  REGION 1..17: note = Description of Artificial Sequence: Synthetic peptide
AISGSGGGTYYADSVKG

SEQ ID NO: 135 - protein, 11 aa, synthetic construct
  REGION 1..11: note = Description of Artificial Sequence: Synthetic peptide
EPSHFDRPFDY

SEQ ID NO: 136 - protein, 6 aa, synthetic construct
  REGION 1..6: note = Description of Artificial Sequence: Synthetic peptide
PMKDMG

SEQ ID NO: 137 - protein, 17 aa, synthetic construct
  REGION 1..17: note = Description of Artificial Sequence: Synthetic peptide
AISGSGGGTYYANSVKG

SEQ ID NO: 138 - protein, 11 aa, synthetic construct
  REGION 1..11: note = Description of Artificial Sequence: Synthetic peptide
EPSHFDRPFDY

SEQ ID NO: 139 - protein, 29 aa, synthetic construct
  REGION 1..29: note = Description of Artificial Sequence: Synthetic peptide
EVQLLESGGGLVQPGGSLRLSCAASGFTF

SEQ ID NO: 140 - protein, 29 aa, synthetic construct
  REGION 1..29: note = Description of Artificial Sequence: Synthetic peptide
EVQLLESGGGLVQPGGSLRLSCAASGFRI

SEQ ID NO: 141 - protein, 29 aa, synthetic construct
  REGION 1..29: note = Description of Artificial Sequence: Synthetic peptide
EVQLLESGGGLVQPGGSLRLSCAASGFTL

SEQ ID NO: 142 - protein, 29 aa, synthetic construct
  REGION 1..29: note = Description of Artificial Sequence: Synthetic peptide
EVQLLESGGGLVQPGGSLRLSCAASGYTF

SEQ ID NO: 143 - protein, 29 aa, synthetic construct
  REGION 1..29: note = Description of Artificial Sequence: Synthetic peptide
EVQLLESGGGLVQPGGSLRLTCAASGFSF

SEQ ID NO: 144 - protein, 29 aa, synthetic construct
  REGION 1..29: note = Description of Artificial Sequence: Synthetic peptide
EVQMLESGGGLVQPGESLRLSCAASGFTF

SEQ ID NO: 145 - protein, 14 aa, synthetic construct
  REGION 1..14: note = Description of Artificial Sequence: Synthetic peptide
WVRQAPGKGLEWVS

SEQ ID NO: 146 - protein, 14 aa, synthetic construct
  REGION 1..14: note = Description of Artificial Sequence: Synthetic peptide
WVRQAPGKGLEWAS

SEQ ID NO: 147 - protein, 14 aa, synthetic construct
  REGION 1..14: note = Description of Artificial Sequence: Synthetic peptide
WARQAPGKGLEWVS

SEQ ID NO: 148 - protein, 31 aa, synthetic construct
  REGION 1..31: note = Description of Artificial Sequence: Synthetic polypeptide
RFTISRDNSKNTLYLQMNSLRAEDTAVYYCA

SEQ ID NO: 149 - protein, 31 aa, synthetic construct
  REGION 1..31: note = Description of Artificial Sequence: Synthetic polypeptide
RFTISRDNSRNTLYLQMNSLRAEDTAVYYCA

SEQ ID NO: 150 - protein, 31 aa, synthetic construct
  REGION 1..31: note = Description of Artificial Sequence: Synthetic polypeptide
RFTISRDNSKNTLYLQMNSMRAEDTAVYYCA

SEQ ID NO: 151 - protein, 31 aa, synthetic construct
  REGION 1..31: note = Description of Artificial Sequence: Synthetic polypeptide
RFTISRDNSKNTLYLQMNSPRAEDTAVYYCA

SEQ ID NO: 152 - protein, 31 aa, synthetic construct
  REGION 1..31: note = Description of Artificial Sequence: Synthetic polypeptide
RFTISRDDSKNTLYLQMNSLRAEDTAVYYCA

SEQ ID NO: 153 - protein, 11 aa, synthetic construct
  REGION 1..11: note = Description of Artificial Sequence: Synthetic peptide
WGQGTLVTVSS

SEQ ID NO: 154 - protein, 11 aa, synthetic construct
  REGION 1..11: note = Description of Artificial Sequence: Synthetic peptide
WGQGTLVTVLS

SEQ ID NO: 155 - protein, 11 aa, synthetic construct
  REGION 1..11: note = Description of Artificial Sequence: Synthetic peptide
RSPGTLVTVSS

SEQ ID NO: 156 - protein, 11 aa, synthetic construct
  REGION 1..11: note = Description of Artificial Sequence: Synthetic peptide
PSPGTQVTVSS

SEQ ID NO: 157 - protein, 11 aa, synthetic construct
  REGION 1..11: note = Description of Artificial Sequence: Synthetic peptide
PSPGTLVTVSS

SEQ ID NO: 158 - protein, 11 aa, synthetic construct
  REGION 1..11: note = Description of Artificial Sequence: Synthetic peptide
RSQGTLVTVSS

SEQ ID NO: 159 - protein, 11 aa, synthetic construct
  REGION 1..11: note = Description of Artificial Sequence: Synthetic peptide
WSQGTLVTVSS

SEQ ID NO: 160 - protein, 11 aa, synthetic construct
  REGION 1..11: note = Description of Artificial Sequence: Synthetic peptide
RGQGTLVTVSS

SEQ ID NO: 161 - protein, 11 aa, synthetic construct
  REGION 1..11: note = Description of Artificial Sequence: Synthetic peptide
RFQGTLVTVSS

SEQ ID NO: 162 - protein, 11 aa, synthetic construct
  REGION 1..11: note = Description of Artificial Sequence: Synthetic peptide
WSPGTLVTVSS

SEQ ID NO: 163 - protein, 11 aa, synthetic construct
  REGION 1..11: note = Description of Artificial Sequence: Synthetic peptide
GSPGTLVTVSS

SEQ ID NO: 164 - protein, 11 aa, synthetic construct
  REGION 1..11: note = Description of Artificial Sequence: Synthetic peptide
WGPGTLVTVSS

SEQ ID NO: 165 - protein, 11 aa, synthetic construct
  REGION 1..11: note = Description of Artificial Sequence: Synthetic peptide
RGPGTLVTVSS

SEQ ID NO: 166 - protein, 11 aa, synthetic construct
  REGION 1..11: note = Description of Artificial Sequence: Synthetic peptide
CGPGTLVTVSS

SEQ ID NO: 167 - protein, 11 aa, synthetic construct
  REGION 1..11: note = Description of Artificial Sequence: Synthetic peptide
RSCGTLVTVSS

SEQ ID NO: 168 - protein, 11 aa, synthetic construct
  REGION 1..11: note = Description of Artificial Sequence: Synthetic peptide
RSPGTLVTVLE

SEQ ID NO: 169 - protein, 11 aa, synthetic construct
  REGION 1..11: note = Description of Artificial Sequence: Synthetic peptide
PSPGTLVTVLE

SEQ ID NO: 170 - protein, 11 aa, synthetic construct
  REGION 1..11: note = Description of Artificial Sequence: Synthetic peptide
RSQGTLVTVSS

SEQ ID NO: 171 - protein, 119 aa, synthetic construct
  REGION 1..119: note = Description of Artificial Sequence: Synthetic polypeptide
EVQLLESGGGLVQPGGSLRLSCAASGFRIRNHDMGWVRQAPGKGLEWVSAISGSGGSTYYADSVKGRFTISRDNSKNTLYLQMNSLRAEDTAVYYCAEPKPMDTEFDYWGQGTLVTVSS

SEQ ID NO: 172 - protein, 120 aa, synthetic construct
  REGION 1..120: note = Description of Artificial Sequence: Synthetic polypeptide
EVQLLESGGGLVQPGGSLRLSCAASGFTFDNVEMSWVRQAPGKGLEWVSSIGSKGEDTYYADSVKGRFTISRDNSKNTLYLQMNSLRAEDTAVYYCAQTVNVPEPAFAYWGQGTLVTVSS

SEQ ID NO: 173 - protein, 119 aa, synthetic construct
  REGION 1..119: note = Description of Artificial Sequence: Synthetic polypeptide
EVQLLESGGGLVQPGGSLRLSCAASGFTFPMKDMGWVRQAPGKGLEWVSAISGSGGSTYYADSVKGRFTISRDNSKNTLYLQMNSLRAEDTAVYYCAEPSHFDRPFDYWGQGTLVTVSS

SEQ ID NO: 174 - protein, 119 aa, synthetic construct
  REGION 1..119: note = Description of Artificial Sequence: Synthetic polypeptide
EVQLLESGGGLVQPGGSLRLSCAASGFTFRNHDMGWVRQAPGKGLEWVSAISGSGGSTYYANSVKGRFTISRDNSKNTLYLQMNSLRAEDTAVYYCAEPKPMDTEFDYRSPGTLVTVSS

SEQ ID NO: 175 - protein, 119 aa, synthetic construct
  REGION 1..119: note = Description of Artificial Sequence: Synthetic polypeptide
EVQLLESGGGLVQPGGSLRLSCAASGFTFRNHDMGWVRQAPGKGLEWVSAISGSGGSTYYADSVKGRFTISRDNSKNTLYLQMNSLRAEDTAVYYCAEPKPMDTEFDYRSPGTLVTVSS

SEQ ID NO: 176 - protein, 119 aa, synthetic construct
  REGION 1..119: note = Description of Artificial Sequence: Synthetic polypeptide
EVQLLESGGGLVQPGGSLRLSCAASGFTFRNHDMGWVRQAPGKGLEWVSAISGSGGSTYYADSVKGRFTISRDNSKNTLYLQMNSLRAEDTAVYYCAEPKPMDTEFDYRSQGTLVTVSS

SEQ ID NO: 177 - protein, 119 aa, synthetic construct
  REGION 1..119: note = Description of Artificial Sequence: Synthetic polypeptide
EVQLLESGGGLVQPGGSLRLSCAASGFTFRNHDMGWVRQAPGKGLEWVSAISGSGGSTYYANSVKGRFTISRDNSKNTLYLQMNSLRAEDTAVYYCAEPKPMDTEFDYPSPGTQVTVSS

SEQ ID NO: 178 - protein, 119 aa, synthetic construct
  REGION 1..119: note = Description of Artificial Sequence: Synthetic polypeptide
EVQLLESGGGLVQPGGSLRLSCAASGFTFRNHDMGWVRQAPGKGLEWVSAISGSGGSTYYANSVKGRFTISRDNSKNTLYLQMNSLRAEDTAVYYCAEPKPMDTEFDYPSPGTLVTVSS

SEQ ID NO: 179 - protein, 119 aa, synthetic construct; identical to SEQ ID NO: 176
  REGION 1..119: note = Description of Artificial Sequence: Synthetic polypeptide

SEQ ID NO: 180 - protein, 119 aa, synthetic construct
  REGION 1..119: note = Description of Artificial Sequence: Synthetic polypeptide
EVQLLESGGGLVQPGGSLRLTCAASGFSFRNHDMGWVRQAPGKGLEWVSAISGSGGSTYYADSVKGRFTISRDNSKNTLYLQMNSLRAEDTAVYYCAEPKPMDTEFDYRSPGTLVTVSS

SEQ ID NO: 181 - protein, 119 aa, synthetic construct
  REGION 1..119: note = Description of Artificial Sequence: Synthetic polypeptide
EVQLLESGGGLVQPGGSLRLSCAASGFTFRNHDMGWVRQAPGKGLEWVSAISGSGGSTYYADSVKGRFTISRDDSKNTLYLQMNSLRAEDTAVYYCAEPKPMDTEFDYRSPGTLVTVSS

SEQ ID NO: 182 - protein, 119 aa, synthetic construct
  REGION 1..119: note = Description of Artificial Sequence: Synthetic polypeptide
EVQLLESGGGLVQPGGSLRLSCAASGFTFRNHDMGWVRQAPGKGLEWVSAISGSGGSTYYADSVKGRFTISRDNSKNTLYLQMNSLRAEDTAVYYCAEPKPMDTEFDYFSQGTLVTVSS

SEQ ID NO: 183 - protein, 119 aa, synthetic construct
  REGION 1..119: note = Description of Artificial Sequence: Synthetic polypeptide
EVQLLESGGGLVQPGGSLRLSCAASGFTFRNHDMGWVRQAPGKGLEWVSAISGSGGSTYYADSVKGRFTISRDNSKNTLYLQMNSLRAEDTAVYYCAEPKPMDTEFDYRFQGTLVTVSS

SEQ ID NO: 184 - protein, 119 aa, synthetic construct
  REGION 1..119: note = Description of Artificial Sequence: Synthetic polypeptide
EVQLLESGGGLVQPGGSLRLSCAASGFTFRNHDMGWVRQAPGKGLEWVSAISGSGGSTYYADSVKGRFTISRDNSKNTLYLQMNSLRAEDTAVYYCAEPKPMDTEFDYWSPGTLVTVSS

SEQ ID NO: 185 - protein, 119 aa, synthetic construct
  REGION 1..119: note = Description of Artificial Sequence: Synthetic polypeptide
EVQLLESGGGLVQPGGSLRLSCAASGFTFRNHDMGWVRQAPGKGLEWVSAISGSGGSTYYADSVKGRFTISRDNSKNTLYLQMNSLRAEDTAVYYCAEPKPMDTEFDYRFPGTLVTVSS

SEQ ID NO: 186 - protein, 119 aa, synthetic construct
  REGION 1..119: note = Description of Artificial Sequence: Synthetic polypeptide
EVQLLESGGGLVQPGGSLRLSCAASGFTFRNHDMGWVRQAPGKGLEWVSAISGSGGSTYYADSVKGRFTISRDNSKNTLYLQMNSLRAEDTAVYYCAEPKPMDTEFDYRGPGTLVTVSS

SEQ ID NO: 187 - protein, 119 aa, synthetic construct
  REGION 1..119: note = Description of Artificial Sequence: Synthetic polypeptide
EVQLLESGGGLVQPGGSLRLSCAASGFTFRNHDMGWVRQAPGKGLEWVSAISGSGGSTYYADSVKGRFTISRDNSKNTLYLQMNSLRAEDTAVYYCAEPKPMDTEFDYCGPGTLVTVSS

SEQ ID NO: 188 - protein, 119 aa, synthetic construct
  REGION 1..119: note = Description of Artificial Sequence: Synthetic polypeptide
EVQLLESGGGLVQPGGSLRLSCAASGYTFPMKDMGWVRQAPGKGLEWVSAISGSGGGTYYADSVKGRFTISRDNSKNTLYLQMNSLRAEDTAVYYCAEPKPMDTEFDYRSQGTLVTVSS

SEQ ID NO: 189 - protein, 119 aa, synthetic construct
  REGION 1..119: note = Description of Artificial Sequence: Synthetic polypeptide
EVQLLESGGGLVQPGGSLRLSCAASGYTFPMKDMGWVRQAPGKGLEWVSAISGSGGGTYYADSVKGRFTISRDNSKNTLYLQMNSLRAEDTAVYYCAEPKPMDTEFDYRSCGTLVTVSS

SEQ ID NO: 190 - protein, 119 aa, synthetic construct
  REGION 1..119: note = Description of Artificial Sequence: Synthetic polypeptide
EVQLLESGGGLVQPGGSLRLSCAASGYTFPMKDMGWVRQAPGKGLEWVSAISGSGGGTYYADSVKGRFTISRDNSKNTLYLQMNSLRAEDTAVYYCAEPKPMDTEFDYRSPGTLVTVSS

SEQ ID NO: 191 - protein, 119 aa, synthetic construct
  REGION 1..119: note = Description of Artificial Sequence: Synthetic polypeptide
EVQLLESGGGLVQPGGSLRLSCAASGYTFPMKDMGWVRQAPGKGLEWVSAISGSGGGTYYADSVKGRFTISRDNSKNTLYLQMNSLRAEDTAVYYCAEPKPMDTEFDYPSPGTLVTVSS

SEQ ID NO: 192 - protein, 119 aa, synthetic construct
  REGION 1..119: note = Description of Artificial Sequence: Synthetic polypeptide
EVQLLESGGGLVQPGGSLRLSCAASGFTFRNHDMGWVRQAPGKGLEWVSAISGSGGGTYYADSVKGRFTISRDNSKNTLYLQMNSLRAEDTAVYYCAEPSHFDRPFDYRSQGTLVTVSS

SEQ ID NO: 193 - protein, 119 aa, synthetic construct
  REGION 1..119: note = Description of Artificial Sequence: Synthetic polypeptide
EVQLLESGGGLVQPGGSLRLSCAASGFTFRNHDMGWVRQAPGKGLEWVSAISGSGGSTYYANSVKGRFTISRDNSKNTLYLQMNSLRAEDTAVYYCAEPSHFDRPFDYRSQGTLVTVSS

SEQ ID NO: 194 - protein, 120 aa, synthetic construct
  REGION 1..120: note = Description of Artificial Sequence: Synthetic polypeptide
EVQMLESGGGLVQPGGSLRLSCAASGFTFDNVEMSWVRQAPGKGLEWVSSIGTKGEYTYYADSVKGRFTISRDNSKNTLYLQMNSMRAEDTAVYYCAQTVNVPEPAFAYWGQGTLVTVLS

SEQ ID NO: 195 - protein, 120 aa, synthetic construct
  REGION 1..120: note = Description of Artificial Sequence: Synthetic polypeptide
EVQMLESGGGLVQPGESLRLSCAASGFTFDNVEMSWVRQAPGKGLEWVSSIGSKGEYTYYADSVKGRFTISRDNSKNTLYLQMNSPRAEDTAVYYCAQTVNVPEPAFAYWGQGTLVTVSS

SEQ ID NO: 196 - protein, 119 aa, synthetic construct
  REGION 1..119: note = Description of Artificial Sequence: Synthetic polypeptide
EVQLLESGGGLVQPGGSLRLSCAASGYTFPMKDMGWVRQAPGKGLEWVSAISGSGGGTYYADSVKGRFTISRDNSKNTLYLQMNSLRAEDTAVYYCAEPSHFDRPFDYRSQGTLVTVSS

SEQ ID NO: 197 - protein, 119 aa, synthetic construct
  REGION 1..119: note = Description of Artificial Sequence: Synthetic polypeptide
EVQLLESGGGLVQPGGSLRLSCAASGYTFPMKDMGWVRQAPGKGLEWVSAISGSGGGTYYANSVKGRFTISRDNSKNTLYLQMNSLRAEDTAVYYCAEPSHFDRPFDYRSQGTLVTVSS

SEQ ID NO: 198 - DNA, 6520 bp, synthetic construct
  misc_feature 1..6520: note = Description of Artificial Sequence: Synthetic polynucleotide
  source 1..6520: mol_type = other DNA
aattcgccgccaccatggagaccgacaccctgctgctgtgggtgctgctgctgtgggtgcccggatccaccggcgaggtgcagctgttggagtctgggggaggcttggtacagcctggggggtccctgcgtctctcctgtgcagcctccggattcacctttcgtaatcatgatatggggtgggtccgccaggctccagggaagggtctagagtgggtctcagctattagtggtagtggtggtagcacatactacgcaaactccgtgaagggccggttcaccatctcccgcgacaattccaagaacacgctgtatctgcaaatgaacagcctgcgtgccgaggacaccgcggtatattactgtgcggaaccgaagcctatggatacggagtttgactacaggagtccgggaaccctggtcaccgtctcgagcgctagcacccacacctgccccccctgccctgcccccgagctgctgggcggacctagcgtgttcctgttcccccccaagcctaaggacaccctgatgatcagcaggacccccgaagtgacctgcgtggtggtggatgtgagccacgaggaccctgaagtgaagttcaactggtacgtggacggcgtggaagtgcacaacgccaagaccaagcccagagaggagcagtacaacagcacctaccgcgtggtgtctgtgctgaccgtgctgcaccaggattggctgaacggcaaggagtacaagtgcaaagtgagcaacaaggccctgcctgcccctatcgagaaaaccatcagcaaggccaagggccagcctagagagccccaggtctacaccctgcctccctccagagatgagctgaccaagaaccaggtgtccctgacctgtctggtgaagggcttctaccccagcgacatcgccgtggagtgggagagcaacggccagcccgagaacaactacaagaccaccccccctgtgctggacagcgatggcagcttcttcctgtactccaagctgaccgtggacaagagcagatggcagcagggcaacgtgttcagctgcagcgtgatgcacgaggccctgcacaatcactacacccagaagagtctgagcctgtcccctggcaagtgatagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtgg

SEQ ID NO: 199 - skipped

SEQ ID NO: 200 - protein, 17 aa, synthetic construct
  REGION 1..17: note = Description of Artificial Sequence: Synthetic peptide
  VARIANT 8: note = X=Ser or Gly
  VARIANT 13: note = X=Asp or Asn
AISGSGGXTYYAXSVKG

SEQ ID NO: 201 - protein, 11 aa, synthetic construct
  REGION 1..11: note = Description of Artificial Sequence: Synthetic peptide
  VARIANT 3: note = X=Lys or Ser
  VARIANT 4: note = X=Pro or His
  VARIANT 5: note = X=Met or Phe
  VARIANT 7: note = X=Thr or Arg
  VARIANT 8: note = X=Glu or Pro
EPXXXDXXFDY

SEQ ID NO: 202 - protein, 11 aa, synthetic construct
  REGION 1..11: note = Description of Artificial Sequence: Synthetic peptide
  VARIANT 1: note = X=Trp, Arg, Pro, Gly or Cys
  VARIANT 2: note = X=Gly, Ser or Phe
  VARIANT 3: note = X=Gln, Pro or Cys
  VARIANT 6: note = X=Leu or Gln
  VARIANT 10: note = X=Ser or Leu
  VARIANT 11: note = X=Ser or Glu
XXXGTXVTVXX

SEQ ID NO: 203 - protein, 29 aa, synthetic construct
  REGION 1..29: note = Description of Artificial Sequence: Synthetic peptide
  VARIANT 7: note = X=Ser or Pro
  VARIANT 27: note = X=Tyr, Phe or Val
  VARIANT 28: note = X=Arg, Thr or Asn
  VARIANT 29: note = X=Ile, Phe or Val
EVQLLEXGGGLVQPGGSLRLSCAASGXXX

SEQ ID NO: 204 - protein, 14 aa, synthetic construct
  REGION 1..14: note = Description of Artificial Sequence: Synthetic peptide
  VARIANT 2: note = X=Val or Ala
  VARIANT 13: note = X=Val or Ala
WXRQAPGKGLEWXS

SEQ ID NO: 205 - protein, 31 aa, synthetic construct
  REGION 1..31: note = Description of Artificial Sequence: Synthetic polypeptide
  VARIANT 10: note = X=Arg or Lys
  VARIANT 20: note = X=Leu or Met
RFTISRDNSXNTLYLQMNSXRAEDTAVYYCA

SEQ ID NO: 206 - protein, 17 aa, synthetic construct
  REGION 1..17: note = Description of Artificial Sequence: Synthetic peptide
  VARIANT 1: note = X=Ala or Ser
  VARIANT 3: note = X=Ser or Gly
  VARIANT 4: note = X=Gly, Ser or Thr
  VARIANT 5: note = X=Ser or Lys
  VARIANT 7: note = X=Gly or Glu
  VARIANT 8: note = X=Ser, Gly, Asp or Tyr
  VARIANT 13: note = X=Asp or Asn
XIXXXGXXTYYAXSVKG

SEQ ID NO: 207 - protein, 31 aa, synthetic construct
  REGION 1..31: note = Description of Artificial Sequence: Synthetic polypeptide
  VARIANT 20: note = X=Leu or Met
RFTISRDNSKNTLYLQMNSXRAEDTAVYYCA

SEQ ID NO: 208 - protein, 6 aa, synthetic construct
  REGION 1..6: note = Description of Artificial Sequence: Synthetic 6xHis tag
HHHHHH

SEQ ID NO: 209 - protein, 6 aa, Homo sapiens
SSYAMS

SEQ ID NO: 210 - protein, 17 aa, Homo sapiens
AISGSGGSTYYADSVKG

SEQ ID NO: 211 - protein, 10 aa, Homo sapiens
CAKSYGAFDY

SEQ ID NO: 212 - protein, 13 aa, synthetic construct
  REGION 1..13: note = Description of Artificial Sequence: Synthetic peptide
CAEPKPMDTEFDY

SEQ ID NO: 213 - protein, 13 aa, synthetic construct
  REGION 1..13: note = Description of Artificial Sequence: Synthetic peptide
CAEPSHFDRPFDY

SEQ ID NO: 214 - protein, 14 aa, synthetic construct
  REGION 1..14: note = Description of Artificial Sequence: Synthetic peptide
CAQTVNVPEPAFAY

SEQ ID NO: 215 - protein, 16 aa, synthetic construct
  REGION 1..16: note = Description of Artificial Sequence: Synthetic peptide
CAKQRGLNRYRAQFDY

SEQ ID NO: 216 - protein, 15 aa, synthetic construct
  REGION 1..15: note = Description of Artificial Sequence: Synthetic peptide
CAKQGQISNFPRFDY

SEQ ID NO: 217 - protein, 16 aa, synthetic construct
  REGION 1..16: note = Description of Artificial Sequence: Synthetic peptide
CAKFNRFSHRQYNFDY

SEQ ID NO: 218 - protein, 16 aa, synthetic construct
  REGION 1..16: note = Description of Artificial Sequence: Synthetic peptide
CAKVHTFANRSLNFDY

SEQ ID NO: 219 - protein, 6 aa, synthetic construct
  REGION 1..6: note = Description of Artificial Sequence: Synthetic peptide
GAYSMS

SEQ ID NO: 220 - protein, 16 aa, synthetic construct
  REGION 1..16: note = Description of Artificial Sequence: Synthetic peptide
CAKCSSCTSLNANFDY

SEQ ID NO: 221 - protein, 14 aa, synthetic construct
  REGION 1..14: note = Description of Artificial Sequence: Synthetic peptide
CAKASAPKYFRFDY

SEQ ID NO: 222 - protein, 15 aa, synthetic construct
  REGION 1..15: note = Description of Artificial Sequence: Synthetic peptide
CAKPWRVYSYDRFDY

SEQ ID NO: 223 - protein, 16 aa, synthetic construct
  REGION 1..16: note = Description of Artificial Sequence: Synthetic peptide
CAKLQRYDRYTLNFDY

SEQ ID NO: 224 - protein, 127 aa, synthetic construct
  REGION 1..127: note = Description of Artificial Sequence: Synthetic polypeptide
EVQLLEPGGGLVQPGGSLRLSCAASGVNVSHDSMTWVRQAPGKGLEWVSAIRGPNGSTYYADSVKGRFTISRDNSKNTLYLQMNSLRAEDTAVYYCASGARHADTERPPSQQTMPFWGQGTLVTVSS